(19) United States
(12) Patent Application Publication
(10) Pub. No.: US 2016/0000499 A1
(43) Pub. Date: Jan. 7, 2016

(54) ENDOVASCULAR CATHETERS FOR CAROTID BODY ABLATION UTILIZING AN IONIC LIQUID STREAM

(71) Applicant: CIBIEM, INC., Hayward, CA (US)

(72) Inventors: Charles LENNOX, Hudson, NH (US); Vahid SAADAT, Atherton, CA (US); Mark GELFAND, New York, NY (US)

(21) Appl. No.: 14/769,515

(22) PCT Filed: Mar. 12, 2014
(86) PCT No.: PCT/US2014/024462

§ 371 Date: Aug. 21, 2015

Related U.S. Application Data
(60) Provisional application No. 61/793,267, filed on Mar. 15, 2013.

(51) Int. Cl. A61B 18/14; A61N 7/00

(52) U.S. Cl. CPC A61B 18/1492; A61B 18/1445; A61N 7/00; A61B 2018/00077

(57) ABSTRACT

Methods and endovascular catheters for assessing, and treating patients having sympathetically mediated disease, involving augmented peripheral chemoreflex and heightened sympathetic tone by reducing chemosensor input to the nervous system via transmural carotid body modulation using a catheter with an ionic liquid stream electrode.

### CROSS REFERENCE TO RELATED APPLICATIONS

This application claims the priority of U.S. App. No. 61/793,267, filed Mar. 15, 2013, the disclosure of which is incorporated by reference herein.

This application is related to and incorporates by reference herein the disclosures of the following applications: U.S. application Ser. No. 14/188,452, filed Feb. 24, 2014; and 61/924,067, filed Jan. 6, 2014.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present disclosure is directed generally to devices, systems and methods for treating patients having sympathetically mediated disease associated at least in part with augmented peripheral chemoreflex, heightened sympathetic activation, or autonomic imbalance by ablating at least one peripheral chemoreceptor (e.g. carotid body) with an endovascular transmural ablation catheter comprising at least one virtual electrode.

### BACKGROUND

It is known that an imbalance of the autonomic nervous system is associated with several disease states. Restoration of autonomic balance has been a target of several medical treatments including modalities such as pharmacological, device-based, and electrical stimulation. For example, beta blockers are a class of drugs used to reduce sympathetic activity to treat cardiac arrhythmias and hypertension; Gelfand and Levin (U.S. Pat. No. 7,162,303) describe a device-based treatment used to decrease renal sympathetic activity to treat heart failure, hypertension, and renal failure; Yun and Yuarn-Bor (U.S. Pat. No. 7,149,574; U.S. Pat. No. 7,363,076; U.S. Pat. No. 7,738,952) describe a method of restoring autonomic balance by increasing parasympathetic activity to treat disease associated with parasympathetic attrition; Kieval, Burns and Serdar (U.S. Pat. No. 8,060,206) describe an electrical pulse generator that stimulates a baroreceptor, increasing parasympathetic activity, in response to high blood pressure; Hlavka and Elliott (US 2010/0070004) describe an implantable electrical stimulator in communication with an afferent neural pathway of a carotid body chemoreceptor to control dyspnea via electrical neuromodulation. More recently, Carotid Body Ablation (CBA) has been conceived for treating sympathetically mediated diseases.

Ablating the carotid body in a human patient is risky and difficult. The carotid body is typically about the size of a grain of rice, located near other glands, nerves, muscles and other organs, and moves with movement of the jaw and neck, respiration and blood pulsation. Conventional open surgical techniques to access the carotid body directly through the neck are challenging due to the nerves, muscles, arteries, veins and other organs near the carotid body.

### SUMMARY

There is a desire for minimally invasive surgical techniques and instruments to ablate the carotid body. Endovascular catheter assemblies are known for performing minimally invasive surgeries on the heart, kidney and other body organs typically located below the neck. These catheter assemblies tend to be too short, too large and otherwise not suited to reaching the neck and, particularly, the narrow blood vessels in the neck. Endovascular catheter assemblies are also known for treating arteries in the neck such as to treat aneurysms in the wall of a blood vessel.

It is not conventional to use minimally invasive surgical instruments and techniques to treat organs in the neck. A difficulty with applying minimally invasive surgical techniques to an organ in the neck, other than an artery or vein, is the long and tortuous path through the vascular system that a catheter must advance to reach the neck. Another difficulty is properly positioning the tip (distal end) of the catheter in an artery to act on the target organ, which is external to the artery. The organ may move with respect to the artery, the narrow arteries in the neck and the complex geometries of these arteries present challenges to a minimally invasive technique to reach the carotid body.

While catheter probes with stimulation electrodes have been proposed for electrically stimulating the carotid body (US Patent Application Publication 2012/0059437), ablating or otherwise permanently changing the carotid body is new. Ablating or otherwise permanently changing the carotid body or its function requires the application of energy, chemicals or other forces sufficient to damage the carotid body or its associated nerves and potentially tissue and blood vessel walls near the carotid body. Damaging the carotid body and nearby tissue is not necessary or desired if the object is to electrically stimulate the carotid body. Applying a relatively low level of energy to electrically stimulate the carotid body will unlikely damage a blood vessel or surrounding tissue, even if the energy is applied to a broader area than the carotid body. The level of energy and force or the chemicals needed to ablate the carotid body is substantially higher than the levels needed for stimulation. Applying energy, chemicals and forces sufficient to damage the carotid body raises concerns that the damage could extend to nearby nerves and other organs, rupture the wall of the blood vessel or create blood clots that could flow to the brain.

In view of the need to damage the carotid body, the requirements for positioning the tip of an ablating catheter in a carotid artery and narrowly target delivery of the energy, chemicals or force to the carotid body are strict. Recognizing and identifying the requirements for positioning an ablating tip of a catheter was a first step in inventing an endovascular catheter assembly for ablating the carotid body. A second step was to invent endovascular catheter assemblies that satisfied the requirements.

Some patients suffering from a sympathetically mediated disease who may benefit from a carotid body ablation procedure may have a significant amount of atheromatous plaque in their carotid arteries. Performing an endovascular procedure in the presence of plaque may pose a risk of brain embolism, particularly if the plaque is in the internal carotid artery, which feeds the brain, and the endovascular procedure involved significant mechanical manipulation in the internal carotid artery. Therefore, there may be a reduced risk benefit of an endovascular catheter configured to ablate a carotid body while minimizing mechanical manipulation or contact forces on a carotid artery wall or in association with plaque. Endovascular catheters have been conceived comprising a virtual electrode, that is, an electrode that delivers ablative energy via an ionic liquid stream, which may reduce mechanical manipulation or contact forces on a carotid artery wall or in association with plaque.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a hollow metallic cylindrical structure disposed in the vicinity of the distal end of the catheter shaft with at least one lateral fenestration and an electrically isolative coating disposed on the external surface, at least one lumen within the catheter shaft in communication with the interior of the hollow metallic cylindrical structure and a fluid connector disposed in the vicinity of the proximal end of the catheter shaft, at least a portion of the internal wall of the hollow metallic cylindrical structure configured as an electrode, and with the hollow metallic cylindrical structure connected to an electrical connector disposed in the vicinity of the proximal end of the catheter shaft by an electrical conduit.

A vascular catheter for ablation of perivascular tissue has been conceived comprising a catheter shaft configured for vascular use, a hollow non-metallic cylindrical structure disposed in the vicinity of the distal end of the catheter shaft with at least one lateral fenestration and an electrically conductive material disposed on the internal surface configured as an electrode, at least one lumen within the catheter shaft in communication with the interior of the hollow metallic cylindrical structure and a fluid connector disposed in the vicinity of the proximal end of the catheter shaft, and with the electrode connected to an electrical connector disposed in the vicinity of the proximal end of the catheter shaft by an electrical conduit.

A vascular catheter for ablation of perivascular tissue has been conceived comprising a catheter shaft configured for vascular use, a hollow cylindrical structure disposed in the vicinity of the distal end of the catheter shaft with at least one lateral fenestration, a mechanism configured for pressing the at least one lateral fenestration against a vascular wall disposed in the vicinity of the hollow cylindrical structure, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, a mechanism configured for pressing the at least one lateral fenestration against a vascular wall disposed in the vicinity of the hollow cylindrical structure comprising a deployable and retractable wire loop, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, a mechanism configured for pressing the at least one lateral fenestration against a vascular wall disposed in the vicinity of the hollow cylindrical structure comprising an inflatable structure, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, a mechanism configured for pressing the at least one lateral fenestration against a vascular wall disposed in the vicinity of the hollow cylindrical structure comprising a structure configured for radial expansion in response to axial compressive force, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, a mechanism configured for pressing the at least one lateral fenestration against a vascular wall disposed in the vicinity of the hollow cylindrical structure comprising a pull wire in communication between the hollow cylindrical structure, and an actuator disposed in the vicinity of the proximal end, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, a mechanism configured for pressing the at least one lateral fenestration against a vascular wall disposed in the vicinity of the hollow cylindrical structure which provides a user with a substantially unambiguous fluoroscopic indication of the position of the at least one lateral fenestration within a vascular structure, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one temperature sensor disposed in the vicinity of the at least one lateral fenestration configured for measuring a vascular wall temperature, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, a lumen distal to the hollow cylindrical structure configured for use with a guidewire where the proximal terminal of the guidewire lumen is distal to the hollow cylindrical structure, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, a lumen distal to the hollow cylindrical structure configured for use with a guidewire where the proximal terminal of the guidewire lumen is proximal to the hollow cylindrical structure, and where all external surfaces of the catheter assembly are electrically isolated from the at least one electrode surface.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, an elastomeric membrane comprising a slit covering the at least one fenestration configured for one-way fluid flow from within the hollow cylindrical structure, and where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, at least one lumen within the catheter shaft in communication with the interior of the inflatable structure and a fluid connector disposed in the vicinity of the proximal end of the catheter shaft, at least one electrode surface within the interior of the inflatable structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, a mechanism configured for pressing the at least one lateral fenestration against a vascular wall disposed in the vicinity of the inflatable structure, at least one lumen within the catheter shaft in communication with the interior of the inflatable structure and a fluid connector disposed in the vicinity of the proximal end of the catheter shaft, at least one electrode surface within the interior of the inflatable structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, a mechanism configured for pressing the at least one lateral fenestration against a vascular wall disposed in the vicinity of the inflatable structure, at least one lumen within the catheter shaft in communication with the interior of the inflatable structure and a fluid connector disposed in the vicinity of the proximal end of the catheter shaft, at least one electrode surface within the interior of the inflatable structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, a mechanism configured for pressing the at least one lateral fenestration against a vascular wall disposed in the vicinity of the inflatable structure comprising a second inflatable structure, at least one lumen within the catheter shaft in communication with the interior of the first inflatable structure and a fluid connector disposed in the vicinity of the proximal end of the catheter shaft, at least one lumen within the catheter shaft in communication with the interior of the second inflatable structure and a fluid connector disposed in the vicinity of the proximal end of the catheter shaft, at least one electrode surface within the interior of the first inflatable structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, a mechanism configured for pressing the at least one lateral fenestration against a vascular wall disposed in the vicinity of the inflatable structure comprising a structure configured for radial expansion in response to axial compressive force, at least one lumen within the catheter shaft in communication with the interior of the inflatable structure and a fluid connector disposed in the vicinity of the proximal end of the catheter shaft, at least one electrode surface within the interior of the inflatable structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, a mechanism configured for pressing the at least one lateral fenestration against a vascular wall disposed in the vicinity of the inflatable structure comprising a pull wire in communication between the inflatable structure, and an actuator disposed in the vicinity of the proximal end, at least one lumen within the catheter shaft in communication with the interior of the inflatable structure and a fluid connector disposed in the vicinity of the proximal end of the catheter shaft, at least one electrode surface within the interior of the inflatable structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, a mechanism configured for pressing the at least one lateral fenestration against a vascular wall disposed in the vicinity of the inflatable structure which provides a user with a substantially unambiguous fluoroscopic indication of the position of the at least one lateral fenestration within a vascular structure, at least one lumen within the catheter shaft in communication with the interior of the inflatable structure and a fluid connector disposed in the vicinity of the proximal end of the catheter shaft, at least one electrode surface within the interior of the inflatable structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, at least one lumen within the catheter shaft in communication with the interior of the inflatable structure and a fluid connector disposed in the vicinity of the proximal end of the catheter shaft, at least one electrode surface within the interior of the inflatable structure connected to an electrical connector disposed in the vicinity of the proximal end of the catheter shaft by an electrical conduit, a lumen distal to the inflatable structure configured for use with a guidewire, where the proximal terminal of the guidewire lumen is distal to the inflatable structure, and where the where all external surfaces of the catheter assembly are electrically isolated from the at least one electrode surface.

A vascular catheter for ablation of perivascular tissue has been conceived comprising a catheter shaft configured for vascular use, an inflatable structure disposed in the vicinity of the distal end of the catheter shaft with at least one lateral fenestration, at least one lumen within the catheter shaft in communication with the interior of the inflatable structure and a fluid connector disposed in the vicinity of the proximal end of the catheter shaft, at least one electrode surface within the interior of the inflatable structure connected to an electrical connector disposed in the vicinity of the proximal end of the catheter shaft by an electrical conduit, a lumen distal to the inflatable structure configured for use with a guidewire where the proximal terminal of the guidewire lumen is proximal to the inflatable structure, and where all external surfaces of the catheter assembly are electrically isolated from the at least one electrode surface.

A vascular catheter for ablation of perivascular tissue has been conceived comprising a catheter shaft configured for vascular use, a forceps structure disposed in the vicinity of the distal end of the catheter shaft with one forceps element comprising a hollow cylindrical structure with at least one lateral fenestration oriented in the direction of the opposing forceps element, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, and where all external surfaces of the catheter assembly are electrically isolated from the at least one electrode surface.

A vascular catheter for ablation of perivascular tissue has been conceived comprising a catheter shaft configured for vascular use, a forceps structure disposed in the vicinity of the distal end of the catheter shaft where both forceps elements comprise a hollow cylindrical structure with at least one lateral fenestration oriented in the direction of the opposing forceps element, at least one lumen within the catheter shaft in communication with the interior of each hollow cylindrical structure and a fluid connector disposed in the vicinity of the proximal end of the catheter shaft, at least one electrode surface within the interior of each hollow cylindrical structures connected to an electrical connector disposed in the vicinity of the proximal end of the catheter shaft by an electrical conduit where each conduit is configured for connection to opposing electrical poles of an RF generator, and where all external surfaces of the catheter assembly are electrically isolated from each electrode surface.

A vascular catheter for ablation of perivascular tissue has been conceived comprising a catheter shaft configured for vascular use, a forceps structure disposed in the vicinity of the distal end of the catheter shaft where both forceps elements comprise an inflatable structure with at least one lateral fenestration oriented in the direction of the opposing forceps element, at least one lumen within the catheter shaft in communication with the interior of each inflatable structure and a fluid connector disposed in the vicinity of the proximal end of the catheter shaft, at least one electrode surface within the interior of each inflatable structure connected to an electrical connector disposed in the vicinity of the proximal end of the catheter shaft by an electrical conduit where each conduit is configured for connection to opposing electrical poles of an RF generator, and where all external surfaces of the catheter assembly are electrically isolated from each electrode surface.

A vascular catheter for ablation of perivascular tissue has been conceived comprising a catheter shaft configured for vascular use, a forceps structure disposed in the vicinity of the distal end of the catheter shaft where at least one of the forceps elements comprise a porous structure of non-electrically conductive material, at least one lumen within the catheter shaft in communication with the porous structure and a fluid connector disposed in the vicinity of the proximal end of the catheter shaft, at least one electrode surface in contact with the porous structure connected to an electrical connector disposed in the vicinity of the proximal end of the catheter by an electrical conduit, and where all external surfaces of the catheter assembly are electrically isolated from the electrode surface.

A vascular catheter for ablation of perivascular tissue has been conceived comprising a catheter shaft configured for vascular use, a forceps structure disposed in the vicinity of the distal end of the catheter shaft where both forceps elements comprise a porous structure, at least one lumen within the catheter shaft in communication with each porous structure and a fluid connector disposed in the vicinity of the proximal end of the catheter shaft, at least one electrode surface in contact with each porous structures connected to an electrical connector disposed in the vicinity of the proximal end of the catheter shaft by an electrical conduit where each conduit is configured for connection to opposing electrical poles of an RF generator, and where all external surfaces of the catheter assembly are electrically isolated from each electrode surface.

A vascular catheter for ablation of perivascular tissue has been conceived comprising a catheter shaft configured for vascular use, a forceps structure disposed in the vicinity of the distal end of the catheter shaft with at least one of the forceps elements comprising a hollow cylindrical structure with at least one lateral fenestration oriented in the direction of the opposing forceps element, at least one fluid channel in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, an opposing forceps element comprising a forceps arm with an inflatable structure disposed in the vicinity of its distal end and a fluid channel in communication with the interior of the inflatable structure and a fluid connector disposed in the vicinity of the proximal end of the catheter shaft, and where all external surfaces of the catheter assembly are electrically isolated from the at least one electrode surface.

A vascular catheter for ablation of perivascular tissue has been conceived comprising a catheter shaft configured for vascular use, a forceps structure disposed in the vicinity of the distal end of the catheter shaft with at least one of the forceps elements comprising a hollow cylindrical structure with at least one lateral fenestration oriented in the direction of the opposing forceps element, at least one fluid channel in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, an opposing forceps element comprising a forceps arm with an inflatable structure with at least one fenestration oriented towards the opposing forceps element disposed in the vicinity of its distal end, a fluid channel in communication with the interior of the inflatable structure and a fluid connector disposed in the vicinity of the proximal end of the catheter shaft, an electrode disposed within the interior of the inflatable structure connected to an electrical connector disposed in the vicinity of the proximal end of the catheter shaft by an electrical conduit, where the electrical conduit connected to the electrode within the hollow cylindrical structure is configured for connection to the opposite pole of an RF generator than the electrical conduit connected to the electrode within the inflatable structure, and where all external surfaces of the catheter assembly are electrically isolated from either electrode surface.

A vascular catheter for ablation of perivascular tissue has been conceived comprising a catheter shaft configured for vascular use, a forceps structure disposed in the vicinity of the distal end of the catheter shaft with at least one of the forceps elements comprising a hollow cylindrical structure with at least one lateral fenestration oriented in the direction of the opposing forceps element, at least one fluid channel in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, an opposing forceps element comprising a forceps arm with a porous structure disposed in the vicinity of its distal end, a fluid channel in communication with the porous structure and a fluid connector disposed in the vicinity of the proximal end of the catheter shaft, an electrode in contact with the porous structure connected to an electrical connector disposed in the vicinity of the proximal end of the catheter shaft by an electrical conduit, where the electrical conduit connected to the electrode within the hollow cylindrical structure is configured for connection to the opposite pole of an RF generator than the electrical conduit connected to the electrode in contact with the porous structure, and where all external surfaces of the catheter assembly are electrically isolated from either electrode surface.

A vascular catheter for ablation of perivascular tissue has been conceived comprising a catheter shaft configured for vascular use, a forceps structure disposed in the vicinity of the distal end of the catheter shaft with one forceps element comprising a hollow cylindrical structure with at least one lateral fenestration oriented in the direction of the opposing forceps element, at least one lumen within the catheter shaft in communication with the interior of the hollow cylindrical structure and a fluid connector disposed in the vicinity of the proximal end of the catheter shaft, at least one electrode surface within the interior of the hollow cylindrical structure connected to an electrical connector disposed in the vicinity of the proximal end of the catheter shaft by an electrical conduit, where all external surfaces of the catheter assembly are electrically isolated from the at least one electrode surface, and where the opposing forceps element is configured for deployment from, and retraction into the hollow cylindrical structure.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one lumen within the catheter shaft in communication with the interior of the hollow cylindrical structure and a fluid connector disposed in the vicinity of the proximal end of the catheter shaft, at least one Piezo-electric element disposed within the interior of the hollow cylindrical structure connected to an electrical connector disposed in the vicinity of the proximal end of the catheter shaft by a coaxial electrical conduit, and where the Piezo-electric element configured for ultrasonic ablation of perivascular tissue adjacent to the fenestration or for sensing stimulated ultrasonic harmonic emissions from adjacent perivascular tissues.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one lumen within the catheter shaft in communication with the interior of the hollow cylindrical structure and a fluid connector disposed in the vicinity of the proximal end of the catheter shaft, at least one Piezo-electric element disposed within the interior of the hollow cylindrical structure connected to an electrical connector disposed in the vicinity of the proximal end of the catheter shaft by a coaxial electrical conduit, a mechanism for pressing the at least one lateral fenestration against a vascular wall that provides the user with a substantially unambiguous fluoroscopic indication of the position of the at least one lateral fenestration within a vascular structure, and where the Piezo-electric element configured for ultrasonic ablation of perivascular tissue adjacent to the fenestration or for sensing stimulated ultrasonic harmonic emissions from adjacent perivascular tissues.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one lumen within the catheter shaft in communication with the interior of the hollow cylindrical structure and a fluid connector disposed in the vicinity of the proximal end of the catheter shaft, an electrode surface disposed within the hollow cylindrical structure connected to an electrical connector disposed in the vicinity of the proximal end of the catheter shaft by an electrical conduit, at least one Piezoelectric element disposed within the interior of the hollow cylindrical structure connected to an electrical connector disposed in the vicinity of the proximal end of the catheter shaft by a coaxial electrical conduit, where the Piezo-electric element configured for ultrasonic ablation of perivascular tissue adjacent to the fenestration or for sensing stimulated ultrasonic harmonic emissions from adjacent perivascular tissues, the electrode is configured for RF ablation of perivascular tissue, and where all external surfaces of the catheter assembly are electrically isolated from the electrode and the Piezo-electric element.

A vascular catheter for ablation of perivascular tissue has been conceived comprising a catheter shaft configured for vascular use, a hollow cylindrical structure disposed in the vicinity of the distal end of the catheter shaft with at least one lateral fenestration, at least one lumen within the catheter shaft in communication with the interior of the hollow cylindrical structure and a fluid connector disposed in the vicinity of the proximal end of the catheter shaft, an electrode surface disposed within the hollow cylindrical structure connected to an electrical connector disposed in the vicinity of the proximal end of the catheter shaft by an electrical conduit, at least one Piezo-electric element disposed within the interior of the hollow cylindrical structure connected to an electrical connector disposed in the vicinity of the proximal end of the catheter shaft by a coaxial electrical conduit, where the Piezo-electric element configured for ultrasonic ablation of perivascular tissue adjacent to the fenestration or for sensing stimulated ultrasonic harmonic emissions from adjacent perivascular tissues, the electrode is configured for RF ablation of perivascular tissue, a mechanism for pressing the at least one lateral fenestration against a vascular wall that provides the user with a substantially unambiguous fluoroscopic indication of the position of the at least one lateral fenestration within a vascular structure, and where all external surfaces of the catheter assembly are electrically isolated from the electrode and the Piezo-electric element.

An apparatus for ablation of perivascular tissue has been conceived comprising a sheath configured to house and deploy in a distal direction from a position within a common carotid artery a first catheter into the associated external carotid artery, and a second catheter into the associated internal carotid artery, whereby said first catheter comprises a radiofrequency electrode connected to one pole of an RF generator, and a mechanical biasing means configured to press the electrode against the medial aspect of the external carotid artery proximate to a target carotid body, and said second catheter comprises a perforated, non-conductive balloon housing a radiofrequency electrode connected to the second pole of said RF generator, with the interior of said balloon connected to a pressurized source of ionic liquid, wherein, said perforated balloon in conjunction with said pressurized source of ionic liquid is configured to elude a stream of ionic liquid into the internal carotid artery during bipolar radiofrequency carotid body ablation.

An apparatus for ablation of perivascular tissue has been conceived comprising a sheath configured to house and deploy in a distal direction from a position within a common carotid artery a catheter into the associated external carotid artery, and a bladder against the medial aspect of the proximal internal carotid artery, whereby said catheter comprises a radiofrequency electrode connected to one pole of an RF generator, and a mechanical biasing means configured to press the electrode against the medial aspect of the external carotid artery proximate to a target carotid body, and said bladder comprises a perforated, non-conductive membrane housing a radiofrequency electrode connected to the second pole of said RF generator, with the interior of said bladder connected to a pressurized source of ionic liquid, wherein, said perforated bladder in conjunction with said pressurized source of ionic liquid is configured to elude a stream of ionic liquid into the internal carotid artery during bipolar radiofrequency carotid body ablation.

A vascular catheter for ablation of perivascular tissue has been conceived comprising a radiofrequency electrode connected to one pole of an RF generator, and a mechanical biasing means configured to press the electrode against the medial aspect of the external carotid artery proximate to a target carotid body, and a metallic perforated fluid port located proximal to said electrode, and radially aligned with said mechanical biasing means, and connected to the second pole of said RF generator, and a pressurized source of ionic liquid, wherein, said perforated metallic fluid port, in conjunction with said pressurized source of ionic liquid is configured to elude a stream of ionic liquid into the internal carotid artery during bipolar radiofrequency carotid body ablation.

An apparatus for ablation of perivascular tissue has been conceived comprising a catheter configured for vascular use with a radiofrequency electrode mounted in the vicinity of the distal end connected to one pole of an RF generator, and a mechanical biasing means configured to press the electrode against the medial aspect of the external carotid artery proximate to a target carotid body, and a guidewire, configured for use within the internal carotid artery from an exit point in said catheter proximal to said electrode, and radially aligned with said mechanical biasing means, whereby, said guidewire comprises a hollow structure comprising perforations in the vicinity of its distal end connected to pressurized source of ionic liquid, and a metallic surface associated with said perforations connected to the second pole of said RF generator, wherein, said guidewire is configured, in conjunction with said source of pressurized ionic liquid to elude a stream of ionic liquid into the internal carotid artery during bipolar radiofrequency carotid body ablation.

A method has been conceived for ablating perivascular tissue comprising inserting the distal end of an ablation catheter into the blood vessel of a patient, with the ablation catheter comprising a hollow cylindrical structure with at least one lateral fenestration disposed in the vicinity of the distal end of the catheter shaft, an electrode disposed within the hollow cylindrical structure connected to an electrical connector disposed in the vicinity of the proximal end of the catheter shaft, a fluid channel within the catheter shaft in communication with the interior of the hollow cylindrical structure and a fluid connector disposed in the vicinity of the proximal end of the catheter shaft, where the entire external surface of the catheter assembly is electrically isolated from the electrode; and, connecting the ablation catheter to a source of RF energy and a source of ionic liquid; and, advancing the distal end of the ablation catheter proximate to the perivascular ablation target; then, pressing the lateral fenestration against the wall of the blood vessel oriented towards the perivascular ablation target; then, delivering an ionic liquid to the hollow cylindrical structure in a substantially continuous manner while applying RF energy to the electrode at an energy level and duration sufficient for ablation of the target perivascular tissue, whereby the ionic liquid substantially displaces blood from the space between the vascular wall and the electrode, while conducting RF energy between the vascular wall and the electrode through the vascular wall surface defined by the fenestration.

A method has been conceived for ablating perivascular tissue comprising inserting the distal end of an ablation catheter into the blood vessel of a patient, with the ablation catheter comprising a hollow cylindrical structure with at least one lateral fenestration disposed in the vicinity of the distal end of the catheter shaft, an electrode disposed within the hollow cylindrical structure connected to an electrical connector disposed in the vicinity of the proximal end of the catheter shaft, a fluid channel within the catheter shaft in communication with the interior of the hollow cylindrical structure and a fluid connector disposed in the vicinity of the proximal end of the catheter shaft, a mechanism for pressing the at least one lateral fenestration against a vascular wall that provides the user with a substantially unambiguous fluoroscopic indication of the position of the at least one lateral fenestration within a vascular structure, where the entire external surface of the catheter assembly is electrically isolated from the electrode; and, connecting the ablation catheter to a source of RF energy and a source of ionic liquid; and, advancing the distal end of the ablation catheter proximate to the perivascular ablation target; then, pressing the lateral fenestration against the wall of the blood vessel oriented towards the perivascular ablation target; then, delivering an ionic liquid to the hollow cylindrical structure in a substantially continuous manner while applying RF energy to the electrode at an energy level and duration sufficient for ablation of the target perivascular tissue, whereby the ionic liquid substantially displaces blood from the space between the vascular wall and the electrode, while conducting RF energy between the vascular wall and the electrode through the vascular wall surface defined by the fenestration.

A method has been conceived for ablating perivascular tissue comprising inserting the distal end of an ablation catheter into the blood vessel of a patient, with the ablation catheter comprising an inflatable structure with at least one lateral fenestration disposed in the vicinity of the distal end of the catheter shaft, an electrode disposed within the inflatable structure connected to an electrical connector disposed in the vicinity of the proximal end of the catheter shaft, a fluid channel within the catheter shaft in communication with the interior of the inflatable structure and a fluid connector disposed in the vicinity of the proximal end of the catheter shaft, where the entire external surface of the catheter assembly is electrically isolated from the electrode; and, connecting the ablation catheter to a source of RF energy and a source of ionic liquid; and, advancing the distal end of the ablation catheter proximate to the perivascular ablation target; then, pressing the lateral fenestration against the wall of the blood vessel oriented towards the perivascular ablation target; then, delivering an ionic liquid to the inflatable structure in a substantially continuous manner while applying RF energy to the electrode at an energy level and duration sufficient for ablation of the target perivascular tissue, whereby the ionic liquid substantially displaces blood from the space between the vascular wall and the electrode, while conducting RF energy between the vascular wall and the electrode through the vascular wall surface defined by the fenestration.

A method has been conceived for ablating perivascular tissue comprising inserting the distal end of an ablation catheter into the blood vessel of a patient, with the ablation catheter comprising an inflatable structure with at least one lateral fenestration disposed in the vicinity of the distal end of the catheter shaft, an electrode disposed within the inflatable structure connected to an electrical connector disposed in the vicinity of the proximal end of the catheter shaft, a fluid channel within the catheter shaft in communication with the interior of the inflatable structure and a fluid connector disposed in the vicinity of the proximal end of the catheter shaft, a mechanism for pressing the at least one lateral fenestration against a vascular wall that provides the user with a substantially unambiguous fluoroscopic indication of the position of the at least one lateral fenestration within a vascular structure, where the entire external surface of the catheter assembly is electrically isolated from the electrode; and, connecting the ablation catheter to a source of RF energy and a source of ionic liquid; and, advancing the distal end of the ablation catheter proximate to the perivascular ablation target; then, pressing the lateral fenestration against the wall of the blood vessel oriented towards the perivascular ablation target; then, delivering an ionic liquid to the hollow cylindrical structure in a substantially continuous manner while applying RF energy to the electrode at an energy level and duration sufficient for ablation of the target perivascular tissue, whereby the ionic liquid substantially displaces blood from the space between the vascular wall and the electrode, while conducting RF energy between the vascular wall and the electrode through the vascular wall surface defined by the fenestration.

A method has been conceived for ablating carotid body function comprising inserting the distal end of an ablation catheter into an artery of a patient, with the ablation catheter comprising forceps mechanism disposed in the vicinity of the distal end of the catheter shaft, with at least one forceps element comprising a hollow cylindrical structure with at least one lateral fenestration oriented in the direction of the opposing forceps element, an electrically isolative exterior surface, an interior electrode surface, an electrical connection between the interior electrode surface and an electrical connector disposed in the vicinity of the proximal end of the catheter shaft, a fluid channel within the catheter shaft in communication with the interior of the hollow cylindrical structure and a fluid connector disposed in the vicinity of the proximal end of the catheter shaft; and, connecting the ablation catheter to a source of RF energy, and a source of ionic liquid; and, advancing the distal end of the ablation catheter through the patient's arterial system proximate to a carotid bifurcation associated with a carotid body; then, deploying the forceps mechanism and grasping the carotid bifurcation saddle; then delivering ionic liquid to the at least one hollow cylindrical structure in a substantially continuous manner while applying RF energy to the at least one electrode at an energy level and duration sufficient for ablation of carotid boy function, whereby the ionic liquid substantially displaces blood from the space between the vascular wall and the electrode, while conducting RF energy between the vascular wall and the electrode through the vascular wall surface defined by the fenestration.

A method has been conceived for ablating carotid body function comprising inserting the distal end of an ablation catheter into an artery of a patient, with the ablation catheter comprising forceps mechanism disposed in the vicinity of the distal end of the catheter shaft, with at least one forceps element comprising an inflatable structure with at least one lateral fenestration oriented in the direction of the opposing forceps element, an electrically isolative exterior surface, an interior electrode surface, an electrical connection between the interior electrode surface and an electrical connector disposed in the vicinity of the proximal end of the catheter shaft, a fluid channel within the catheter shaft in communication with the interior of the inflatable structure and a fluid connector disposed in the vicinity of the proximal end of the catheter shaft; and, connecting the ablation catheter to a source of RF energy, and a source of ionic liquid; and, advancing the distal end of the ablation catheter through the patient's arterial system proximate to a carotid bifurcation associated with a carotid body; then, deploying the forceps mechanism and grasping the carotid bifurcation saddle; then delivering ionic liquid to the at least one inflatable structure in a substantially continuous manner while applying RF energy to the at least one electrode at an energy level and duration sufficient for ablation of carotid boy function, whereby the ionic liquid inflates the inflatable structure, and substantially displaces blood from the space between the vascular wall and the electrode, while conducting RF energy between the vascular wall and the electrode through the vascular wall surface defined by the fenestration.

A method has been conceived for ablating carotid body function comprising inserting the distal end of an ablation catheter into an artery of a patient, with the ablation catheter comprising forceps mechanism disposed in the vicinity of the distal end of the catheter shaft, with at least one forceps element comprising a substantially non-electrically conductive porous structure oriented in the direction of the opposing forceps element, an electrode surface in contact with the porous structure, an electrical connection between the electrode surface and an electrical connector disposed in the vicinity of the proximal end of the catheter shaft, a fluid channel within the catheter shaft in communication with the porous structure and a fluid connector disposed in the vicinity of the proximal end of the catheter shaft; and, connecting the ablation catheter to a source of RF energy, and a source of ionic liquid; and, advancing the distal end of the ablation catheter through the patient's arterial system proximate to a carotid bifurcation associated with a carotid body; then, deploying the forceps mechanism and grasping the carotid bifurcation saddle; then delivering ionic liquid to the at least one porous structure in a substantially continuous manner while applying RF energy to the at least one electrode surface at an energy level and duration sufficient for ablation of carotid boy function, whereby the ionic liquid substantially displaces blood from the space between the vascular wall and the electrode, while conducting RF energy between the vascular wall and the electrode through the vascular wall surface in contact with the porous structure.

A method has been conceived for ablating perivascular tissue comprising inserting the distal end of an ablation catheter into the blood vessel of a patient, with the ablation catheter comprising a hollow cylindrical structure with at least one lateral fenestration disposed in the vicinity of the distal end of the catheter shaft, a Piezo-electric element disposed within the hollow cylindrical structure connected to an electrical connector disposed in the vicinity of the proximal end of the catheter shaft by a coaxial cable, a fluid channel within the catheter shaft in communication with the interior of the hollow cylindrical structure and a fluid connector disposed in the vicinity of the proximal end of the catheter shaft, where the entire external surface of the catheter assembly is electrically isolated from the Piezo-electric element; and, connecting the ablation catheter to a source of ultrasonic energy and a source of ionic liquid; and, advancing the distal end of the ablation catheter proximate to the perivascular ablation target; then, pressing the lateral fenestration against the wall of the blood vessel oriented towards the perivascular ablation target; then, delivering an ionic liquid to the hollow cylindrical structure in a substantially continuous manner while applying energy to the Piezo-electric element at a frequency, energy level and duration sufficient for ablation of the target perivascular tissue, whereby the ionic liquid substantially displaces blood from the space between the vascular wall and the Piezo-electric element, while applying ultrasonic energy to the vascular wall through the vascular wall surface defined by the fenestration.

A method has been conceived for ablating perivascular tissue comprising inserting the distal end of an ablation catheter into the blood vessel of a patient, with the ablation catheter comprising a hollow cylindrical structure with at least one lateral fenestration disposed in the vicinity of the distal end of the catheter shaft, a Piezo-electric element disposed within the hollow cylindrical structure connected to an electrical connector disposed in the vicinity of the proximal end of the catheter shaft by a coaxial cable, a fluid channel within the catheter shaft in communication with the interior of the hollow cylindrical structure and a fluid connector disposed in the vicinity of the proximal end of the catheter shaft, a mechanism configured for pressing the at least one lateral fenestration against a vascular wall disposed in the vicinity of the hollow cylindrical structure which provides a user with a substantially unambiguous fluoroscopic indication of the position of the at least one lateral fenestration within a vascular structure, where the entire external surface of the catheter assembly is electrically isolated from the Piezo-electric element; and, connecting the ablation catheter to a source of ultrasonic energy and a source of ionic liquid; and, advancing the distal end of the ablation catheter proximate to the perivascular ablation target; then, pressing the lateral fenestration against the wall of the blood vessel oriented towards the perivascular ablation target; then, delivering an ionic liquid to the hollow cylindrical structure in a substantially continuous manner while applying energy to the Piezo-electric element at a frequency, energy level and duration sufficient for ablation of the target perivascular tissue, whereby the ionic liquid substantially displaces blood from the space between the vascular wall and the Piezo-electric element, while applying ultrasonic energy to the vascular wall through the vascular wall surface defined by the fenestration.

A method has been conceived for ablating perivascular tissue comprising inserting the distal end of an ablation catheter into the blood vessel of a patient, with the ablation catheter comprising a hollow cylindrical structure with at least one lateral fenestration disposed in the vicinity of the distal end of the catheter shaft, a Piezo-electric element disposed within the hollow cylindrical structure connected to an electrical connector disposed in the vicinity of the proximal end of the catheter shaft by a coaxial cable, a fluid channel within the catheter shaft in communication with the interior of the hollow cylindrical structure and a fluid connector disposed in the vicinity of the proximal end of the catheter shaft, where the entire external surface of the catheter assembly is electrically isolated from the Piezo-electric element; and, connecting the ablation catheter to a source of ultrasonic energy and a source of ionic liquid; and, advancing the distal end of the ablation catheter proximate to the perivascular ablation target; and administering an ultrasonic contrast agent comprising microbubbles (micro-balloons) trans-venously; and, pressing the lateral fenestration against the wall of the blood vessel oriented towards the perivascular ablation target; then, delivering an ionic liquid to the hollow cylindrical structure in a substantially continuous manner; applying a pulse of ultrasonic energy to the Piezo-electric at a frequency and mechanical index sufficient to stimulate contrast enhanced harmonic emissions in the target perivascular tissue, and measuring the level and frequency distributions of the harmonic emissions using the Piezo-electric element; then, activating the Piezo-electric element at a level, frequency, and duration sufficient for ultrasonic ablation of the target perivascular tissue; then, applying a pulse of ultrasonic energy to the Piezo-electric at a frequency and mechanical index sufficient to stimulate contrast enhanced harmonic emissions in the target perivascular tissue, and measuring the level and frequency distributions of the harmonic emissions using the Piezo-electric element; then, determining the effectiveness of the ultrasonic ablation by comparing the measured harmonic emissions prior to the ablation to the harmonic emissions following the ablation, whereby, the ionic liquid substantially displaces blood and ultrasonic contrast agent from the space between the vascular wall and the Piezo-electric element, while ultrasonic energy is directed to the vascular wall surface through the fenestration.

A method has been conceived for ablating perivascular tissue comprising inserting the distal end of an ablation catheter into the blood vessel of a patient, with the ablation catheter comprising a hollow cylindrical structure with at least one lateral fenestration disposed in the vicinity of the distal end of the catheter shaft, a Piezo-electric element disposed within the hollow cylindrical structure connected to an electrical connector disposed in the vicinity of the proximal end of the catheter shaft by a coaxial cable, an RF electrode surface disposed within the hollow cylindrical structure connected to an electrical connector disposed in the vicinity of the proximal end of the catheter shaft, a fluid channel within the catheter shaft in communication with the interior of the hollow cylindrical structure and a fluid connector disposed in the vicinity of the proximal end of the catheter shaft, a mechanism configured for pressing the at least one lateral fenestration against a vascular wall disposed in the vicinity of the hollow cylindrical structure which provides a user with a substantially unambiguous fluoroscopic indication of the position of the at least one lateral fenestration within a vascular structure, where the entire external surface of the catheter assembly is electrically isolated from the Piezo-electric element; and, connecting the ablation catheter to a source of ultrasonic energy, a source of RF energy, and a source of ionic liquid; and, advancing the distal end of the ablation catheter proximate to the perivascular ablation target; then, pressing the lateral fenestration against the wall of the blood vessel oriented towards the perivascular ablation target; then, delivering an ionic liquid to the hollow cylindrical structure in a substantially continuous manner while applying energy to the Piezo-electric element and the RF electrode surface at a frequency, energy level and duration sufficient for ablation of the target perivascular tissue, whereby the ionic liquid substantially displaces blood from the space between the vascular wall and the Piezo-electric element, while applying ultrasonic and RF energy to the vascular wall through the vascular wall surface defined by the fenestration.

A method has been conceived for ablation of carotid body function comprising inserting a sheath into a common carotid artery in an antegrade direction, then deploying a first catheter into the associated external carotid artery from said sheath, and deploying a second catheter into the associated internal carotid artery from said sheath, whereby, said first catheter comprises a radiofrequency electrode connectable to one pole of an RF generator, and a mechanical biasing means configured to press the electrode against the medial aspect of the external carotid artery proximate to a target carotid body, and whereby said second catheter comprises a perforated non-conductive balloon housing an RF electrode connectable to the second pole of said RF generator, with the interior of said balloon being fluidically connectable to a pressurized source of ionic liquid, then connecting said first catheter electrode to one pole of an RF generator, connecting said second catheter electrode to the second pole of said RF generator, and connecting said perforated balloon to a source of pressurized ionic liquid causing said perforated balloon to elude a stream of ionic liquid into the internal carotid artery, then activating said RF generator at a level, and for a duration determined sufficient to ablate carotid body function.

A method has been conceived for ablation of carotid body function comprising inserting a sheath into a common carotid artery in an antegrade direction, then deploying a first catheter into the associated external carotid artery from said sheath, and deploying a bladder against the medial aspect of the proximal internal carotid artery from said sheath, whereby, said first catheter comprises a radiofrequency electrode connectable to one pole of an RF generator, and a mechanical biasing means configured to press the electrode against the medial aspect of the external carotid artery proximate to a target carotid body, and whereby said bladder comprises a perforated non-conductive membranous structure housing an RF electrode connectable to the second pole of said RF generator, with the interior of said bladder being fluidically connectable to a pressurized source of ionic liquid, then connecting said first catheter electrode to one pole of an RF generator, connecting said bladder electrode to the second pole of said RF generator, and connecting said perforated bladder to a source of pressurized ionic liquid causing said perforated bladder to elude a stream of ionic liquid into the internal carotid artery, then activating said RF generator at a level, and for a duration determined sufficient to ablate carotid body function.

A method has been conceived for ablation of carotid body function comprising inserting a catheter into an external carotid artery in an antegrade direction, with said catheter comprising a an electrode connectable to one pole of an RF generator, a mechanical biasing means configured for pressing said electrode against the medial aspect of the proximal external carotid artery proximate to the target carotid body, and a metallic perforated fluid port located proximal to said electrode, and radially aligned with said mechanical biasing means, which is electrically connectable to the second pole of said RF generator, and fluidically connectable to a pressurized source of ionic liquid, then connecting said catheter electrode to one pole of an RF generator, connecting said metallic perforated fluid port to the second pole of said RF generator, and connecting said metallic perforated fluid port to a source of pressurized ionic liquid causing said metallic perforated fluid port to elude a stream of ionic liquid into the internal carotid artery, then activating said RF generator at a level, and for a duration determined sufficient to ablate carotid body function.

A method has been conceived for ablation of carotid body function comprising inserting a catheter into an external carotid artery in an antegrade direction, with said catheter comprising a an electrode connectable to one pole of an RF generator, a mechanical biasing means configured for pressing said electrode against the medial aspect of the proximal external carotid artery proximate to the target carotid body, and a guidewire configured for use within the associated internal carotid artery from an exit port in said catheter proximal to said electrode and radially aligned with said mechanical biasing means, with said guidewire comprising a hollow structure comprising fenestrations in the vicinity of the distal end, connectable to a source pressurized ionic liquid, and a metallic surface associated with said hollow structure connectable to the second pole of said RF generator, then connecting said catheter electrode to one pole of an RF generator, connecting said metallic surface associated with said hollow structure to the second pole of said RF generator, and connecting said hollow structure to a source of pressurized ionic liquid causing said guidewire to elude a stream of ionic liquid into the internal carotid artery, then activating said RF generator at a level, and for a duration determined sufficient to ablate carotid body function.

A kit for ablation of carotid body function in a patient has been conceived comprising an ablation catheter comprising a thermal ablation element, comprising a hollow structure, at least one lateral fenestration, with an electrically insulative outer surface, an electrically conductive inner surface connectable to an electrical energy source, and a means to connect the interior of the hollow structure to a source of ionic liquid mounted in the vicinity of the distal end, a catheter shaft with a caliber between 3 French and 6 French, with a working length between 15 cm and 25 cm, a mechanism configured for positioning the thermal ablation element against the wall of a carotid artery adjacent to a carotid body, a mechanism for providing the user with a substantially unambiguous fluoroscopic indication of the position of the thermal ablation element within an external carotid artery, and a means for connecting the thermal ablation element to a source of thermal ablation energy mounted in the vicinity of the proximal end; an arterial access sheath configured for superficial temporal artery access comprising a hollow thin walled tubular structure sized to accommodate a 3 French to 6 French ablation catheter internally, with a working length between 10 cm and 20 cm, a radiopaque marker in the vicinity of the distal end of the tubular structure, and a valve and a fluid port mounted in the vicinity of the proximal end; and, instructions for use comprising instructions for accessing a superficial temporal artery in a retrograde manner, and positioning the ablation catheter for ablation of carotid body function.

Placing the ablation element (e.g. radiofrequency electrode) at a suitable location for carotid body modulation may be facilitated by a structure at a distal region of an ablation device (e.g. endovascular catheter) that comprises two arms configured to couple with a carotid bifurcation. The structure comprising two arms may comprise an ablation element on one arm or an ablation element on each of the two arms, or multiple ablation elements on one or each of the arms. The ablation element(s) may be positioned on the arms such that when the structure is coupled to a carotid bifurcation the ablation elements are placed at a suitable location (e.g. at or between about 0 to 15 mm, 4 to 15 mm, or 4 to 10 mm from a carotid bifurcation on an inner wall of an external carotid artery and internal carotid artery and within a vessel wall arc having an arc length of about 25% of the vessel circumference facing the opposing ablation element) on a target ablation site for effective carotid body modulation. The structure may further facilitate apposition of ablation element(s) with tissue.

In another exemplary procedure a location of periarterial space associated with a carotid body is identified, then an ablation element is placed against the interior wall of a carotid artery adjacent to the identified location, then ablation parameters are selected and the ablation element is activated thereby ablating the carotid body, whereby the position of the ablation element and the selection of ablation parameters provides for ablation of the carotid body without substantial collateral damage to adjacent functional structures.

In further example the location of the periarterial space associated with a carotid body is identified, as well as the location of important non-target nerve structures not associated with the carotid body, then an ablation element is placed against the interior wall of a carotid artery adjacent to the identified location, ablation parameters are selected and the ablation element is then activated thereby ablating the carotid body, whereby the position of the ablation element and the selection of ablation parameters provides for ablation of the target carotid body without substantial collateral damage to important non-target nerve structures in the vicinity of the carotid body.

Selectable carotid body modulation parameters may include ablation element temperature, duration of ablation element activation, ablation power, ablation element force of contact with a vessel wall, ablation element size, ablation modality, and ablation element position within a vessel.

The location of the perivascular space associated with a carotid body may be determined by means of a non-fluoroscopic imaging procedure prior to carotid body modulation, where the non-fluoroscopic location information is translated to a coordinate system based on fluoroscopically identifiable anatomical and/or artificial landmarks.

A function of a carotid body may be stimulated (e.g. excited with electric signal or chemical) and at least one physiological parameter is recorded prior to and during the stimulation, then the carotid body is ablated, and the stimulation is repeated, whereby the change in recorded physiological parameter(s) prior to and after ablation is an indication of the effectiveness of the ablation.

A function of a carotid body may be temporarily blocked and at least one physiological parameter(s) is recorded prior to and during the blockade, then the carotid body is ablated, and the blockade is repeated, whereby the change in recorded physiological parameter(s) prior to and after ablation is an indication of the effectiveness of the ablation.

A device configured to prevent embolic debris from entering the brain may be deployed in an internal carotid artery associated with a carotid body, then an ablation element is placed within and against the wall of an external carotid artery or an internal carotid artery associated with the carotid body, the ablation element is activated resulting in carotid body modulation, the ablation element is then withdrawn, then the embolic prevention device is withdrawn, whereby the embolic prevention device in the internal carotid artery prevents debris resulting from the use of the ablation element form entering the brain.

A method has been conceived in which the location of the perivascular space associated with a carotid body is identified, then an ablation element is placed in a predetermined location against the interior wall of vessel adjacent to the identified location, then ablation parameters are selected and the ablation element is activated and then deactivated, the ablation element is then repositioned in at least one additional predetermine location against the same interior wall and the ablation element is then reactivated using the same or different ablation parameters, whereby the positions of the ablation element and the selection of ablation parameters provides for ablation of the carotid body without substantial collateral damage to adjacent functional structures.

A system has been conceived comprising a vascular catheter with an ablation element mounted in the vicinity of a distal end configured for tissue heating, whereby, the ablation element comprises at least one electrode and at least one temperature sensor, a connection between the ablation element electrode(s) and temperature sensor(s) to an ablation energy source, with the ablation energy source being configured to maintain the ablation element at a temperature in the range of 36 to 100 degrees centigrade during ablation using signals received from the temperature sensor(s). For example, in an embodiment the at least one ablation element in contact with blood is maintained at a temperature between 36 and 50 degrees centigrade to minimize coagulation while targeted periarterial tissue is heated to a temperature between 50 and 100 degrees centigrade to ablate tissue but avoid boiling of water and steam and gas expansion in the tissue.

### BRIEF DESCRIPTION OF THE DRAWINGS

FIG. 1A is a schematic illustration of a patient in supine position depicting vascular access to the region of a target carotid body using a femoral artery puncture.

FIG. 1B is a schematic illustration of carotid vasculature showing an intercarotid septum.

FIG. 1C is a schematic illustration of a transverse cross-section of an intercarotid septum.

FIG. 2 is an illustration of the side of a patient's head depicting vascular access to the region of a carotid body using a superficial temporal artery puncture.

FIG. 3A is an illustration of the distal end of a carotid body ablation catheter that utilizes an RF tissue contact electrode comprising a stream of ionic liquid.

FIG. 3B is an illustration of the distal end of a carotid body ablation catheter in exploded view that utilizes an RF tissue contact electrode comprising a stream of ionic liquid.

FIG. 4A is an illustration of the distal end of a carotid body ablation forceps catheter that utilizes an RF tissue contact electrode comprising a stream of ionic liquid in insertion and navigation configuration.

FIG. 4B is an illustration of the carotid body ablation forceps catheter with the forceps deployed.

FIG. 5 is an illustration of the distal end of a carotid body ablation catheter with a side-port guidewire configured for use in a carotid bifurcation that utilizes an RF tissue contact electrode comprising a stream of ionic liquid.

FIG. 6A is an illustration of the distal end of a carotid body ablation balloon catheter that utilizes an RF tissue contact electrode comprising a stream of ionic liquid showing the balloon deflated.

FIG. 6B is an illustration of the distal end of a carotid body ablation balloon catheter that utilizes an RF tissue contact electrode comprising a stream of ionic liquid showing the balloon inflated.

FIG. 7 is an illustration of the distal end of a carotid body ablation catheter that utilizes an RF tissue contact electrode comprising a stream of ionic liquid where the liquid flows through a slit in an elastomeric membrane.

FIG. 8A is an isometric front view illustration of the distal end of a carotid body ablation catheter configured for use by trans-superficial temporal artery access to the region of a target carotid body and transmural ablation from within an external carotid artery.

FIG. 8B is an isometric rear view illustration of the carotid body ablation catheter configured for trans-superficial temporal artery access showing a push wire retracted.

FIG. 8C is an isometric rear view illustration of the carotid body ablation catheter configured for trans-superficial temporal artery access showing a push wire extended.

FIG. 9 is an illustration of a procedure kit for trans-temporal artery ablation of a carotid body, comprising a puncture needle, guidewire, arterial sheath and obturator, and a carotid body ablation catheter that utilizes an RF tissue contact electrode comprising a stream of ionic liquid.

FIG. 10A is an illustration of the distal end of a dual mode RF/ultrasonic carotid body ablation catheter configured for use in conjunction with systemically administered ultrasonic contrast agent that utilizes a stream of ionic liquid.

FIG. 10B is an exploded view illustration of the front of the distal end of a dual mode RF/ultrasonic carotid body ablation catheter.

FIG. 10C is an exploded view illustration of the rear of the distal end of a dual mode RF/ultrasonic carotid body ablation catheter.

FIG. 11 is a schematic illustration of a carotid body ablation forceps catheter in situ during a carotid body ablation utilizing an ionic liquid stream.

FIG. 12 is a schematic illustration of a carotid body ablation catheter with a side-port guidewire in situ during a carotid body ablation utilizing an ionic liquid stream.

FIG. 13 is a schematic illustration of a carotid body ablation balloon catheter in situ during a carotid body ablation utilizing an ionic liquid stream.

FIG. 14 is a schematic illustration of a carotid body ablation catheter with an elastomeric membrane comprising a slit in situ during a carotid body ablation.

FIG. 15 is a schematic illustration of a carotid body ablation catheter in situ utilizing access to the region of the carotid body from a superficial temporal artery during a carotid body ablation utilizing an ionic liquid stream.

FIG. 16 is an illustration of a carotid body ablation system configured for carotid body ablation utilizing an ionic liquid stream.

FIG. 17 is a graph of primary and harmonic acoustic intensities prior to, and after a successful carotid body ablation with a dual mode RF/ultrasonic carotid body ablation catheter utilizing an ionic liquid stream.

FIG. 18 is a graph of primary and harmonic acoustic intensities during a carotid body ablation with a dual mode RF/ultrasonic carotid body ablation catheter.

FIG. 19 is a schematic illustration of a bipolar, bifurcated carotid body ablation catheter with one arm comprising a metallic RF electrode configured for use in an external carotid artery, and the second arm comprising a perforated balloon configured as the second electrode for use in an internal carotid artery, which utilizes an ionic liquid stream.

FIG. 20 is an in situ schematic illustration of a bipolar, bifurcated carotid body ablation catheter with one arm comprising a metallic RF electrode, and a second arm comprising a perforated balloon configured as the second electrode, which utilizes and ionic liquid stream.

FIG. 21 is an in situ schematic illustration of a bipolar, carotid body ablation catheter with one electrode comprising a metallic RF electrode configured for use in an external carotid artery, and the second electrode comprising a perforated bladder configured as the second electrode for use against the medial aspect of a proximal internal carotid artery, which utilizes an ionic liquid stream.

FIG. 22 is an in situ schematic illustration of a bipolar, carotid body ablation catheter comprising a metallic RF electrode configured for use in an external carotid artery, and a second electrode comprising a perforated proximal electrode configured to infuse an ionic liquid stream into an internal carotid artery from the region of the distal common carotid artery.

FIG. 23 is an in situ schematic illustration of a bipolar, bifurcated carotid body ablation catheter with one arm comprising a metallic RF electrode configured for use in an external carotid artery, and the second arm comprising a perforated guidewire configured as the second electrode for use in an internal carotid artery, which utilizes an ionic liquid stream.

### DETAILED DESCRIPTION

In the following detailed description, reference is made to the accompanying drawings, which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized and that structural, logical and electrical changes may be made without departing from the spirit and scope of the present invention.

References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

Systems, devices, and methods have been conceived for carotid body ablation (that is, to ablate fully or partially one or both carotid bodies, carotid body nerves, intercarotid septums, or peripheral chemoreceptors) to treat patients having a sympathetically mediated disease (e.g. cardiac, renal, metabolic, or pulmonary disease such as hypertension, CHF, sleep apnea, sleep disordered breathing, diabetes, insulin resistance, atrial fibrillation, chronic kidney disease, polycystic ovarian syndrome, post MI mortality) at least partially resulting from augmented peripheral chemoreflex (e.g. peripheral chemoreceptor hypersensitivity, peripheral chemosensor hyperactivity), heightened sympathetic activation, or an unbalanced autonomic tone.

A reduction of peripheral chemoreflex or reduction of afferent nerve signaling from at least one carotid body (CB) resulting in a reduction of central sympathetic tone is a main therapy pathway. Higher than normal chronic or intermittent activity of afferent carotid body nerves is considered enhanced chemoreflex. Other therapeutic benefits such as increase of parasympathetic tone, vagal tone and specifically baroreflex and baroreceptor activity, as well as reduction of dyspnea, hyperventilation, hypercapnea, respiratory alkalosis and breathing rate may be expected in some patients. Secondary to reduction of breathing rate additional increase of parasympathetic tone can be expected in some patients. Reduced breathing rate can lead to increased tidal lung volume, reduced dead space and increased efficiency of gas exchange. Reduced dyspnea and reduced dead space can independently lead to improved ability to exercise. Shortness of breath (dyspnea) and exercise limitations are common debilitating symptoms in CHF and COPD. Augmented peripheral chemoreflex (e.g. carotid body activation) leads to increases in sympathetic nervous system activity, which is in turn primarily responsible for the progression of chronic disease as well as debilitating symptoms and adverse events seen in our intended patient populations. Carotid bodies contain cells that are sensitive to partial pressure of oxygen and carbon dioxide in blood plasma. Carotid bodies also may respond to blood flow, pH acidity, glucose level in blood and possibly other variables. Thus carotid body modulation may be a treatment for patients, for example having hypertension, heart disease or diabetes, even if chemosensitive cells are not activated.

An inventive treatment, endovascular transmural carotid body ablation (also herein referred to as carotid body modulation) is disclosed that may involve inserting a catheter in the patient's vascular system, positioning a distal region of the catheter in a vessel proximate a carotid body (e.g. in a common carotid artery, internal carotid artery, external carotid artery, at a carotid bifurcation, proximate an intercarotid septum, in an artery or vein proximate an intercarotid septum), positioning an ablation element proximate to a target site (e.g. a carotid body, afferent nerves associated with a carotid body, a peripheral chemosensor, an intercarotid septum), and delivering an ablation agent from the ablation element to ablate the target site. Several methods and devices for carotid body ablation are described.

Some patients suffering from a sympathetically mediated disease who may benefit from a carotid body ablation procedure may have a significant amount of atheromatous plaque in their carotid arteries. Performing an endovascular procedure in the presence of plaque may pose a risk of brain embolism, particularly if the plaque is in the internal carotid artery, which feeds the brain, and the endovascular procedure involved significant mechanical manipulation in the internal carotid artery. Therefore, there may be a reduced risk benefit of an endovascular catheter configured to ablate a carotid body while minimizing mechanical manipulation or contact forces on a carotid artery wall or in association with plaque. Endovascular catheters have been conceived comprising a virtual electrode, that is, an electrode that delivers ablative energy via an ionic liquid stream, which may reduce mechanical manipulation or contact forces on a carotid artery wall or in association with plaque.

A bipolar radiofrequency arrangement for carotid body ablation, wherein a first electrode is placed in an external carotid artery and a second electrode is placed in an internal carotid artery and radiofrequency electrical current is passed from the first electrode through a carotid septum to the second electrode, is found by the inventors to have benefits of creating a well controlled ablation that is significantly large to effectively ablate a target site (e.g. a carotid body, carotid body nerves, a portion of a carotid body sufficient to cause a therapeutic effect) and that is contained within safe margins to avoid important non-target nerves and organs. A virtual electrode may be used in a bipolar arrangement wherein the virtual electrode may be placed in the internal carotid artery, the external carotid artery, or both to reduce a risk of plaque dislodgement.

Endovascular access for a carotid body ablation procedure may involve passing a catheter through a tortuous vessel pathway. For example, endovascular access to a carotid artery via femoral artery introduction requires traversing tortuous bends in an aortic arch. Furthermore, endovascular access may also require passing a catheter through a narrow vessel. For example, carotid arteries may have a diameter between about 4 and 8 mm. Carotid artery access via a superficial temporal artery requires passing through an artery that may have a diameter of about 3 mm. Therefore, a catheter configured for endovascular carotid body ablation may require a small diameter, for example less than about 3 mm or about 2 mm. Thus, an electrode size may be limited as well. A virtual electrode may allow a larger joule effect zone than a solid electrode to create a larger ablation than that created by a diameter-limited solid electrode.

### Targets:

To inhibit or suppress a peripheral chemoreflex, anatomical targets for ablation (also referred to as targeted tissue, target ablation sites, or target sites) may include at least a portion of at least one carotid body, an aortic body, nerves associated with a peripheral chemoreceptor (e.g. carotid body nerves, carotid sinus nerve, carotid plexus), small blood vessels feeding a peripheral chemoreceptor, carotid body parenchyma, chemosensitive cells (e.g. glomus cells), tissue in a location where a carotid body is suspected to reside (e.g. a location based on pre-operative imaging or anatomical likelihood), an intercarotid septum, a portion of an intercarotid septum or a combination thereof. As used herein, ablation of a carotid body or carotid body modulation may refer to ablation of any of these target ablation sites.

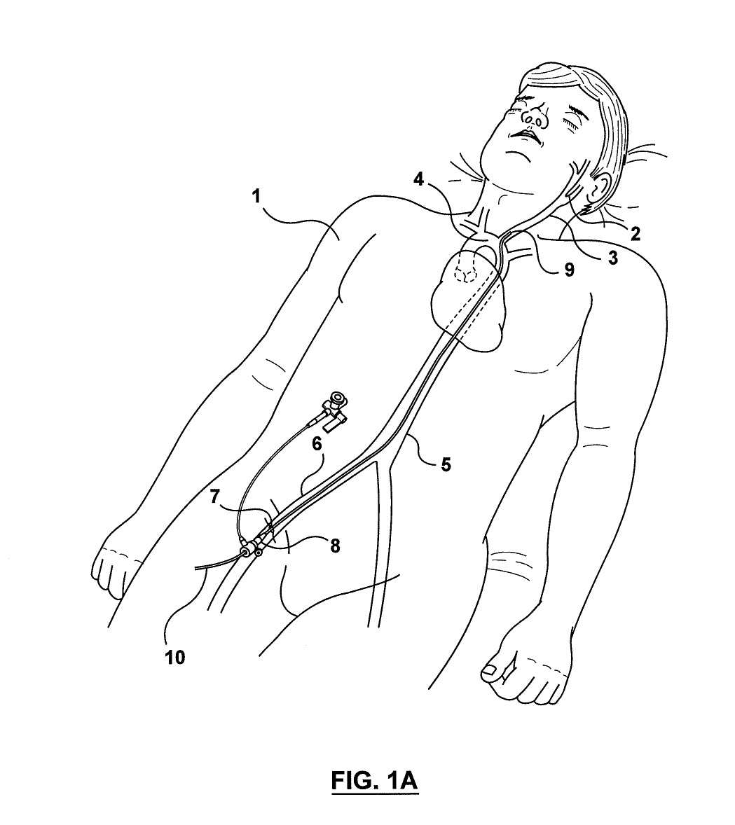

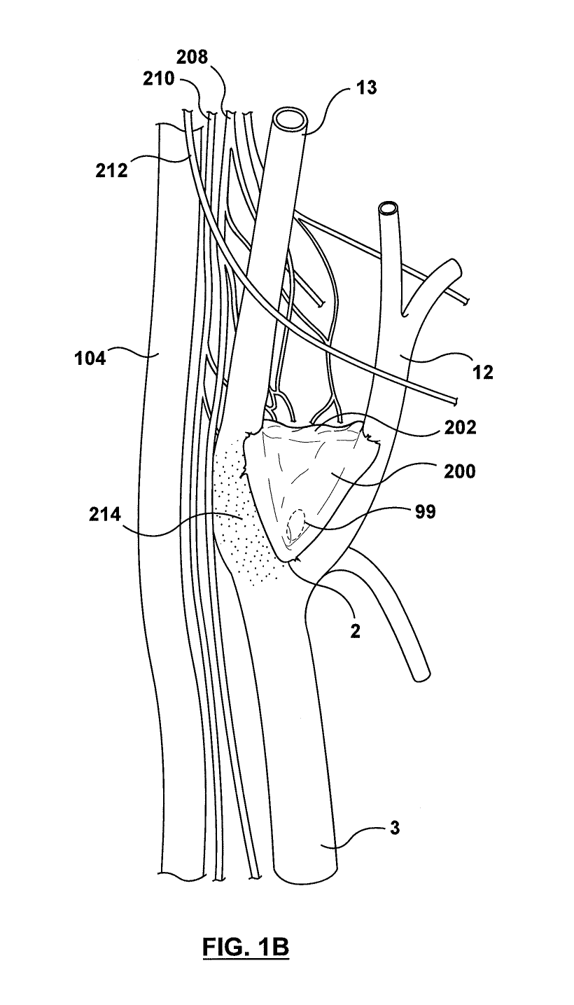

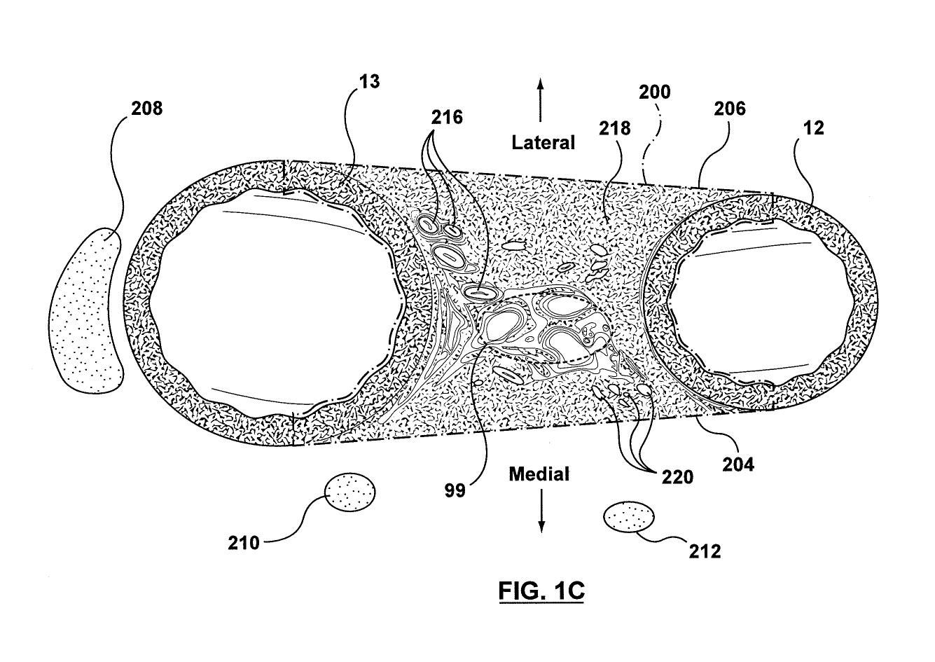

Shown in FIG. 1B, a carotid body (CB) 99, housing peripheral chemoreceptors, modulates sympathetic tone through direct signaling to the central nervous system. Carotid bodies represent a paired organ system located near a bifurcation 2 of a common carotid artery 3 bilaterally, that is, on both sides of the neck. The common carotid artery 3 bifurcates to an internal carotid artery 13 and an external carotid artery 12. Typically, in humans each carotid body is approximately the size of a 2.5-5 mm ovoid grain of rice and is innervated both by the carotid sinus nerve (CSN, a branch of the glossopharyngeal nerve), and the ganglioglomerular (sympathetic) nerve of the nearby superior cervical ganglion. Infrequently other shapes are encountered. The CB is the most perfused organ per gram weight in the body and receives blood via an arterial branch or branches typically arising from internal or external carotid artery.

An intercarotid septum 200 (also referred to as carotid septum) shown in FIGS. 1B and 1C is herein defined as a wedge or triangular segment of tissue with the following boundaries: A saddle of a carotid bifurcation 2 defines a caudal aspect (an apex) of a carotid septum 200; Facing walls of internal 13 and external 12 carotid arteries define two sides of a carotid septum; A cranial boundary 202 of a carotid septum extends between these arteries and may be defined as cranial to a carotid body but caudal to any important non-target nerve structures (e.g. hypoglossal nerve) that might be in the region, for example a cranial boundary may be about 7 mm to 15 mm (e.g. about 10 mm) from the saddle of the carotid bifurcation; Medial 204 and lateral 206 walls of the carotid septum 200 are generally defined by planes approximately tangent to the internal and external carotid arteries; One of the planes is tangent to the lateral wall of the internal and external carotid arteries and the other plane is tangent to the medial walls of these arteries. An intercarotid septum is between the medial and lateral walls. The medial plane of an intercarotid septum may alternatively be defined as a carotid sheath on a medial side of a septum or within about 2 mm outside of the medial side of the carotid sheath. An intercarotid septum 200 may contain a carotid body 99 and is typically absent of important non-target nerve structures such as a vagus nerve 208, important non-target sympathetic nerves 210, or a hypoglossal nerve 212. Therefor, creating an ablation that is maintained within an intercarotid septum may effectively modulate a carotid body while safely avoiding collateral damage of important non-target nerve structures. An intercarotid septum may include some baroreceptors 214 or baroreceptor nerves. An intercarotid septum may also include small blood vessels 216, nerves 220 associated with the carotid body, and fat 218.

Carotid body nerves are anatomically defined herein as carotid plexus nerves 220 and carotid sinus nerves. Carotid body nerves are functionally defined herein as nerves that conduct information from a carotid body to a central nervous system.

An ablation may be focused exclusively on targeted tissue, or be focused on the targeted tissue while safely ablating tissue proximate to the targeted tissue (e.g. to ensure the targeted tissue is ablated or as an approach to gain access to the targeted tissue). An ablation may be as big as a peripheral chemoreceptor (e.g. carotid body or aortic body) itself, somewhat smaller, or bigger and can include tissue surrounding the chemoreceptor such as blood vessels, adventitia, fascia, small blood vessels perfusing the chemoreceptor, or nerves connected to and innervating the glomus cells. An intercarotid plexus or carotid sinus nerve maybe a target of ablation with an understanding that some baroreceptor nerves will be ablated together with carotid body nerves. Baroreceptors are distributed in the human arteries and have high degree of redundancy.

Tissue may be ablated to inhibit or suppress a chemoreflex of only one of patient's two carotid bodies. Alternatively, a carotid body modulation procedure may involve ablating tissue to inhibit or suppress a chemoreflex of both of a patient's carotid bodies. For example a therapeutic method may include ablation of one carotid body, measurement of resulting chemosensitivity, sympathetic activity, respiration or other parameter related to carotid body hyperactivity and ablation of the second carotid body if needed to further reduce chemosensitivity following unilateral ablation. The decision to ablate one or both carotid bodies may be based on pre-procedure testing or on patient's anatomy.

An embodiment of a therapy may substantially reduce chemoreflex without excessively reducing the baroreflex of the patient. The proposed ablation procedure may be targeted to substantially spare the carotid sinus, baroreceptors distributed in the walls of carotid arteries (e.g. internal carotid artery), and at least some of the carotid sinus baroreceptor nerves that conduct signals from said baroreceptors. For example, the baroreflex may be substantially spared by targeting a limited volume of ablated tissue possibly enclosing the carotid body, tissues containing a substantial number of carotid body nerves, tissues located in periadventitial space of a medial segment of a carotid bifurcation, or tissue located at the attachment of a carotid body to an artery. Said targeted ablation is enabled by visualization of the area or carotid body itself, for example by CT, CT angiography, MRI, ultrasound sonography, IVUS, OCT, intracardiac echocardiography (ICE), trans-esophageal echocardiography (TEE), fluoroscopy, blood flow visualization, or injection of contrast, and positioning of an instrument in the carotid body or in close proximity while avoiding excessive damage (e.g. perforation, stenosis, thrombosis) to carotid arteries, baroreceptors, carotid sinus nerves or other important non-target nerves such as vagus nerve or sympathetic nerves located primarily outside of the carotid septum. CT angiography and ultrasound sonography have been demonstrated to locate carotid bodies in most patients. Thus imaging a carotid body before ablation may be instrumental in (a) selecting candidates if a carotid body is present, large enough and identified and (b) guiding therapy by providing a landmark map for an operator to guide an ablation instrument to the carotid septum, center of the carotid septum, carotid body nerves, the area of a blood vessel proximate to a carotid body, or to an area where carotid body itself or carotid body nerves may be anticipated. It may also help exclude patients in whom the carotid body is located substantially outside of the carotid septum in a position close to a vagus nerve, hypoglossal nerve, jugular vein or some other structure that can be endangered by ablation. In one embodiment only patients with carotid body substantially located within the intercarotid septum are selected for ablation therapy. Pre-procedure imaging can also be instrumental in choosing the right catheter depending on a patient's anatomy. For example a catheter with more space between arms can be chosen for a patient with a wider septum.

Once a carotid body is ablated, surgically removed, or denervated, the carotid body function (e.g. carotid body chemoreflex) does not substantially return in humans (in humans aortic chemoreceptors are considered undeveloped). To the contrary, once a carotid sinus baroreflex is removed (such as by resection of a carotid sinus nerve) it is generally compensated, after weeks or months, by the aortic or other arterial baroreceptor baroreflex. Thus, if both the carotid chemoreflex and baroreflex are removed or substantially reduced, for example by interruption of the carotid sinus nerve or intercarotid plexus nerves, baroreflex may eventually be restored while the chemoreflex may not. The consequences of temporary removal or reduction of the baroreflex can be in some cases relatively severe and require hospitalization and management with drugs, but they generally are not life threatening, terminal or permanent. Thus, it is understood that while selective removal of carotid body chemoreflex with baroreflex preservation may be desired, it may not be absolutely necessary in some cases.

### Ablation:

The term “ablation” may refer to the act of altering tissue to suppress or inhibit its biological function or ability to respond to stimulation permanently or for an extended period of time (e.g. greater than 3 weeks, greater than 6 months, greater than a year, for several years, or for the remainder of the patient's life). For example, ablation may involve, but is not limited to, thermal necrosis, selective denervation, embolization (e.g. occlusion of blood vessels feeding the carotid body), or artificial sclerosing of blood vessels.

Carotid Body Ablation (CBA), also referred to herein as carotid body modulation, herein refers to ablation of a target tissue wherein the desired effect is to reduce or remove the afferent neural signaling from a chemosensor (e.g. carotid body) or reducing a chemoreflex. Chemoreflex or afferent nerve activity cannot be directly measured in a practical way, thus indexes of chemoreflex such as chemosensitivity can sometimes be used instead. Chemoreflex reduction is generally indicated by a reduction of an increase of ventilation and respiratory effort per unit of blood gas concentration, saturation or blood gas partial pressure change or by a reduction of central sympathetic nerve activity in response to stimulus (such as intermittent hypoxia or infusion of a drug) that can be measured directly. Sympathetic nerve activity can be assessed indirectly by measuring activity of peripheral nerves leading to muscles (MSNA), heart rate (HR), heart rate variability (HRV), production of hormones such as renin, epinephrine and angiotensin, and peripheral vascular resistance. All these parameters are measurable and their change can lead directly to the health improvements. In the case of CHF patients blood pH, blood PCO₂, degree of hyperventilation and metabolic exercise test parameters such as peak VO₂, and VE/VCO₂ slope are also important. It is believed that patients with heightened chemoreflex have low VO₂ and high VE/VCO₂ slope measured during cardiopulmonary stress test (indexes of respiratory efficiency) as a result of, for example, tachypnea and low blood CO₂. These parameters are also related to exercise limitations that further speed up patient's status deterioration towards morbidity and death. It is understood that all these indexes are indirect and imperfect and intended to direct therapy to patients that are most likely to benefit or to acquire an indication of technical success of ablation rather than to proved an exact measurement of effect or guarantee a success. It has been observed that some tachyarrhythmias in cardiac patients are sympathetically mediated. Thus, carotid body modulation may be instrumental in treating reversible atrial fibrillation and ventricular tachycardia.

In the context of this disclosure ablation includes denervation, which means destruction of nerves or their functional destruction, meaning termination of their ability to conduct signals. Selective denervation may involve, for example, interruption of afferent nerves from a carotid body while substantially preserving nerves from a carotid sinus, which conduct baroreceptor signals. Another example of selective denervation may involve interruption of nerve endings terminating in chemo sensitive cells of carotid body, a carotid sinus nerve, or intercarotid plexus which is in communication with both a carotid body and some baroreceptors wherein chemoreflex or afferent nerve stimulation from the carotid body is reduced permanently or for an extended period of time (e.g. years) and baroreflex is substantially restored in a short period of time (e.g. days or weeks). As used herein, the term “ablate” refers to interventions that suppress or inhibit natural chemoreceptor or afferent nerve functioning, which is in contrast to electrically neuromodulating or reversibly deactivating and reactivating chemoreceptor functioning (e.g. with an implantable electrical stimulator/blocker).

Carotid body modulation may include methods and systems for the thermal ablation of tissue via thermal heating mechanisms. Thermal ablation may be achieved due to a direct effect on tissues and structures that are induced by the thermal stress. Additionally or alternatively, the thermal disruption may at least in part be due to alteration of vascular or peri-vascular structures (e.g. arteries, arterioles, capillaries or veins), which perfuse the carotid body and neural fibers surrounding and innervating the carotid body (e.g. nerves that transmit afferent information from carotid body chemoreceptors to the brain). Additionally or alternatively thermal disruption may be due to a healing process, fibrosis, or scarring of tissue following thermal injury, particularly when prevention of regrowth and regeneration of active tissue is desired. As used herein, thermal mechanisms for ablation may include both thermal necrosis or thermal injury or damage (e.g., via sustained heating, convective heating or resistive heating or combination). Thermal heating mechanisms may include raising the temperature of target neural fibers above a desired threshold, for example, above a body temperature of about 37° C. e.g., to achieve thermal injury or damage, or above a temperature of about 45° C. (e.g. above about 60° C.) to achieve thermal necrosis. It is understood that both time of heating, rate of heating and sustained hot or cold temperature are factors in the resulting degree of injury.

In addition to raising temperature during thermal ablation, a length of exposure to thermal stimuli may be specified to affect an extent or degree of efficacy of the thermal ablation. For example, the length of exposure to thermal stimuli may be for example, longer than or equal to about 30 seconds, or even longer than or equal to about 2 minutes. Furthermore, the length of exposure can be less than or equal to about 10 minutes, though this should not be construed as the upper limit of the exposure period. A temperature threshold, or thermal dosage, may be determined as a function of the duration of exposure to thermal stimuli. Additionally or alternatively, the length of exposure may be determined as a function of the desired temperature threshold. These and other parameters may be specified or calculated to achieve and control desired thermal ablation.

In some embodiments, ablation of carotid body or carotid body nerves may be achieved via direct application of ablative energy to target tissue. For example, an ablation element may be applied at least proximate to the target, or an ablation element may be placed in a vicinity of a chemosensor (e.g. carotid body). In other embodiments, thermally-induced ablation may be achieved via indirect generation or application of thermal energy to the target neural fibers, such as through application of an electric field (e.g. radiofrequency, alternating current, and direct current), to the target neural fibers. For example, thermally induced ablation may be achieved via delivery of a pulsed or continuous thermal electric field to the target tissue such as RF and pulsed RF, the electric field being of sufficient magnitude or duration to thermally induce ablation of the target tissue (e.g., to heat or thermally ablate or cause necrosis of the targeted tissue). Additional and alternative methods and apparatuses may be utilized to achieve ablation, as described hereinafter.

### Transmural Ablation:

An endovascular catheter for transmural ablation may be designed and used to deliver an ablation element through a patient's vasculature to an internal surface of a vessel wall proximate a target ablation site. An ablation element may be, for example, a radiofrequency electrode or a virtual electrode. The ablation element may be made from radiopaque material or comprise a radiopaque marker and it may be visualized using fluoroscopy to confirm position. Alternatively, a contrast solution may be injected through a lumen in the ablation element to verify position. Ablation energy may be delivered, for example from a source external to the patient such as a generator or console, to the ablation element and through the vessel wall and other tissue to the target ablation site.

A temporary neural blockade may be applied to test a response to therapy prior to a more permanent ablative or disruptive ablation. For example application of cold can be used to temporarily block carotid body and carotid body nerves. Blockade of nerves that are desired to protect, rather than ablate, may lead to repositioning of the catheter. Such blockade can be noted by observing eyes of the patient, tongue, throat or facial muscles or by monitoring patient's heart rate and respiration. Following ablation, the catheter can be removed from the patient. An actuator in a handle may be used to deploy a deployable structure at a distal region of a catheter, which may be, for example: wires, resilient wires with soft tip, pinching prongs a deployable mesh, cage, basket, or helix that radially expands to secure the distal end of the catheter in the vessel and causes an ablation element to advance through a vessel wall. Alternatively a deployable structure may be an inflatable balloon that is deployed by injecting air or liquid (e.g. saline) into a hub in a proximal region of a catheter.

### Embodiments of Ionic Liquid Stream Catheters for Carotid Body Ablation

FIG. 1A is an illustration of a patient 1 in supine position depicting vascular access with the distal end 9 of vascular sheath 8 delivered to the region of a target carotid body 2, being a common carotid artery 3 through a femoral artery 6, abdominal aorta 5, and aortic arch 4 using a femoral artery puncture 7. Alternatively, the common carotid artery 3 may be accessed from a puncture in a brachial artery, radial artery or any other suitable artery. An endovascular procedure may involve the use of a guide wire, delivery sheath, guide catheter, introducer catheter, or introducer. Furthermore, these devices may be steerable and torqueable (i.e. able to conduct rotation from proximal to distal end).

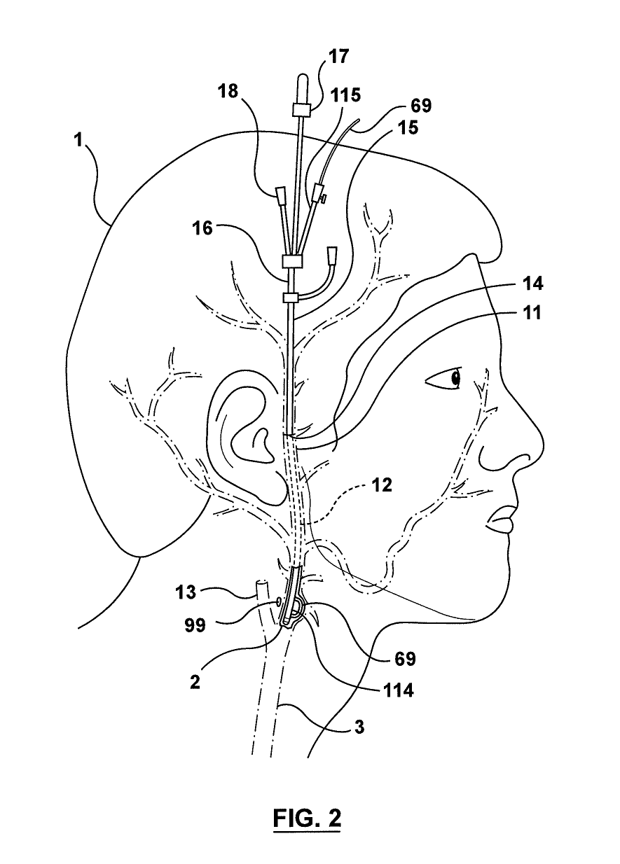

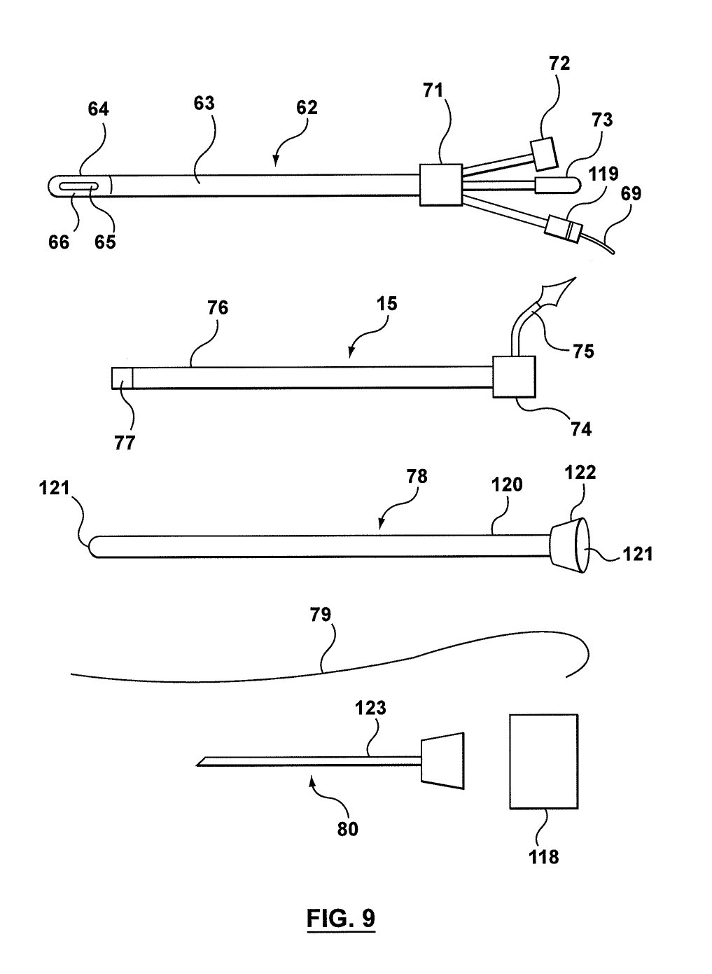

FIG. 2 is an illustration of the right side of the head of a patient 1 depicting vascular access to the right external carotid artery 12 using the right superficial temporal artery 11 for the purpose of carotid body ablation. As depicted, a carotid body ablation catheter 16 that utilizes an ionic liquid stream is shown in position for ablation of the right carotid body 99 located in the vicinity of the carotid bifurcation 2 between the internal carotid artery 13 and the external carotid artery 12, which are the two major branches of common carotid artery 3. Ablation element 114 is shown being pushed against the wall of external carotid artery 12 in the direction of carotid body 99 by push wire 69. Ablation catheter 16 is placed into external carotid artery 12 through introducer sheath assembly 15. Introducer sheath assembly 15 is inserted into the superficial temporal artery 11, through superficial temporal puncture 14 using a superficial temporal artery access kit depicted in FIG. 9 and described in detail below. As an alternative, the superficial temporal artery may be accessed using a surgical cut-down, which may, or may not utilized introducer sheath assembly 15. Carotid body ablation catheter 16 comprises a fluid channel between the vicinity of ablation element 114, and fluid connector 18, for injection of fluids, including ionic liquids, or contrast agents into the vicinity of carotid bifurcation 2 to facilitate radiological, or ultrasonic guidance for positioning ablation element 114 against the wall of external carotid artery 12 as shown. In addition to the use of contrast agents, ablation element 114 and/or push wire 22 may be configured to provide for an unambiguous identification of position within the vasculature under radiographic or ultrasonic imaging. Ablation catheter 16 may be configured to translate rotational forces from the proximal end of the catheter residing outside of patient 1 body to the region of ablation element 114 to facilitate radial positioning ablation element 114, which may comprise a knitted, coiled or woven structure within the shaft of ablation catheter 16. Ablation element 114 is connectable to a radiofrequency energy source, not shown, using ablation energy connector 17, and to source of pressurized ionic liquid, not shown using fluid connector 18. Also depicted is push wire port 115, which is configured to hold push wire 69 in its desired position. Superficial temporal arteries in adults typically range from approximately 2.25 mm in diameter to 3.25 mm in diameter, therefore, to ensure continued blood flow past superficial temporal artery puncture 14, and to avoid distal thrombosis, the caliber of introducer sheath assembly 15 or ablation catheter 16 may be smaller than superficial temporal artery 11 (e.g. about 2-3 mm). As depicted, carotid body ablation catheter 16 is a generic representation of a range of carotid body ablation catheter types that utilize ionic liquid streams. Ablation element 114 may be configured for mono-polar or bipolar radiofrequency energy ablation, or another ablation modality. As depicted, push wire 69 is used to push ablation element 114 against the wall of external carotid artery 12, however, there are alternative mechanisms that could be used, including using an internal pull wire to laterally deflect ablation element 114 against the wall of external carotid artery 12, or another mechanism may be used.

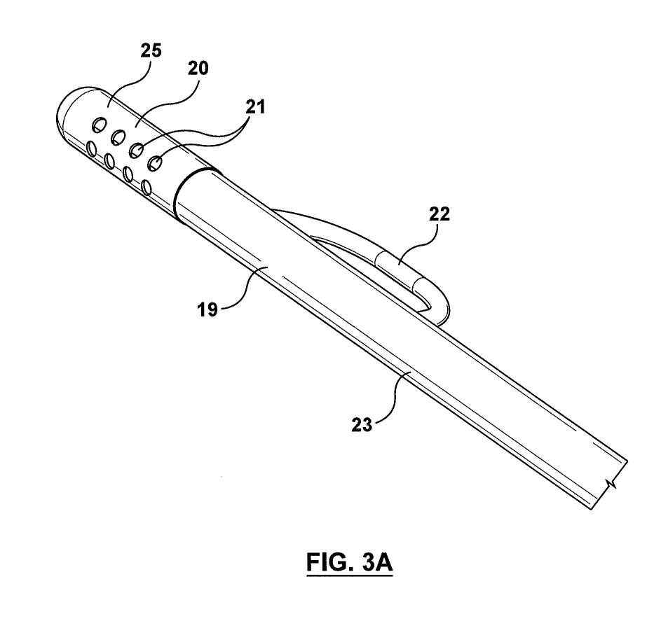

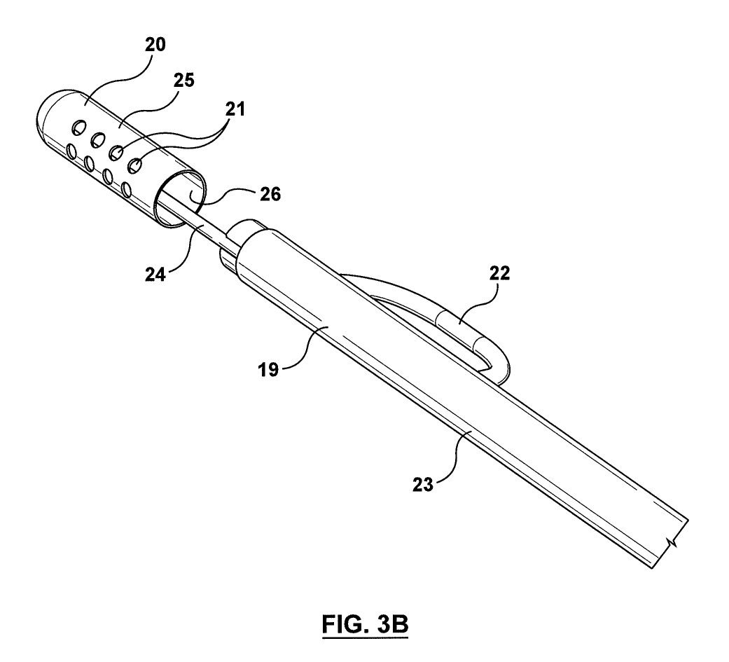

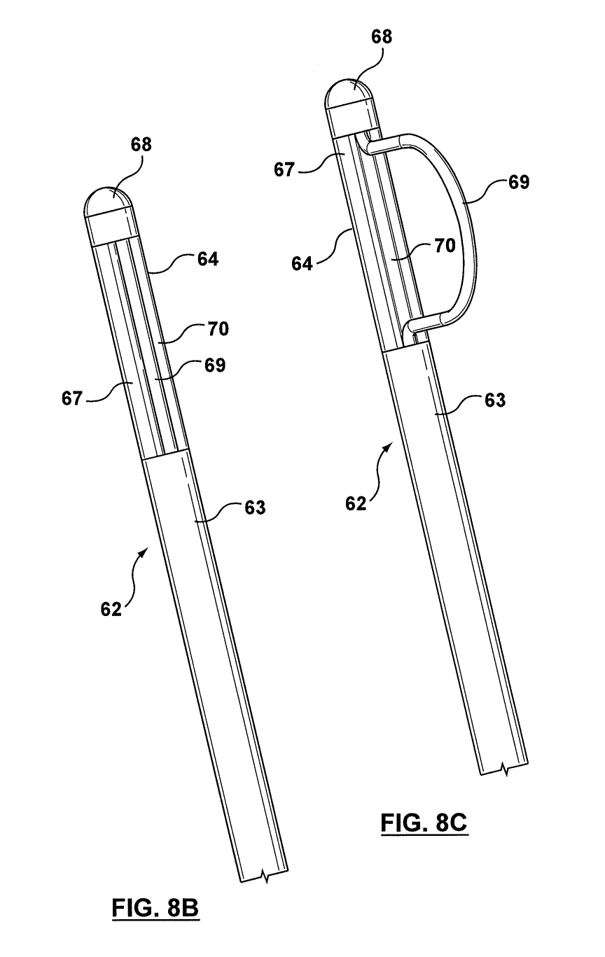

FIG. 3A is an illustration of the distal end of Ionic Liquid Electrode Carotid Body Ablation (ILE-CBA) catheter 19 that utilizes an RF tissue contact electrode comprising a stream of ionic liquid. FIG. 3B is an illustration of ILE-CBA catheter 19 in exploded view. ILE-CBA catheter 19 comprises catheter shaft 23, electrode cap 20, electrical conductor 24, push wire 22, and proximal terminal, not shown. Catheter shaft 23 is approximately 100 cm to 120 cm long and between approximately 6 French to 9 French in caliber, when configured for use through a femoral artery puncture, as illustrated in FIG. 1A and described in detail above, or may be shorter if an alternative vascular access point is used, such as a radial, brachial or subclavian artery puncture. Catheter shaft 23 comprises a central lumen, which provides fluidic communication between the interior of electrode cap 20 and a fluid connector associated with the proximal terminal, not shown. Catheter shaft 23, may also comprise a lumen configured to house electrical conductor 24, and an additional lumen to house push wire 22. Catheter shaft 23 may be formed by extrusion of a polymeric compound commonly used in catheter making such as polyurethane, polyethylene, nylon, PEBAX®, etc. Catheter shaft 23 may also comprise a woven, knitted or coiled structure within its walls to provide torsional rigidity, while maintaining catheter shaft flexibility, which provides for high fidelity radial positioning of electrode cap 20 within an external carotid artery. Electrode cap 20 comprises a hollow cylindrical structure, which is open at its proximal end, and closed with a substantially hemispherical distal bulkhead, as shown. Electrode cap 20 comprises a substantially electrically conductive inner surface 26, and a substantially electrically non-conductive outer surface 25. Alternatively, an electrode and a cap may be distinct parts wherein the electrode may be a conductive material contained within the cap and the cap may be a non-conductive material. Electrode cap 20 comprises at least one lateral fenestration(s) 21 configured for fluidic communication between the interior and exterior of electrode cap 20. The cross sectional area of lateral fenestration(s) 21 defines the maximum current density within the radiofrequency ablation circuit, and may be manipulated for specific desired ablation lesion morphology. For a given radiofrequency current and ionic liquid flow rate, a smaller cross sectional area will result in higher tissue ablation temperatures, and a more focused lesion, where high cross sectional fenestration are will result in lower ablation tissue temperatures and a larger and more diffuse ablation lesions. The cross sectional area of fenestration(s) 21 may be between approximately 0.2 square millimeters to 6 square millimeters. Electrode cap 20 outside diameter may approximate the outer diameter of catheter shaft 23, and has a wall thickness between approximately 0.005 inches and 0.025 inches. Electrode cap 20 may be formed from a metallic tube such as stainless steel, or a precious, and more radiopaque metal alloy such as a gold or platinum alloy, and insulated surface 25 comprising a polymeric or ceramic electrically insulating coating may then be applied to the metal substrate, thereby forming electrode cap 20. Electrical conductor 24 is connected (e.g. welded or soldered) to the inner surface 26 of electrode cap 20, at its distal end, and connected to an electrical connector, not shown associated with the proximal terminal configured for connection to one pole of a radiofrequency energy generator, not shown. Electrode cap 20 is mounted on the distal end of catheter shaft 23 with lateral fenestration(s) 21 radially positioned in diametric opposition to push wire 22, as shown. Push wire design and operation is illustrated in FIG. 8A, and FIG. 8B, and described in detail below. Proximal terminal, not shown, comprises a fluid connector in communication with the central lumen of catheter shaft 23, and is configured for connecting the central lumen of catheter shaft 23, and interior space of electrode cap 20 with a pressurized source of ionic liquid. Proximal terminal further comprises an electrical connector as previously described, and a push wire actuation mechanism, which will be described in detail below.

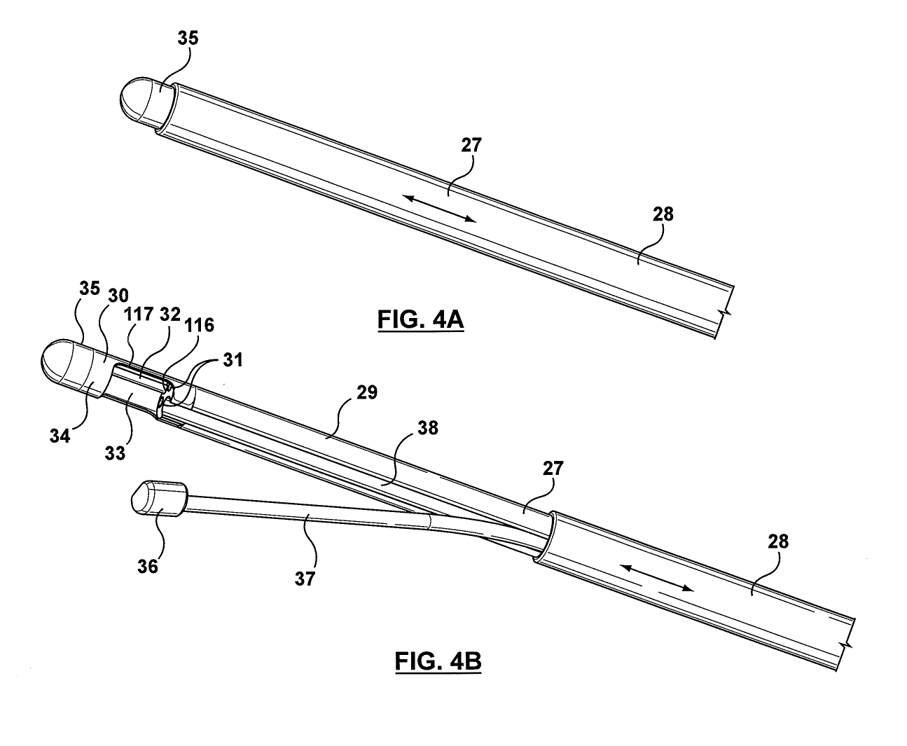

FIG. 4A is an illustration of the distal end of an Ionic Liquid Electrode Carotid Body Ablation Forceps (ILE-CBA-F) catheter 27 that utilizes a radiofrequency tissue contact electrode comprising a stream of ionic liquid in its insertion and navigation configuration. FIG. 4B is an illustration of the ILE-CBA-F catheter 27 with the forceps deployed. ILE-CBA-F catheter 27 comprises outer sheath 28, inner shaft 29, electrode hood, 30, electrical conductor 32, distal tip 35, forceps jaw 36, forceps arm 37, and proximal terminal, not shown. Inner shaft 29 comprises at least one fluid lumen(s) 31, wire lumen 116, and forceps arm channel 38. Electrode hood 30 comprises inner electrically conductive surface 33, outer electrically insulated surface 34, and fenestration 117. Proximal terminal comprises an electrical connector connected to electrical conductor 32 and configured for connection to one pole of a radiofrequency energy generator, not shown, a fluid connector in fluidic communication with fluid lumen(s) 31 configured for connection to a pressurized source of ionic liquid, and an actuator configured for user positioning of outer sheath 28. Forceps arm 37 is configured to reside within forceps arm channel 38 of inner shaft 29, when outer sheath 28 is in its distal position as shown if FIG. 4A, and further configured with a spring bias that provides lateral displacement as outer sheath 28 is withdrawn in the proximal direction as shown in FIG. 4B. Forceps arm 37 may be formed out of a hypodermic tube, or a wire made of a super elastic alloy such as Nitinol®, or another alloy such as stainless steel. The cross sectional shape of forceps arm may other than round (i.e., rectangular, D shaped, etc.) to provide transverse rigidity while maintaining lateral flexibility. Forceps arm 37 may terminate just proximal to the end of outer sheath 28 in its proximal position as shown in FIG. 4B, or may extend substantially the entire length of ILE-CBA-F catheter. Forceps jaw 36 is configured to reside within electrode hood 30 when outer sheath 28 is in its distal position shown in FIG. 4A, and be positioned in lateral opposition to fenestration 117 of electrode hood 30 in its deployed position as shown in FIG. 4B. The opposing distance between forceps jaw 36 and fenestration 117 may be adjusted by positioning outer sheath in a distal or proximal direction in between the positions shown in FIGS. 4A and 4B. Inner catheter shaft 29 may have a caliber between approximately 6 French and 9 French, and outer sheath is configured in a sliding relationship with inner catheter shaft 29, as shown, and has a wall thickness between 0.010, and 0.025 inches. The overall working length of ILE-CBA-F catheter 27 is between approximately 100 cm and 120, when configured for access to the vicinity of a carotid body by means of a femoral artery puncture as illustrated in FIG. 1, and described in detail above, or may be shorter if it is configured for an alternative insertion site such as a brachial or sub-clavicle artery. Inner catheter shaft 29 may be formed by extrusion of a polymeric compound commonly used in catheter making such as polyurethane, polyethylene, nylon, PEBAX®, etc. Inner catheter shaft 29 may also comprise a woven, knitted or coiled structure within its walls to provide torsional rigidity, while maintaining catheter shaft flexibility, which provides for high fidelity radial positioning of electrode cap 30 within an external or internal carotid artery. Outer sheath 28 may also be formed by extrusion of a polymeric compound commonly used in catheter making such as polyurethane, polyethylene, nylon, PEBAX®, etc. Outer sheath may also comprise a woven, knitted or coiled structure within its walls to provide column strength for manipulating forceps arm 37, while maintaining catheter flexibility. Electrode cap 30 may be machined from metallic tubing with outer diameter approximating the caliber of inner catheter shaft 29, in the figuration shown in FIG. 4B. An electrically insulating polymer coating or ceramic coating may then be applied to the external surfaces of electrode hood 30 forming electrically insulating surface 34. Electrical wire 32 is attached to electrically conductive inner surface 33, which may be a welded or soldered connection, thereby making electrically conductive surface 33 connectable to one pole of a radiofrequency energy generator by means of electrical conductor wire 32, and electrical connector of the proximal terminal, not shown. Distal tip 35 may be configured with a hemispherical distal surface as shown, and may be formed by a machining of molding process. Distal tip 35 may be formed a metal chosen for its radiopacity properties to assist in radiographic surgical guidance. Distal tip 35 is attached to the distal end of electrode hood 30, by means appropriate by those skilled in the art of catheter design and assembly. In an alternate embodiment, forceps jaw 36 may be configured for electrical connection to the second pole of a radiofrequency energy generator, thereby forming a bi-polar electrode pair between inner electrically conductive surface 33 of electrode hood 30, and forceps jaw 36.

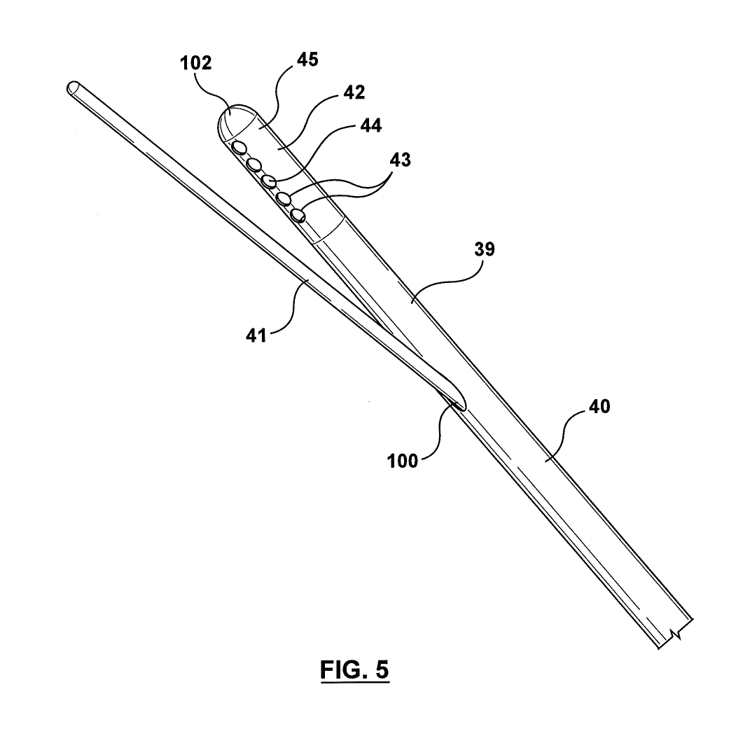

FIG. 5 is an illustration of the distal end of an Ionic Liquid Electrode Carotid Body Ablation, with a Side-Exiting Guidewire (ILE-CBA-SG) catheter 39 configured for use in a carotid bifurcation that utilizes a radiofrequency tissue contact electrode comprising a stream of ionic liquid. ILE-CBA-SG catheter 39 comprises catheter shaft 40, electrode hood 42, and proximal terminal, not shown. Catheter shaft 40 has a caliber between approximately 5 French and 8 French, and a working length between approximately 100 cm and 120 cm when configured for use through a femoral artery puncture as illustrated in FIG. 1, and described in detail above, or may be shorter when configured for use through an alternate arterial puncture site such as a brachial artery or sub-clavian artery puncture site. Catheter shaft 40 comprises a central lumen, which provides fluidic communication between the interior of electrode cap 42 and a fluid connector associated with the proximal terminal, not shown. Catheter shaft 40, also comprises guidewire lumen terminating at lateral guidewire port 100 as shown, and may also comprise a lumen configured to house an electrical conductor, not shown. Catheter shaft 40 may be formed by extrusion of a polymeric compound commonly used in catheter making such as polyurethane, polyethylene, nylon, PEBAX®, etc. Catheter shaft 40 may also comprise a woven, knitted or coiled structure within its walls to provide torsional rigidity, while maintaining catheter shaft flexibility, which provides for high fidelity radial positioning of electrode cap 42 within a carotid artery. Electrode cap 42 comprises a hollow cylindrical structure, which is open at its proximal end, and closed with a substantially hemispherical distal bulkhead, as shown. Electrode cap 42 comprises a substantially electrically conductive inner surface 44, and a substantially electrically non-conductive outer surface 45. Electrode cap 42 comprises at least one lateral fenestration(s) 43 configured for fluidic communication between the interior and exterior of electrode cap 42. The cross sectional area of lateral fenestration(s) 43 defines the maximum current density within the radiofrequency ablation circuit, and may be manipulated for specific desired ablation lesion morphology. For a given radiofrequency current and ionic liquid flow rate, a smaller cross sectional area will result in higher tissue ablation temperatures, and a more focused lesion, where high cross sectional fenestration area will result in lower ablation tissue temperatures and a larger and more diffuse ablation lesion. The cross sectional area of fenestration(s) 43 may be between approximately 0.2 square millimeters to 6 square millimeters. Electrode cap 42 outside diameter may approximate the outer diameter of catheter shaft 23, and has a wall thickness between approximately 0.005 inches and 0.025 inches. Electrode cap 42 may be formed from a metallic tube such as stainless steel, or a precious, and more radiopaque metal alloy such as a gold or platinum alloy, where insulated surface 45 comprising a polymeric or ceramic electrically insulating coating may then be applied to the metal substrate, thereby forming electrode cap 42. The electrically conductive interior surface 44 of electrode cap 42 is connected to an electrical connector associated with the proximal terminal by an electrical wire, not shown residing within catheter shaft 40, providing a means for connecting electrically conductive inner surface 44 to one pole of a radiofrequency energy generator. Proximal terminal also comprises a fluid connector in fluid communication with the central lumen, not shown, of catheter shaft 40 configured for connection to a pressurized source of ionic liquid. Electrode cap 42 is bonded to the distal end of catheter shaft 40 with fenestration(s) 43 in radial or lateral alignment with lateral guidewire port 100 as shown.

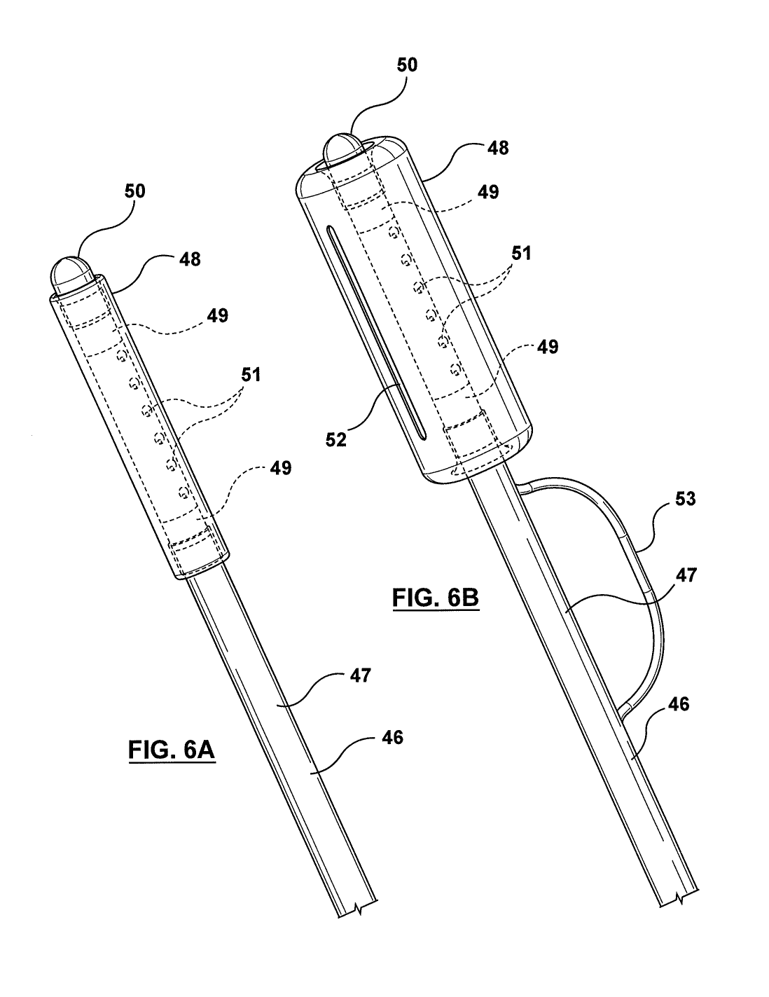

FIG. 6A is an illustration of the distal end of an Ionic Liquid Electrode Carotid Body Ablation Balloon (ILE-CBA-B) catheter 46 that utilizes an RF tissue contact electrode comprising a stream of ionic liquid showing the balloon 48 deflated. FIG. 6B is an illustration of the distal end of an ILE-CBA-B catheter 46 showing balloon 48 inflated. ILE-CBA-B catheter 46 comprises catheter shaft 47, balloon 48, radiofrequency electrodes 49 that may also be radiopaque markers, push wire 53, distal tip 50, and proximal terminal, not shown. Catheter shaft 47 is approximately 10 cm to 20 cm long and between approximately 3 French to 6 French in caliber, when configured for use through a temporal artery puncture, as illustrated in FIG. 2 and described in detail above, or may be longer (e.g. between about 100 to 120 cm, about 110 cm), and of larger caliber (e.g. 3 to 6 French) if an alternative vascular access point is used, such as a femoral, radial, brachial, or subclavian artery puncture. Catheter shaft 47 comprises a central lumen, which provides fluidic communication between the interior of balloon 48 and a fluid connector associated with the proximal terminal, not shown. Catheter shaft 47, may also comprise a lumen configured to house an electrical conductor, not shown that provides electrical communication between one or more radiofrequency electrode/radiopaque marker(s) 49 and an electrical connector associated with the proximal terminal configured for electrical connection to one pole of a radiofrequency energy generator, not shown. Catheter shaft 47 also comprises a lumen, not shown to house push wire 53. Catheter shaft 47 may be formed by extrusion of a polymeric compound commonly used in catheter making such as polyurethane, polyethylene, nylon, PEBAX®, etc. Catheter shaft 47 may also comprise a woven, knitted or coiled structure within its walls to provide torsional rigidity, while maintaining catheter shaft flexibility, which provides for high fidelity radial positioning of the distal end of ILE-CBA-B catheter 46 within an external carotid artery. Catheter shaft 47 has at least one fluid port(s) 51 in fluidic communication between its central lumen, not shown, and the interior of balloon 48. Balloon 48 comprises longitudinal fenestration 52, which provides fluidic communication between the interior and exterior of balloon 48. Balloon 48 may comprise a compliant elastomeric structure formed from a silicone rubber compound, a urethane rubber compound, or a latex rubber compound. Alternatively, balloon 48 may be formed from a substantially non-compliant polymer, such as PET, or polyethylene. Those skilled in the art of balloon catheter design and construction are familiar means for forming and assembling balloons as described here within, therefore, no further explanation is warranted. Longitudinal fenestration 52, is configured to communicate pressurized ionic liquid from the interior of balloon 48 into the arterial space surrounding balloon 48, with a resistance to the flow of ionic liquid sufficient to cause a pressure within the interior of balloon 48 sufficient to substantially inflate balloon 48. Longitudinal fenestration 52 may be formed within the wall of balloon 48 by incising the wall, without removing wall material, alternatively, longitudinal fenestration 52 may be machined into the wall of balloon 48, which may involve a laser machining operation. The cross sectional area of longitudinal fenestration 52 defines the maximum current density within the radiofrequency ablation circuit, and may be manipulated for specific desired ablation lesion morphology. For a given radiofrequency current and ionic liquid flow rate, a smaller cross sectional area will result in higher tissue ablation temperatures, and a more focused lesion, where high cross sectional fenestration area will result in lower ablation tissue temperatures and a larger and more diffuse ablation lesion. The cross sectional area of fenestration(s) 43 may be between approximately 0.2 square millimeters to 2.0 square millimeters. The cross sectional area of longitudinal fenestration 52 may be adjusted by adjusting the flow rate of ionic liquid through fenestration 52. As the flow rate of ionic liquid is increased, the pressure within balloon 48 increases, causing the surface area of balloon 48, and cross sectional area of longitudinal fenestration 52 to increase. Balloon 48 is mounted on catheter shaft 47 with longitudinal fenestration 52 positioned in diametric opposition to push wire 53. FIGS. 8A and 8B provide a more detailed illustration of push-wire design and function, along with a detailed description below.

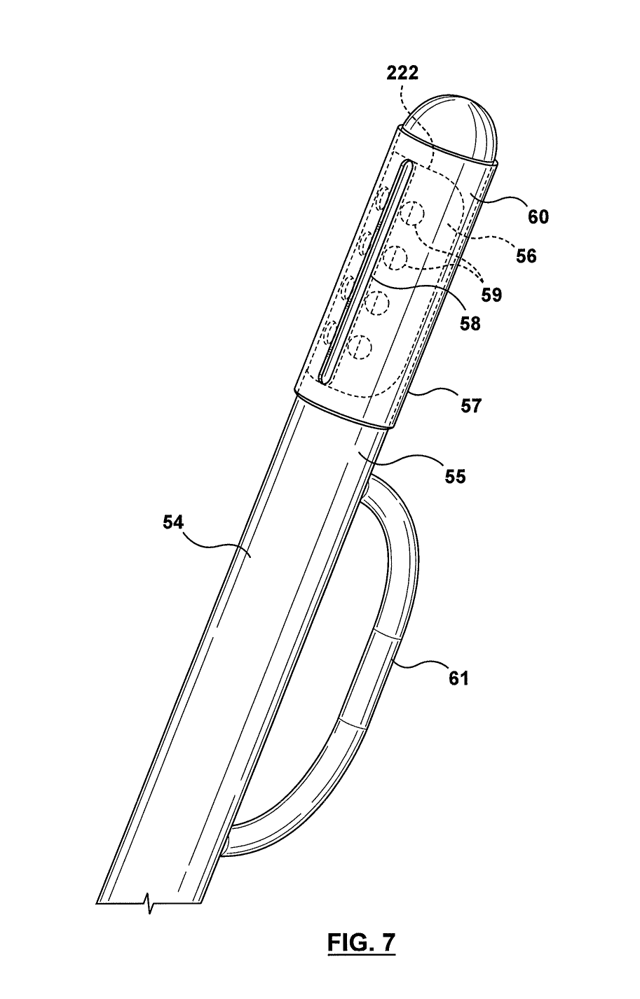

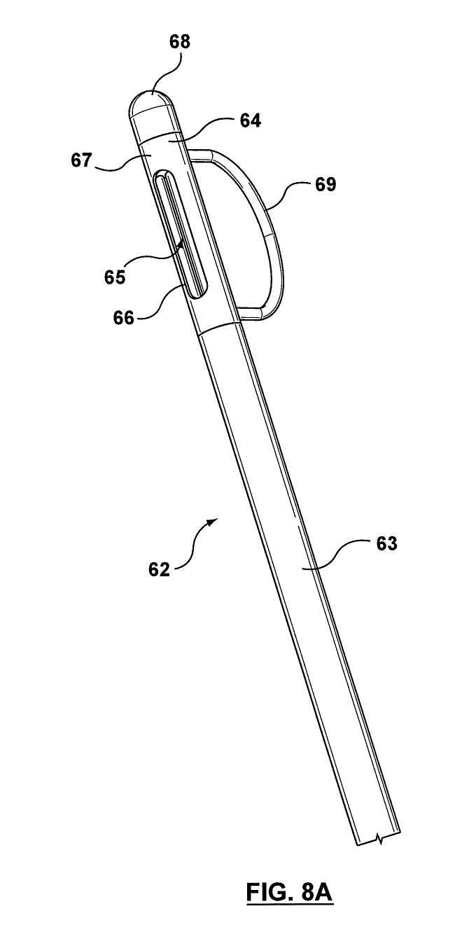

FIG. 7 is an illustration of the distal end of an Ionic Liquid Electrode Carotid Body Ablation Elastic Slit (ILE-CBA-ES) catheter 54 that utilizes an RF tissue contact electrode comprising a stream of ionic contrast liquid where the liquid flows through a slit 58 in an elastomeric membrane 57. ILE-CBA-ES catheter 54 comprises catheter shaft 55, electrode cap 56, elastic membrane 57, comprising slit 58, push wire 61, and proximal terminal, not shown. ILE-CBA-ES catheter 54 is similar to ILE-CBA catheter 19 depicted in FIGS. 3A and 3B, and described in detail above except for the addition of elastic membrane 57, therefore, further description of ILE-CBA-ES catheter 54 will be limited to the addition of membrane 57. Elastic membrane 57, comprises an elastomeric tube, which may be formed from a silicone rubber, urethane rubber, latex rubber, or other type of rubber. The inner diameter of elastic membrane tube 57 is between approximately 40% and 80% of the outer diameter of electrode cap 56 in its relaxed state. Slit 58, may be formed by incision, where no material is removed from elastic membrane 57, or may be formed by a machining operation, which may comprise a laser machining operation. Elastic membrane 57 is bonded to electrode cap 56, with adhesive 60, with slit 58 positioned between fenestrations 59, as shown. Adhesive 60 is masked to avoid adhesion of membrane 57 to electrode cap 56 in the vicinity of fenestrations 59, as represented by the dotted line 222. Since membrane tube 57, in its relaxed state is smaller in diameter than electrode cap 56, fenestrations 59 are firmly covered by membrane 57 creating a liquid and gas tight seal. When, pressurized ionic liquid is introduced into the central lumen of ILE-CBA-ES catheter 54, the pressure of the ionic liquid overcomes the closing pressure of elastic membrane 57 causing the ionic liquid to flow out fenestrations 59 and through slit 58 to create a liquid electrode at the outlet of slit 58. In addition, elastic membrane 57 and slit 58 form a one way fluid valve that may be useful for purging air from ILE-CBA-ES catheter 54 prior to insertion in a patient's blood stream.

FIG. 8A is an isometric front view illustration of the distal end of an Ionic Liquid Electrode Carotid Body Ablation Small Caliber (ILE-CBA-SC) catheter 62 configured for use by trans-superficial temporal artery access to the region of a target carotid body and transmural ablation from within an external carotid artery. FIG. 8B is an isometric rear view illustration of ILE-CBA-SC catheter 62 push wire 69 retracted. FIG. 8C is a rear view illustration of ILE-CBA-SC catheter 62 showing push wire 69 extended. ILE-CBA-SC catheter 62 comprises catheter shaft 63, electrode cap 64, distal tip 68, push wire 69, and proximal terminal, not shown. Catheter shaft 63 working length is approximately 10 cm to 25 cm long and between approximately 2 to 3 French in caliber, when configured for use through a superficial temporal artery puncture, as illustrated in FIG. 2 and described in detail above, or may be longer (e.g., between about 100 to 120 cm, about 110 cm) and have a larger caliber (e.g., about 3 to 6 French) if an alternative vascular access point is used, such as a radial, brachial, or femoral artery puncture. Catheter shaft 63 comprises a central lumen, which provides fluidic communication between the interior of electrode cap 64 and a fluid connector associated with the proximal terminal, not shown. Catheter shaft 63, may also comprise a lumen configured to an house electrical conductor, not shown, and an additional lumen to house push wire 69. Catheter shaft 63 may be formed by extrusion of a polymeric compound commonly used in catheter making such as polyurethane, polyethylene, nylon, PEBAX®, etc. Catheter shaft 63 may also comprise a woven, knitted or coiled structure within its walls to provide torsional rigidity, while maintaining catheter shaft flexibility, which provides for high fidelity radial positioning of electrode cap 64 within an external carotid artery. Electrode cap 64 comprises a hollow cylindrical structure, which is open at its proximal end, and closed with a substantially hemispherical distal bulkhead 68, as shown. Electrode cap 64 comprises a substantially electrically conductive inner surface 65, and a substantially electrically non-conductive outer surface 67. Electrode cap 64 comprises lateral fenestration 66 configured for fluidic communication between the interior and exterior of electrode cap 64. The cross sectional area of lateral fenestration 66 defines the maximum current density within the radiofrequency ablation circuit, and may be manipulated for specific desired ablation lesion morphology. For a given radiofrequency current and ionic liquid flow rate, a smaller cross sectional area will result in higher tissue ablation temperatures, and a more focused lesion, where high cross sectional fenestration are will result in lower ablation tissue temperatures and a larger and more diffuse ablation lesion. The cross sectional area of fenestration 66 may be between approximately 0.2 square millimeters to 6 square millimeters. Electrode cap 64 outside diameter may approximate the outer diameter of catheter shaft 63, and has a wall thickness between approximately 0.005 inches and 0.025 inches. Electrode cap 64 may be formed from a metallic tube such as stainless steel, or a precious, and more radiopaque metal alloy such as a gold or platinum alloy, and insulated surface 67 comprising a polymeric or ceramic electrically insulating coating may then be applied to the metal substrate forming electrode cap 64. The electrical conductor, not shown, is connected (e.g., welded or soldered) to the inner surface 65 of electrode cap 64, at its distal end, and connected to an electrical connector, not shown associated with the proximal terminal configured for connection to one pole of a radiofrequency energy generator, not shown. Electrode cap 64 is mounted on the distal end of catheter shaft 63 with lateral fenestration 66 radially positioned in diametric opposition to push wire 69, as shown. As best illustrated in FIGS. 8B and 8C, push wire 69 is anchored in the vicinity of distal end 68, and traverses push wire channel 70, and a dedicated lumen in catheter shaft 63, and exits through a Tuohy-Borst connector associated with proximal terminal, not shown. During insertion, push wire 69 is pulled with slight tension, and locked in place by the Tuohy-Borst connector, which results in push wire 69 residing within push wire channel 70 as shown in FIG. 8B. Once, the working end of ILE-CBA-SC catheter 62 adjacent to the target carotid body, catheter shaft 63 is rotated so that fenestration 66 is facing the target carotid body. Tuohy-Borst connector is loosened and the proximal end of push wire 69 is advanced in the distal direction, causing push wire 69 to buckle at the distal end as shown in FIG. 8C, and gently push fenestration 66 against the wall of the external carotid artery proximate to the target carotid body. The Tuohy-Borst connector may then be tightened to lock the push wire in its extended position during carotid body ablation. Alternatively, a push wire may be connected to an actuator associated with a proximal terminal of the catheter 62 (not shown) that extends and retracts the push wire a desired amount to deploy the push wire from the channel 70.

FIG. 9 is an illustration of a procedure kit for trans-temporal artery ablation of a carotid body, comprising: needle 80, guidewire 79, arterial introducer sheath 15, obturator 78, ILE-CBA-SC catheter 63, and instructions for use 118. Fluid connector 72 is in fluidic communication with the interior of electrode cap 64, and may be used to inject an imaging contrast agent to aid in positioning fenestration 66 for carotid body ablation, in addition to supplying an ionic liquid under pressure to electrode cap 64 during ablation. Radiofrequency energy connector 73 is in electrical communication with interior electrically conductive surface 65 of electrode cap 64 and is used to connect electrically conductive surface 65 to a pole of a radiofrequency energy generator. Tuohy-Borst connector 119, is configured to lock, push wire 69, in its desired operational position as previously described, while also providing a fluid tight seal around push wire 69. Alternatively, as previously described, a push wire may be advanced and retracted by an actuator associated with the proximal end of catheter 63. Introducer sheath 15 comprises sheath tube 76 comprising a thin walled hollow structure, introducer valve 74, fluid connector 75, and radiopaque marker 77. Sheath tube 76 has a working length of approximately 5 cm to 23 cm, with the working length being the distance from the distal end, to introducer valve 74. Sheath tube 76 is configured for a specific caliber carotid body ablation catheter where the inner diameter of sheath tube 76 is a fraction of a millimeter larger than the outside diameter of the corresponding ablation catheter. Sheath tube 76 has a wall thickness between approximately 0.25 mm and 0.75 mm, and is an extrusion of a flexible polymeric material which may be a polyurethane, polyethylene or other polymeric compound typically used in vascular catheter and sheath construction. Radiopaque marker 77 is bonded to sheath tube 76 in the vicinity of its distal tip and comprises a thin walled ring of radiopaque metal, or a paint comprising a radiopaque metal. Introducer valve 74 comprises an elastomeric valve configured to prevent blood from exiting the sheath when inserted into a superficial temporal artery, with, or without the ILE-CBA-SC catheter 63 inserted into introducer sheath 15. Those skilled in the art of introducer sheath construction are familiar with introducer valve design and construction, therefore, no further description is warranted. Fluid connector 75 is in fluidic communication with the inner lumen of introducer tube 76, and is used to insert and remove fluid from sheath tube 76. Obturator 78 is configured to facilitate insertion of introducer sheath 15 into a superficial temporal artery. Obturator 78 comprises obturator shaft 120, central guidewire lumen 121, and guidewire valve 122. Obturator shaft 120 is configured with an outer diameter approximately the same as the corresponding ILE-CBA-SC catheter 63, and has a working length approximately 0.5 cm to 2 cm longer than the working length of the corresponding introducer sheath 15. Obturator shaft 120 has a bullet shape formed on the distal end, and guidewire valve 122 mounted in the vicinity of the proximal end. Guidewire lumen a 121 is sized to accommodate a guidewire between approximately 0.014″ to 0.038″ and traverses the entire length of obturator shaft 120. Guidewire valve 122 is sized to accommodate the same size guidewire as guidewire lumen 121. Guidewire valve 122 is configured to prevent blood from exiting through guidewire lumen 121 during introducer sheath 5 insertion into a superficial temporal artery. Those skilled in the art of obturator construction are familiar with guidewire valve design and construction, therefore, no further description is warranted. Guidewire 79 is between approximately 0.014″ and 0.038″ and corresponds to the size of guidewire lumen 121 on obturator 78. Guidewire 79 has a length of approximately 20 cm to 50 cm, and may be uniform stiffness, or may have a distal end that is relatively floppy. Those skilled in the art of guidewire construction are familiar with guidewire design and construction, therefore, no further description is warranted. Puncture needle 80 comprises a hypodermic needle shaft 123, and needle hub 124. Hypotube shaft 123 has an inner diameter that is slightly larger than corresponding guidewire 79, which allows guidewire 79 to slide freely within hypodermic needle shaft 123. Needle shaft 123 has a sharpened distal tip 125 configured for puncture of the skin and insertion into a superficial temporal artery. Needle hub 124 is a female luer fitting configured for attachment of a syringe or Tuohy-Borst connector. Those skilled in the art of puncture needle construction are familiar with puncture needle design and construction, therefore, no further description is warranted. Directions-for-use 118 may comprise directions for: palpating a superficial temporal artery, puncturing the skin and inserting puncture needle 80 into the superficial temporal artery, inserting guidewire 79 through needle 80; removing needle 80 from the superficial temporal artery while leaving guidewire 79 in place; inserting obturator 78 into introducer sheath 15; sliding introducer sheath 15 and obturator into the superficial temporal artery over guidewire 79; removing obturator 78 while leaving introducer sheath 15 in place; inserting ILE-CBA-SC catheter 63 into the superficial temporal artery through introducer sheath 15; positioning fenestration 66 adjacent to a carotid body and pressing fenestration 66 against the wall of an external carotid artery using push wire 69 and Tuohy-Borst connector 119; connecting electrical connector 73 to one pole of a radiofrequency generator, not shown; placing an indifferent electrode, not shown, on the skin of the patient, and connecting the indifferent electrode to the second pole of the radiofrequency energy generator; connecting fluid connector 72 to a source of pressurized ionic liquid, not shown; selecting ablation energy parameters; initiating ionic liquid flow; activating and deactivating the radiofrequency generator; terminating ionic liquid flow; assessing ablation effectiveness; and further provide direction based determination of ablation effectiveness. Directions-for-use 118 may further describe patients who are indicated for carotid body ablation via superficial temporal artery puncture, patients who are contra-indicated for carotid body ablation via superficial temporal artery puncture, complications, which could be expected, and warnings of potential adverse events.

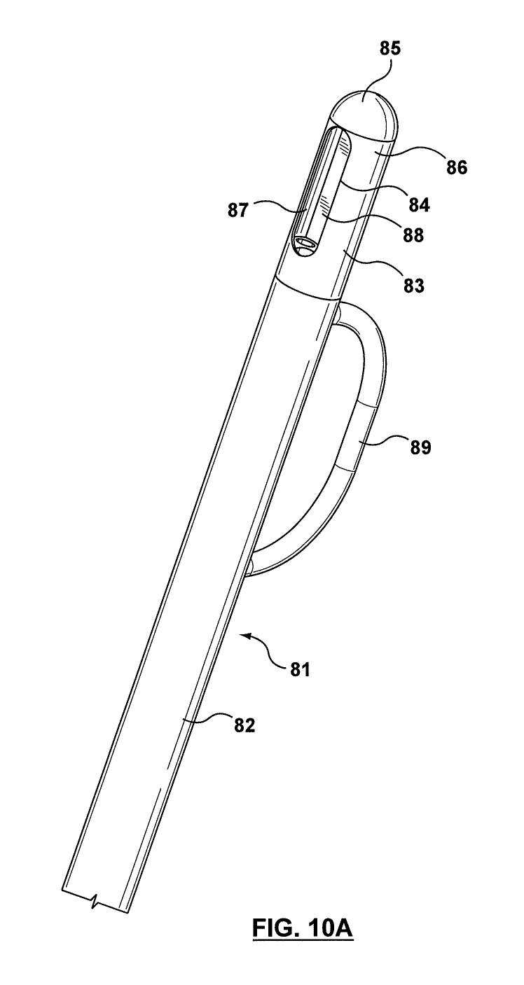

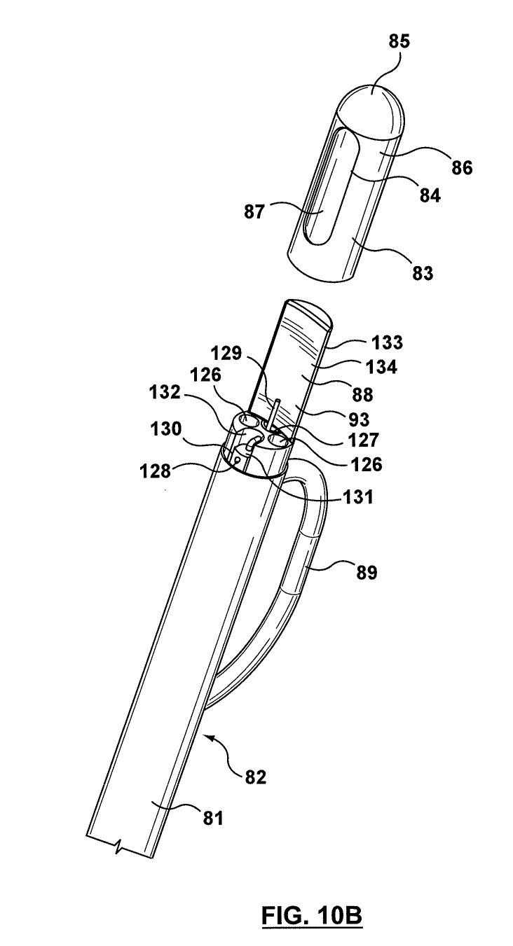

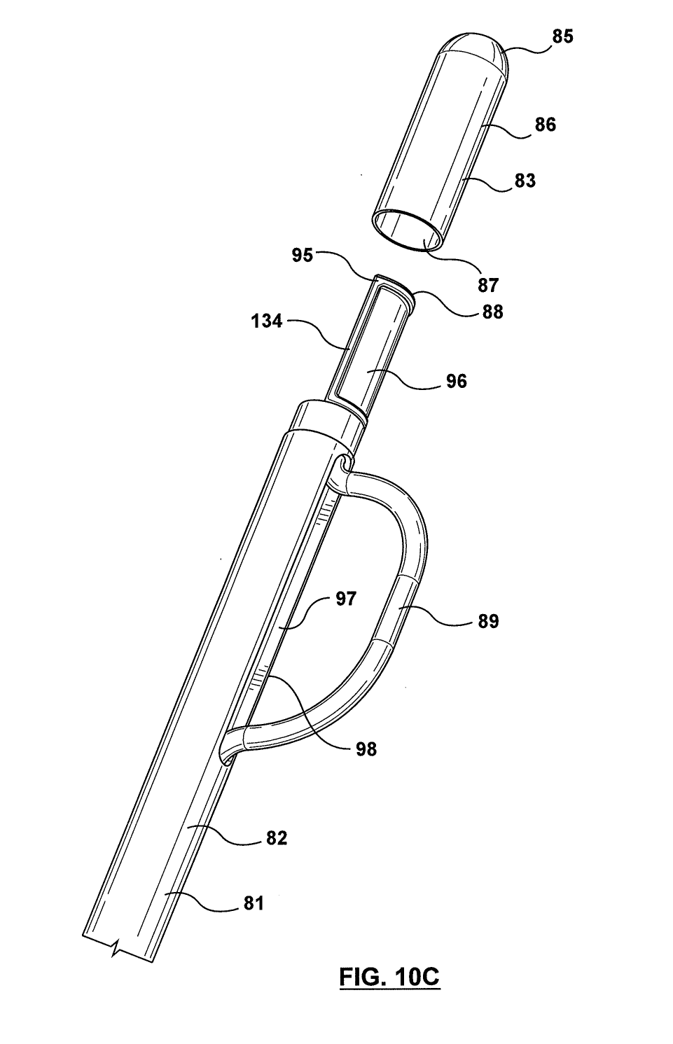

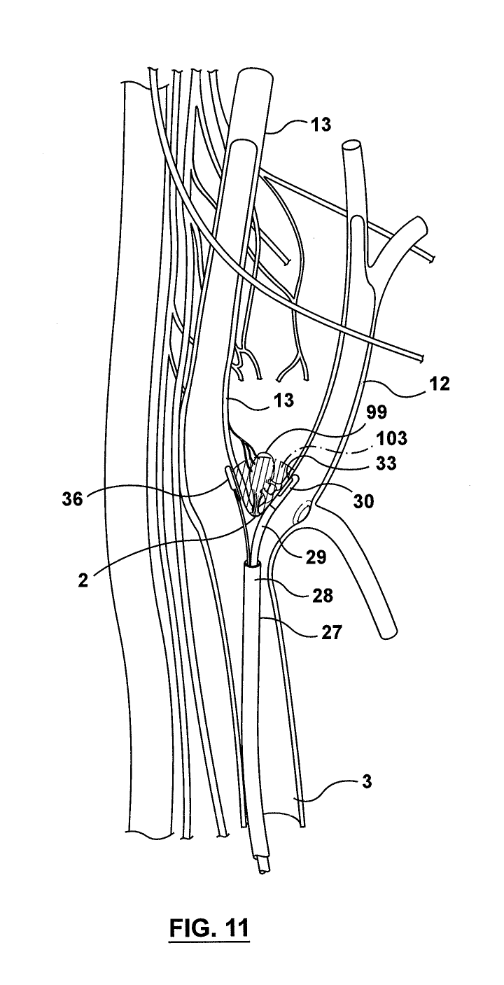

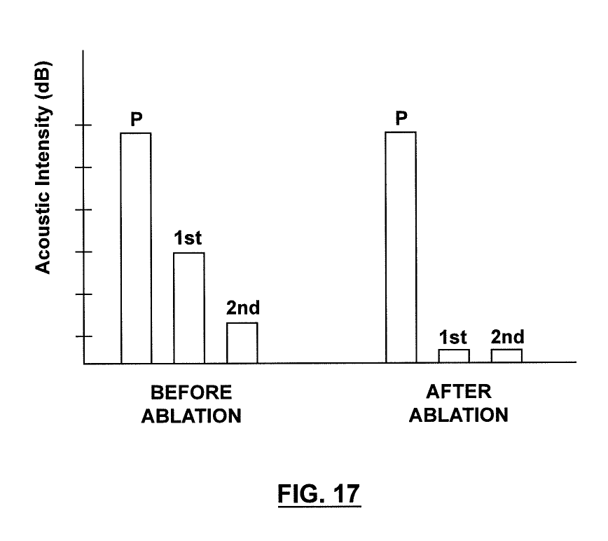

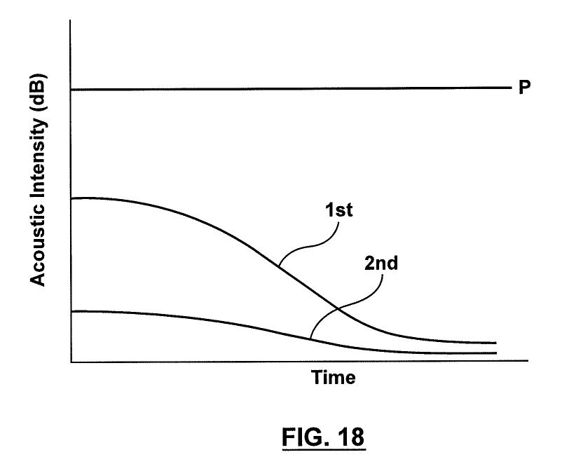

FIG. 10A is an illustration of the distal end of Ionic Liquid Electrode Carotid Body Ablation Dual Mode (ILE-CBA-DM) catheter 81 comprising radiofrequency, and ultrasonic energy modalities configured for use in conjunction with a systemically administered ultrasonic contrast agent that utilizes a stream of ionic liquid. FIG. 10B is an exploded view illustration of the front distal end of ILE-CBA-DM catheter 81. FIG. 10C is an exploded view illustration of the rear distal end of ILE-CBA-DM catheter 81. ILE-CBA-DM catheter 81 comprises catheter shaft 82, electrode hood 83, ultrasonic transducer 88, push wire 89, coaxial electrical cable 131, and proximal terminal, not shown. Catheter shaft 82 is approximately 100 cm to 120 cm long and between approximately 6 French to 9 French in caliber, when configured for use through a femoral artery puncture, as illustrated in FIG. 1A and described in detail above, or may be shorter if an alternative vascular access point is used, such as a brachial or subclavian artery puncture. Catheter shaft 82 comprises at least one lumen 126, which provides fluidic communication between the interior of electrode hood 83 and a fluid connector associated with the proximal terminal, not shown. Catheter shaft 82, also comprises a lumen 132 configured to house a coaxial cable 131, and may also comprise an additional lumen to house optional push wire 89. Catheter shaft 82 may be formed by extrusion of a polymeric compound commonly used in catheter making such as polyurethane, polyethylene, nylon, PEBAX®, etc. Catheter shaft 82 may also comprise a woven, knitted or coiled structure within its walls to provide torsional rigidity, while maintaining catheter shaft flexibility, which provides for high fidelity radial positioning of electrode hood 83 within a carotid artery. Electrode hood 83 comprises a hollow cylindrical structure, which is open at its proximal end, and closed with a substantially hemispherical distal bulkhead, as shown. Electrode hood 83 comprises a substantially electrically conductive inner surface 87, and a substantially electrically non-conductive outer surface 86. Electrode hood 83 comprises lateral fenestration 84 configured for fluidic communication between the interior and exterior of electrode hood 83. Ultrasonic transducer 88 is machined from a piezoelectric crystal, with a cylindrically concave surface 93 shown best in FIG. 10B, and a cylindrically convex surface 95 on the opposing side of concave surface 93 as shown in FIG. 10C. Cylindrically concave surface 93 has a radius of between 3 mm and 6 mm, which is also the focal length of ultrasonic transducer 88. Convex surface 95 is configured to closely approximate the inner diameter of electrode hood 83. Relief surface 96 is machined in convex surface 95. The length of ultrasonic transducer 88 is between approximately 6 mm as 12 mm, and is configured to reside within electrode hood 83 when assembled. Prior to assembly, and after machining, metallic coating 134 is applied to concave surface 93, and convex surface 95, with a masked region 133 between metalized concave surface 93, and metalized convex surface 95, which provides substantial electrical isolation between metalized concave surface 93 and metalized convex surface 95. The metalized coating may be applied by a sputtering process, a vapor deposition process, and may incorporate an electroplating process. The metal applied to surfaces 93 and 95 are selected for high electrical conductivity, nobility, and processability, which may include a gold or platinum alloy. During assembly, center conductor 129 of coaxial cable 131, is bonded to metalized concave surface 93 with solder or an electrically conductive adhesive. Convex metalized surface 95 is bonded to inner electrically conductive surface 87 of electrode hood 83 with solder, or an a electrically conductive adhesive, in a manner that a fluid tight air pocket is formed from profile relief surface 96, and the inner electrically conductive surface 87 of electrode hood 83. Ultrasonic transducer 88 is assembled into electrode hood 88 in diametric opposition to fenestration 84, as shown. Electrical contact 128, is soldered to outer conductor 130 of coaxial cable 131, and is positioned within the distal end of catheter shaft 82 for electrical contact and communication with inner electrically conductive surface 87 of electrode hood 83 when electrode hood 83 is assembled to the distal end of catheter shaft 82. After assembly, inner electrically conductive surface 87 of electrode hood 83, and metalized convex surface 95 of ultrasonic transducer 88 is in electrical communication with outer conductor 130 of coaxial cable 131, and concave metalized surface 93 is in electrical communication with center conductor 129 of coaxial cable 131. Lateral fenestration 84 has a length that approximates the length of ultrasonic transducer 88, and a width that approximates the width of ultrasonic transducer 88, which provides for a high percentage of the ultrasonic energy to be transmitted through fenestration 84, without significant energy loss due to reflection and diffraction from electrode hood's 83 structure. The air pocket formed between machined relief 96 of ultrasonic transducer 88, and electrically conductive inner surface 87 of electrode hood 83 forms a reflective ultrasonic energy barrier, resulting in a directional emission of ultrasonic energy from ultrasonic transducer 88 in the direction of fenestration 84. Electrode hood 83 outside diameter may approximate the outer diameter of catheter shaft 82, and has a wall thickness between approximately 0.005 inches and 0.025 inches. Electrode hood 83 may be formed from a metallic tube such as stainless steel, or a precious, and more radiopaque metal alloy such as a gold or platinum alloy, and insulated surface 86 comprising a polymeric or ceramic electrically insulating coating may then be applied to the metal substrate, thereby forming electrode cap 83. Electrode hood 83 is mounted on the distal end of catheter shaft 82 with lateral fenestration 84 radially positioned in diametric opposition to optional push wire 89, as shown. Representative push wire design and operation is illustrated in FIG. 8A, and FIG. 8B, and described in detail above. Proximal terminal, not shown, comprises a fluid connector in communication with the fluid lumen(s) 126 in catheter shaft 82, and is configured for connecting the fluid lumen(s) 126 of catheter shaft 82, and interior space of electrode hood 83 with a pressurized source of ionic liquid. Proximal terminal further comprises an electrical connector means configured for connecting inner conductor 129, and outer conductor 130 to an ultrasound console configured for ultrasonic energy tissue ablation, or stimulated harmonic sensing in conjunction with systemic administration of ultrasonic contrast agent, which will be elaborated in detail below, and a means for connecting outer conductor 130 to one pole of a radiofrequency energy generator configured for radiofrequency tissue ablation. The proximal terminal may also comprise a push wire 89 actuation mechanism, for which a representative description has been discussed above. ILE-CBA-DM catheter 81 may be configured for radiofrequency tissue ablation, simultaneous with ultrasonic tissue ablation, or serially. ILE-CBA-DM catheter 81 may also be configured to determine the effectiveness of carotid body ablation by measuring a change in ultrasonic stimulated harmonic emissions from the ablation tissue target. Those familiar in the art of ultrasonic transducer design, ultrasonic stimulated emission measurement, and ultrasonic tissue ablation are familiar with means for implementing the ultrasonic features disclosed here, therefore, no further description is warranted. A description for determining the effectiveness of a carotid body ablation my measuring a change in stimulated harmonic emissions is illustrated in FIGS. 17 and 18, and are described in detail below.

FIG. 11 is a schematic illustration of an ILE-CAB-F catheter 27 in situ during a carotid body ablation utilizing an ionic liquid stream. As depicted electrode hood 30 is positioned against the wall of the external carotid artery 12 at a position distal to the carotid bifurcation saddle 2, with forceps jaw 36 positioned against the medial wall of internal carotid artery 13. The axial position of outer sheath 28 determines the squeezing force between electrode hood 30, and forceps jaw 36. An adjustment of outer sheath 28 in the distal direction increases squeezing force between electrode hood 30 and forceps jaw 36, and an adjustment of outer sheath 28 in the proximal direction reduces squeezing force between electrode hood 30, and forceps jaw 36. Once electrode hood 30 is positioned, as shown, and outer sheath 28 is adjusted to obtain the optimal squeezing force between electrode hood 30 and forceps jaw 36, ionic liquid is introduced into the central lumen of ILE-CBA-F catheter 27, which displaces the arterial blood from the interior of electrode hood 30, and forms an electrical conduit between the arterial tissue immediately adjacent to electrode hood 30 and electrically conductive surface 33 of electrode hood 30. Radiofrequency voltage is then applied to the electrically conductive inner surface 33 of electrode hood 30, causing radiofrequency current to flow between electrically conductive surface 33, the ionic liquid, the perivascular tissue adjacent to electrode hood 30, and through the rest of the patient's body to the indifferent electrode, not shown. Due to the high radiofrequency current density in the vicinity of electrode hood 30, the perivascular tissue adjacent to electrode hood 30 is heated to ablative temperatures, resulting ablation zone 103, which substantially encompasses carotid body 99. Alternatively, forceps jaw 36, may be configured as an electrode, and connectable to a second pole of a radiofrequency energy generator, for a bipolar ablation configuration.

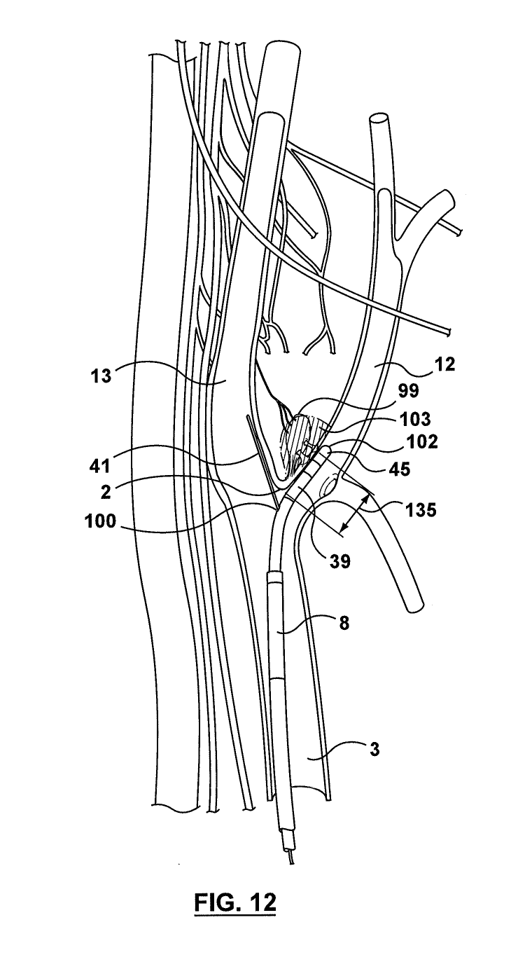

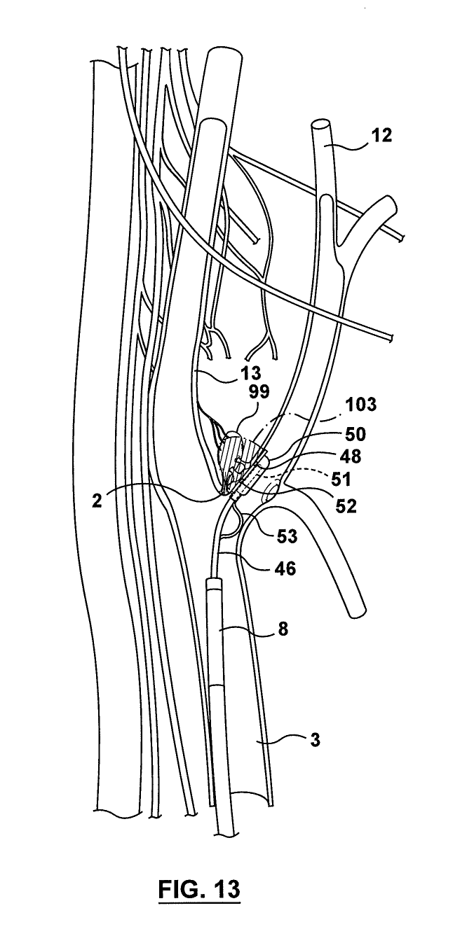

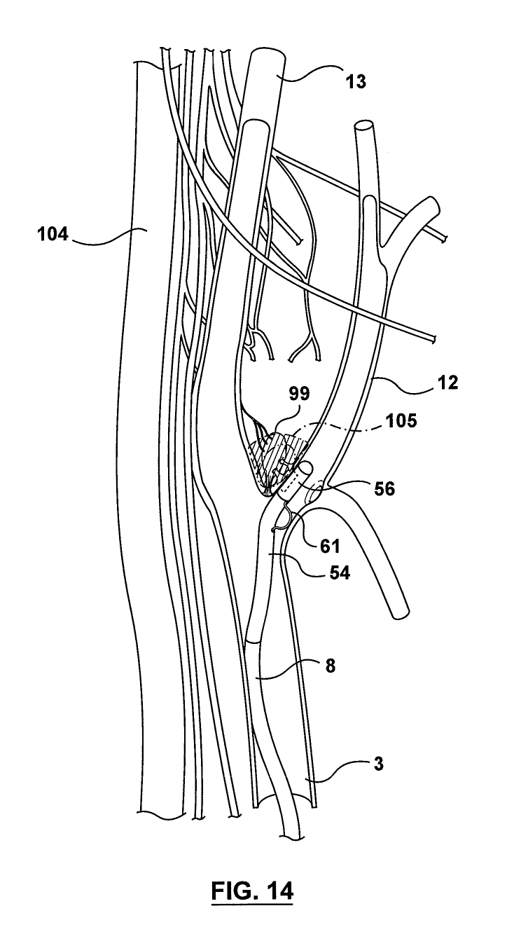

FIG. 12 is a schematic illustration of an ILE-CBA-SG catheter 39 in situ during a carotid body ablation utilizing an ionic liquid stream. As depicted electrode cap 45 is positioned against the wall of the external carotid artery 12 at a position distal to the carotid bifurcation saddle 2, and adjacent to carotid body 99. Guide wire 41 exiting side guide wire port 100 is positioned into the internal carotid artery 13. Guide wire 41 in conjunction with guide wire port 100 provide a means for positioning the electrode cap 45 against the wall of external carotid artery 12 at a determined distance 135 based on the distance between the distal tip 102 and the guide wire port 100. The force of contact between electrode cap 45 and the wall of the external carotid artery 12 can be influenced by the selection of the stiffness or diameter of the guide wire 41, the angle of exit of the guide wire 41, as well as the distance between distal tip 102 and guide wire port 100. Guide wire 41 may help to orient the direction of fenestrations 21 toward the carotid septum. Ablation zone 103 is depicted encompassing the periarterial space comprising the carotid body 99. Also depicted is the carotid access sheath 8 used for placement of the ILE-CBA-SG catheter 39 into the position shown. Alternatively, guide wire 47 may be positioned in external carotid artery 12, and electrode cap 45 may be positioned into the internal carotid artery 13, not shown.

FIG. 13 is a schematic illustration of ILE-CBA-B catheter 46 in situ during a carotid body ablation utilizing an ionic liquid stream. As depicted longitudinal fenestration 52 of balloon 48 is positioned against the wall of the external carotid artery 12 at a position distal to the carotid bifurcation saddle 2, and adjacent to carotid body 99. Push wire 53 is shown pressing longitudinal fenestration 52 of balloon 48 against the medial wall in external carotid artery 12, as shown. Ablation zone 103 is depicted encompassing the periarterial space comprising the carotid body 99. Also depicted is the carotid access sheath 8 used for placement of the ILE-CBA-B catheter 46 into the position shown. Alternatively, ILE-CBA-B catheter 46 may be inserted into internal carotid artery 13, and longitudinal fenestration 52 of balloon 48, may be pressed against the medial wall of internal carotid artery 13 adjacent to carotid body 99 by push wire 53, not shown.

FIG. 14 is a schematic illustration of ILE-CBA-ES catheter 62 in situ during a carotid body ablation utilizing an ionic liquid stream. As depicted elastic slit 58 of elastic membrane 57 is positioned against the wall of the external carotid artery 12 at a position distal to the carotid bifurcation saddle 2, and adjacent to carotid body 99. Push wire 61 is shown pressing elastic slit 58 of elastic membrane 57 against the medial wall in external carotid artery 12, as shown. Ablation zone 105 is depicted encompassing the periarterial space comprising the carotid body 99. Also depicted is the carotid access sheath 8 used for placement of the ILE-CBA-ES catheter 54 into the position shown. Alternatively, ILE-CBA-ES catheter 54 may be inserted into internal carotid artery 13, and elastic slit 58 of elastic membrane 57, may be pressed against the medial wall of internal carotid artery 13 adjacent to carotid body 99 by push wire 61, not shown.

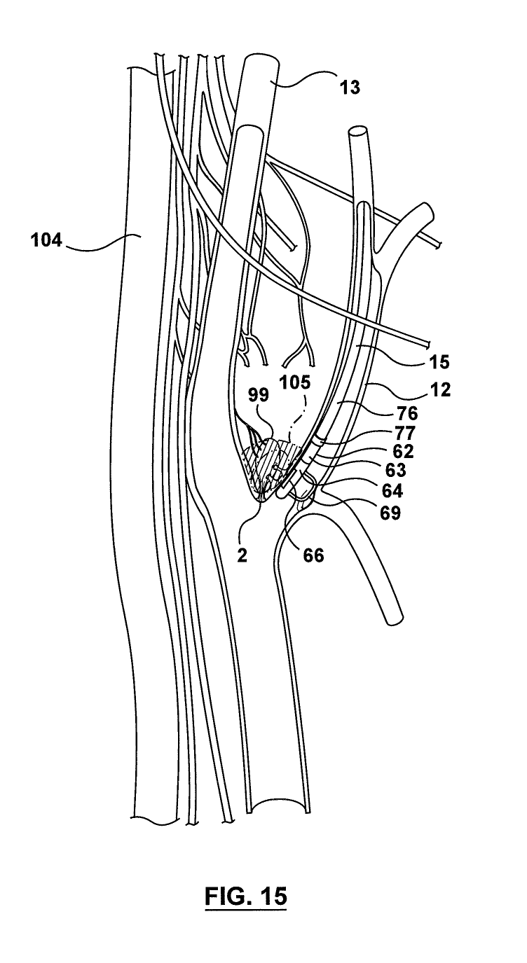

FIG. 15 is a schematic illustration of ILE-CBA-SC catheter 62, in situ, with access to the region of carotid body 99 from a superficial temporal artery puncture. Electrode cap 64 is being pushed against the wall of external carotid artery 12 immediately distal to carotid bifurcation 2 and immediately adjacent to carotid body 99. ILE-CBA-SC catheter 62 may be inserted into the vicinity of carotid bifurcation 2 using introducer sheath assembly 15 as shown, and previously described above, or without an introducer sheath by means of surgical cut-down of the superficial temporal artery and direct insertion of ILE-CBA-SC catheter 62 into the superficial temporal artery. Electrode cap 64 is advanced to the level of carotid body 99 under radiographic guidance. The radial position of fenestration 66 is determined by injection of a radiographic contrast medium through fluid connector, not shown, which exits fenestration 66 giving the user a fluoroscopic indication of the radial position of fenestrations 66. Catheter shaft is 63 is then rotated to radially position fenestration 66 towards carotid body 99. Push wire 69 is then extended using actuator 119, not shown pressing fenestration 66 of electrode cap 64 against the wall of external carotid artery 12.

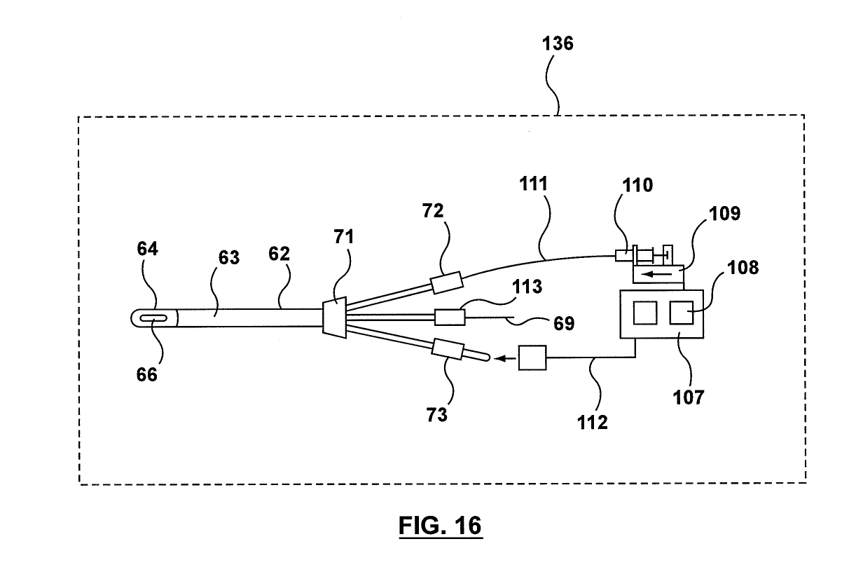

FIG. 16 is an illustration of a carotid body ablation system 136 configured for carotid body ablation utilizing an ionic liquid stream. Carotid body ablation system 136 comprises exemplary ILE-CBA-SC catheter 62, ablation console 107, ionic liquid pump 109, ionic liquid reservoir 110, electrical cable 112, and ionic liquid tubing set 111. As previously described, ILE-CBA-SC catheter 62 comprises electrode cap 64 and fenestration 66, catheter shaft 63, push wire 69, and proximal terminal 71 comprising fluid connector 72, Push wire terminal 113, and electrical connector 75. Control console 107 comprises a source of ablation energy, which in this example is radiofrequency energy, a user interface 108, that provides the user with a means for setting ablation parameters, activating an ablation, terminating an ablation, observing ablation parameters, and progress, and for proving the user with warnings and indications of the status of operations. In addition, console 107 may comprise a means for automatically controlling ionic liquid pump 109 during an ablation, and a means for the user to select ionic pump 109 operating parameters. Fluid connector 72 is connected to ionic liquid reservoir 110, which is depicted as a syringe, by ionic liquid tubing set 111. Ionic liquid reservoir 110 is inserted into ionic liquid pump 109, which provides the means for motivating ionic liquid flow through ILE-CBA-SC catheter 62, and through fenestration 66. Electrical connector 73 is connected to ablation console 107 by electrical cable 112. An indifferent electrode, or patient grounding pad, not shown, is also connected to ablation console 107 to compete the radiofrequency ablation circuit. ILE CBA-SC catheter 62 is inserted into an external carotid artery through a temporal artery puncture as previously described. Radiographic contrast agent is infused into ILE-CBA-SC catheter 62 through fluid connector 72 and out of fenestration 66 while the distal end of ILE-CBA-SC catheter 62 is observed using fluoroscopic imaging, which provides the user with an indication of the depth, and radial position of fenestration 66. Catheter shaft 63 is then rotated and axially position so that fenestration 66 is adjacent to, and at the depth of the target carotid body. Push wire 69 is advanced into ILE-CBA catheter 62 until fenestration 66 is pushed against the medial wall of the external carotid artery, then push wire 69 is secured in its actuated position using push wire terminal 113, which may comprise a Tuohy-Borst connector. Ablation parameters are selected using user interface, which may comprise a power setting between approximately 2 and 20 watts, and a time between approximately 10 and 120 seconds. Ionic liquid flow, which may comprise saline, or radiographic contrast medium, is then initiated by ionic liquid pump 109, displacing blood from electrode hood 64, and between fenestration 66 and the wall of the external carotid artery. Ablation console 107 is then activated using user interface 108 to apply RF current between the interior electrically conductive surface of electrode cap 64 to the indifferent electrode, not shown though the ionic liquid and the wall and periarterial tissue comprising the target carotid body. Upon completion of the RF energy application, carotid body function may then be assessed. If it determined that carotid body function is above a determined clinical threshold, then the ablation may be repeated at the same or different RF ablation parameters. It should be clearly understood that ILE-CBA-SC catheter 62 is exemplary, and that any previously or subsequently described catheter embodiment within this disclosure is within the scope of this invention. Also, console 107 may be configured for delivering an alternate ablation energy source including, but not limited to ultrasonic energy in conjunction with a catheter configured ultrasonic energy ablation as previously described. Ionic liquid pump 109 may comprise alternative means for pressurizing and controlling the flow of ionic liquid, including rotary pumps, and gravity motivation.

FIG. 17 is a graph of primary and harmonic acoustic return intensities prior to, and after a successful carotid body ablation with ILE-CBA-DM catheter 81, which comprises ultrasonic sensing capability in addition to RF ablation capability used in conjunction with a systemic administration of an ultrasonic contrast agent. Ultrasonic transducer 88 of ILE-CBA-DM catheter 81, previously described in detail, is configured to emit ultrasonic energy at a primary frequency and determined intensity into the perivascular tissue comprising a target carotid body, and to sense and measure return of ultrasonic energy from said perivascular tissues at the primary frequency, and at least the 1st harmonic frequency to the primary frequency. Ultrasonic contrast agents comprise micro-balloons comprising an outer shell, and a fluorocarbon gas, at size small enough to fully circulate through a patient's capillary vasculature. Ultrasonic contrast agent micro-balloons are highly elastic structures, and when excited by ultrasonic energy, vibrate at the primary ultrasonic frequency, and at harmonic and sub-harmonic frequencies, thereby emitting ultrasonic energy at harmonic and sub-harmonic frequencies to the primary frequency. Tissue without ultrasonic contrast agents, or with relatively low blood perfusion rates, weakly, or substantially do not emit at harmonic frequencies. The harmonic returned acoustic volume of ultrasonic energy by the micro-balloons is a function of the number of micro-balloon within the sensing field of the ultrasonic transducer. Since, during systemic administration of ultrasonic contrast agents within a body, the concentration of micro-balloons per unit of blood volume is substantially uniform, providing sufficient time has elapsed since the administration of the contrast agent. Since a carotid body is known to have the highest capillary blood perfusion rate of any organ in the body, by over an order of magnitude, the harmonic return signal from a healthy carotid body will greatly exceed the harmonic return signal from the periarterial tissue surrounding the carotid body. Following a successful carotid body ablation, the capillary perfusion associated with the carotid body is also substantially ablated, thereby greatly diminishing the harmonic return signals associated with the carotid body following a successful ablation. The change in harmonic return signals before, and after ablation may be quantified and used as indication of the effectiveness of the ablation. Furthermore, the ablation may be halted upon a determined reduction in harmonic emission, thereby reducing the chance of injury to adjacent vital structures. Intervening arterial blood between the ultrasonic transducer, and the periarterial tissue comprising a target carotid body will interfere the harmonic sensing of the periarterial tissue, therefore, the displacement of arterial blood from between the ultrasonic transducer and the arterial wall by an ionic liquid stream is a key requirement for using harmonic ultrasonic sensing of periarterial tissue as a means for determining the effectiveness of a carotid body ablation. FIG. 17 depicts the return acoustic intensity at the primary ultrasonic frequency, and the first two harmonic frequencies to the primary frequency, prior to, and following a successful carotid body ablation, showing no substantial change of the acoustic return at the primary ultrasonic frequency, but with a significant diminishment of acoustic return at the first and second harmonic frequencies, indicating a successful ablation of carotid body blood perfusion, and therefore function. The primary frequency may be between approximately 300 KHz and 3 MHz, and the acoustic intensity from the transducer may have a mechanical index between 0.3 and 1.3.

FIG. 18 is a graph of primary and harmonic acoustic intensities during a successful carotid body ablation with an ILE-CBA-DM catheter 81. The graph in FIG. 18 illustrates the change in change in acoustic harmonic return from perivascular tissue, as the ablation progresses, showing no substantial change in the primary ultrasonic frequency return. A continuous measurement of harmonic returns as depicted in FIG. 18 has an advantage over a before-and-after ablation acoustic harmonic measurement as described above by providing real-time feedback of the progression of a carotid body ablation. A lack of change in harmonic return emission may provide the user with an early indication of a hazardous condition, such a wrongly positioned ablation element. Also, a determination for the earliest possible termination of an ablation based on the change in acoustic harmonic returns may significantly reduce the risk of injury to adjacent vital structures.

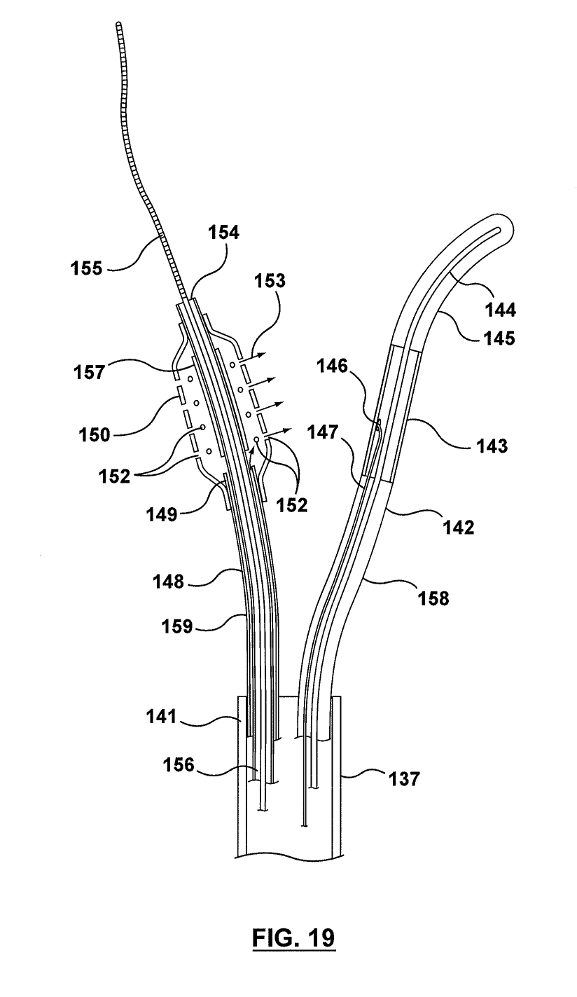

FIG. 19 is a schematic illustration of a Bipolar-Ionic Liquid Electrode-Carotid Body Ablation-Balloon Forceps (BP-ILE-CBA-BF) catheter 137. BP-ILE-CBA-BF catheter 137 comprises outer sheath 141, electrode arm 158, balloon arm 159 and a proximal terminal, not shown. Electrode arm 158 comprises electrode arm shaft 142, Electrode ring 143, shape form wire, 144, distal tip 145, electrical conductor 147, electrical connection 146 between electrical conductor 147 and electrode ring 143, with electrical conductor 146 providing electrical communication between electrode ring 143 and an electrical connector at the proximal terminal. Balloon arm 159 comprises balloon arm shaft 148, comprising ionic liquid lumen 149, and guidewire lumen 156, balloon 150 comprising fenestrations 152, balloon electrode 151 mounted within balloon 150 on balloon arm shaft 148 as shown, electrical conductor not shown, in electrical communication between balloon electrode 151, and an electrical connector at the proximal terminal. Balloon arm shaft is depicted with removable guidewire 155 residing within guidewire lumen 156. Electrode arm 158, and balloon arm are configured to be in a slidable relationship with outer sheath 141, and may be extended from outer sheath 141, may be withdrawn into outer sheath 137, and may be withdrawn from and inserted into outer sheath 141 proximal end (opposite end of that depicted), either together, or independently. The electrical connector at the proximal terminal, not shown is configured to connect ring electrode 143 to one pole of a radiofrequency energy generator, not shown, and balloon electrode 151 with the second pole of the radiofrequency energy generator, forming a bipolar radiofrequency ablation configuration between electrode ring 146, and balloon electrode 151. Pressurized ionic liquid 153 is infused through ionic liquid lumen 149, by means of an ionic liquid pump, not shown, and a fluid connector at the proximal terminal, also not shown. The pressurized ionic liquid enters balloon 150 under pressure, causing balloon 150 to inflate, and the ionic liquid 153 exits the balloon through fenestrations 152, and conducts radiofrequency current fenestrations 152 completing the radiofrequency ablation circuit between ring electrode 143, and balloon electrode 151. Shape form wire 144 comprises a wire made from a shape memory alloy such as Nitinol®, which forms the distal bend shown in distal tip 145 of electrode shaft 142, for atraumatic crossing of a carotid bifurcation from within a common carotid artery. Balloon 150 may comprise a compliant elastomeric structure formed from a silicone rubber compound, a urethane rubber compound, or a latex rubber compound. Alternatively, balloon 150 may be formed from a substantially non-compliant polymer, such as PET, or polyethylene. Those skilled in the art of balloon catheter design and construction are familiar means for forming and assembling balloons as described here within, therefore, no further explanation is warranted. Fenestrations 152, are configured to communicate pressurized ionic liquid from the interior of balloon 152 into the arterial space surrounding balloon 150, with a resistance to the flow of ionic liquid sufficient to cause a pressure within the interior of balloon 150 sufficient to inflate balloon 150. Fenestrations 152 may be formed within the wall of balloon 150 by puncturing the wall of balloon 150, without removing wall material, alternatively, fenestrations 152 may be machined into the wall of balloon 150, which may involve a laser machining operation. The combined cross sectional area of fenestration(s) 152 may be between approximately 0.2 square millimeters to 60 square millimeters. The combined cross sectional area of fenestrations 152 may be adjusted by adjusting the flow rate of ionic liquid through fenestrations 152. As the flow rate of ionic liquid is increased, the pressure within balloon 150 increases, causing the surface area of balloon 150, and cross sectional area of longitudinal fenestrations 152 to increase.

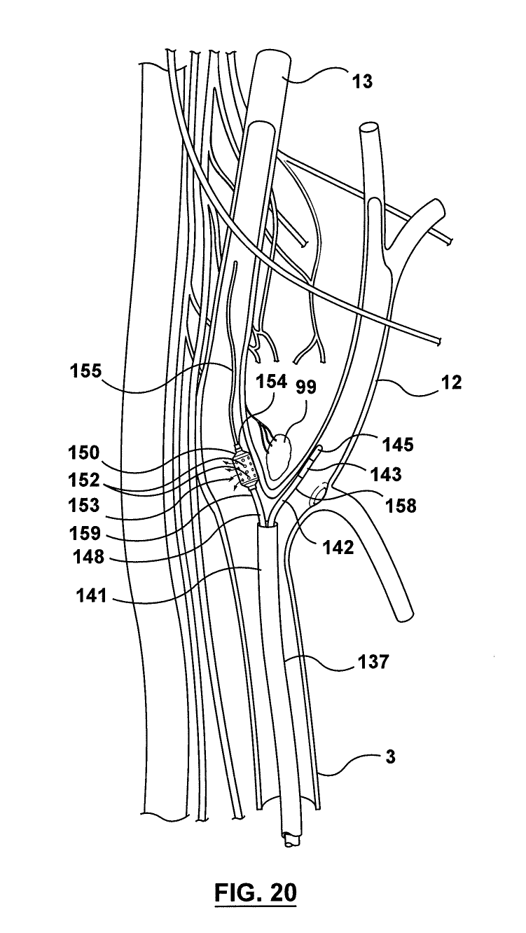

FIG. 20 is a schematic illustration of a BP-ILE-CBA-BF catheter 137 in situ immediately prior to a carotid body 99 ablation. As depicted, the distal end of outer sheath 141 is residing within common carotid artery 3, proximal to carotid bifurcation 2. Electrode arm 158 is shown residing within the proximal section of external carotid artery 12, with ring electrode 143 against the medial wall of external carotid artery 12 adjacent to carotid body 99, and balloon arm 159 residing within the proximal section of internal carotid artery 13, with balloon 150 against the medial wall of internal carotid artery 13 adjacent to carotid body 99. Alternatively, a balloon or virtual electrode may be in an internal carotid artery but not in contact with the vessel wall, directing an ionic liquid stream to contact the vessel wall. Also depicted is balloon 150 being inflated by pressurized ionic liquid 153 shown exiting balloon 150 through fenestrations 152. The axial position of outer 141 determines the pinching force applied to carotid bifurcation 2 by electrode arm 158, and balloon arm 159. The further distal outer sheath 141 is, the greater said pinching force.

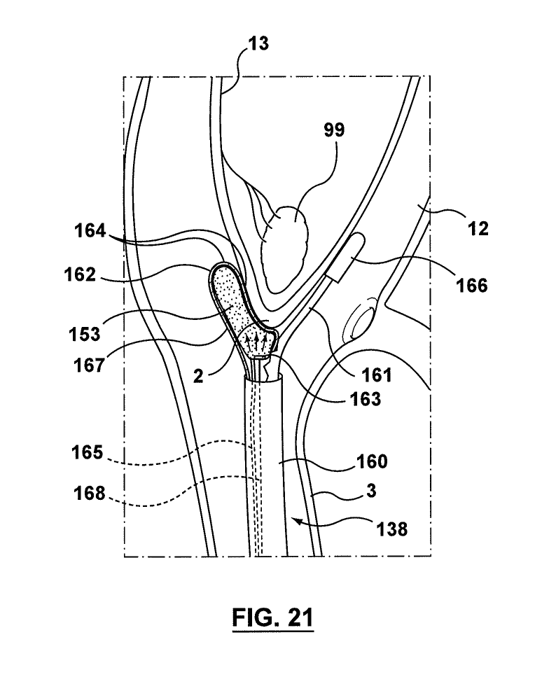

FIG. 21 is a schematic illustration of Bipolar-Ionic Liquid Electrode-Carotid Body Ablation-Bladder Electrode catheter 138 in situ immediately prior to a carotid body ablation. As depicted, the distal end of outer sheath 160 is residing within common carotid artery 3, proximal to carotid bifurcation 2. Electrode arm 161 is shown residing within the proximal section of external carotid artery 12, with electrode 166 against the medial wall of external carotid artery 12 adjacent to carotid body 99, and bladder electrode straddling carotid bifurcation 2 and the medial wall of internal carotid artery 13 as shown. Also depicted is bladder electrode 162 being inflated by pressurized ionic liquid 153 shown exiting bladder electrode 162 through bladder perforations 164. Electrode 166 and electrode arm 161 are functionally and structurally equivalent to electrode arm 158 of BP-ILE-CBA-BF catheter 137 described above. Bladder electrode 162 comprises bladder 167, and electrode tube 163. Bladder 167 is mounted at the distal end of ionic liquid lumen 168 within outer sheath 160, or at the distal end of a catheter residing within outer sheath 160, not shown. Electrode tube 163 is mounted within ionic liquid lumen 168, and comprise a hypodermic tube press fit, or, glued into the distal end ionic liquid lumen 168. Electrode tube is in electrical communication with an electrical connector at the proximal terminal not shown by electrical conductor 165. Bladder 167 may comprise a compliant elastomeric structure formed from a silicone rubber compound, a urethane rubber compound, or a latex rubber compound. Alternatively, bladder 167 may be formed from a substantially non-compliant polymer, such as PET, or polyethylene. Those skilled in the art of balloon catheter design and construction are familiar means for forming and assembling a bladder as depicted here within, therefore, no further explanation is warranted. Bladder wall perforations 164, are configured to communicate pressurized ionic liquid from the interior of bladder 167 into the arterial space surrounding bladder 167, with a resistance to the flow of ionic liquid sufficient to cause a pressure within the interior of bladder 167 sufficient to inflate bladder 167. Perforations 164 may be formed within the wall of bladder 167 by puncturing the wall of bladder 167, without removing wall material, alternatively, perforations 167 may be machined into the wall of bladder 167, which may involve a laser machining operation. The combined cross sectional area of perforations 164 may be between approximately 0.2 square millimeters to 6 square millimeters. The combined cross sectional area of perforations 164 may be adjusted by adjusting the flow rate of ionic liquid through perforations 164. As the flow rate of ionic liquid is increased, the pressure within bladder 167 increases, causing the surface area of bladder 167, and cross sectional area of fenestrations 164 to increase. Electrode tube 163 is in electrical communication with ionic fluid 153 within ionic fluid lumen 168. The electrical connector at the proximal terminal, not shown is configured to connect electrode 166 to one pole of a radiofrequency energy generator, not shown, and electrode tube 163 with the second pole of the radiofrequency energy generator, forming a bipolar radiofrequency ablation configuration between electrode 166, and electrode tube 163. Pressurized ionic liquid 153 is infused through ionic liquid lumen 168, by means of an ionic liquid pump, not shown, and a fluid connector at the proximal terminal, also not shown. The pressurized ionic liquid enters bladder 162 under pressure, causing bladder 162 to inflate, and the ionic liquid 153 exits bladder 167 through perforations 164, and conducts radiofrequency current through perforations 164 completing the radiofrequency ablation circuit between electrode 166, and electrode tube 163.

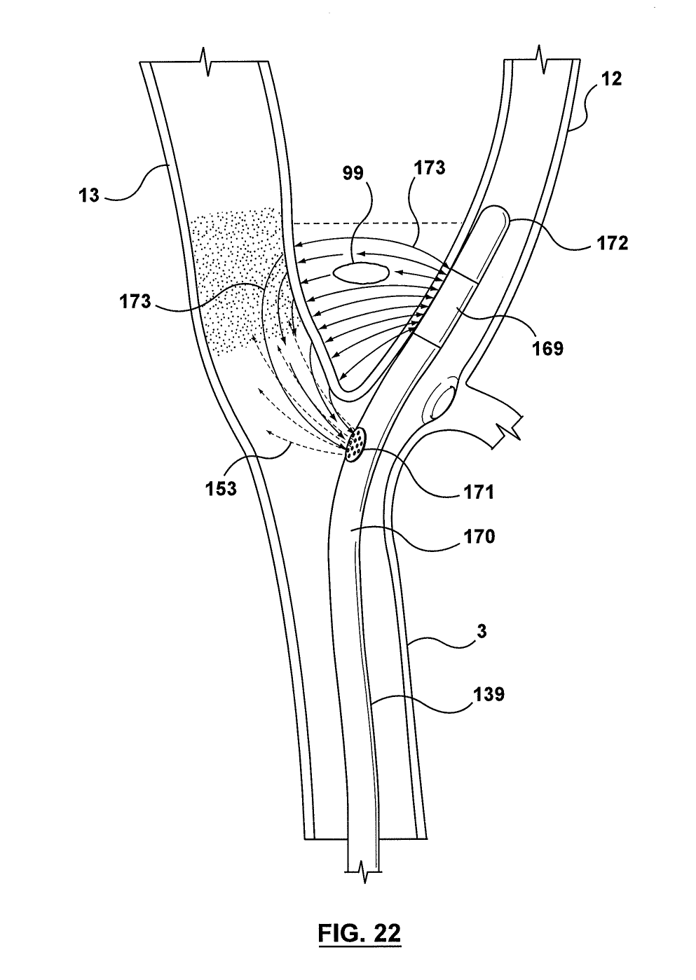

FIG. 22 is a schematic illustration of Bipolar-Ionic Liquid Electrode-Carotid Body Ablation-Side Port Electrode (BP-ILE-CBA-SPE) catheter 139 in situ during a carotid body ablation. BP-ILE-CBA-SPE catheter 139 comprises catheter shaft 170, with a distal end 172, and proximal end, not shown, ring electrode 169, and side port electrode 171, and proximal terminal, not shown. Catheter shaft 170 comprises an ionic fluid lumen, not shown, which terminates at side port electrode 171, and is in fluidic communication with a fluid connector at the proximal terminal; an electrical conductor in electrical communication between electrode 169 and an electrical connector at the proximal terminal, and a second electrical conductor in electrical communication with side port electrode 171 and a second contact of the electrical connector at the proximal terminal. The electrical connector, not shown is configured to connect electrode 169 to one pole on a radiofrequency energy generator, not shown, and side port electrode 171 to the second pole of the radiofrequency energy generator, creating a bipolar electrode pair. Side port electrode comprises a metallic screen configured for electrical communication with ionic liquid 153 as it exits the ionic liquid lumen. Side port electrode 171 is configured with a lateral location as shown, and is positioned proximal to electrode 169 so the majority of ionic liquid 153 exiting side port electrode 171 will enter the distal blood stream of internal carotid artery 13 as illustrated. Ionic liquid 153 may comprise a hypertonic saline, which will have a higher electrical conductivity than arterial blood, and create a path of least resistance for radiofrequency current 172 in internal carotid artery 13. BP-ILE-CBA-SPE catheter 139 may be positioned as shown in external carotid artery 12, or in a mirrored configuration with distal tip 172 positioned within the internal carotid artery 13 with side port electrode 171 directing ionic liquid into the distal blood stream of external carotid artery 12. BP-ILE-CBA-SPE catheter 139 may comprise a steerable distal tip 172 to facilitate positioning as shown, or may comprise other means for positioning distal tip 172 as previous described above for other catheter embodiments. The radial position of side port electrode 171 may be fluoroscopically determined by injection of radiographic contrast agent through ionic liquid lumen, and side port electrode 171.

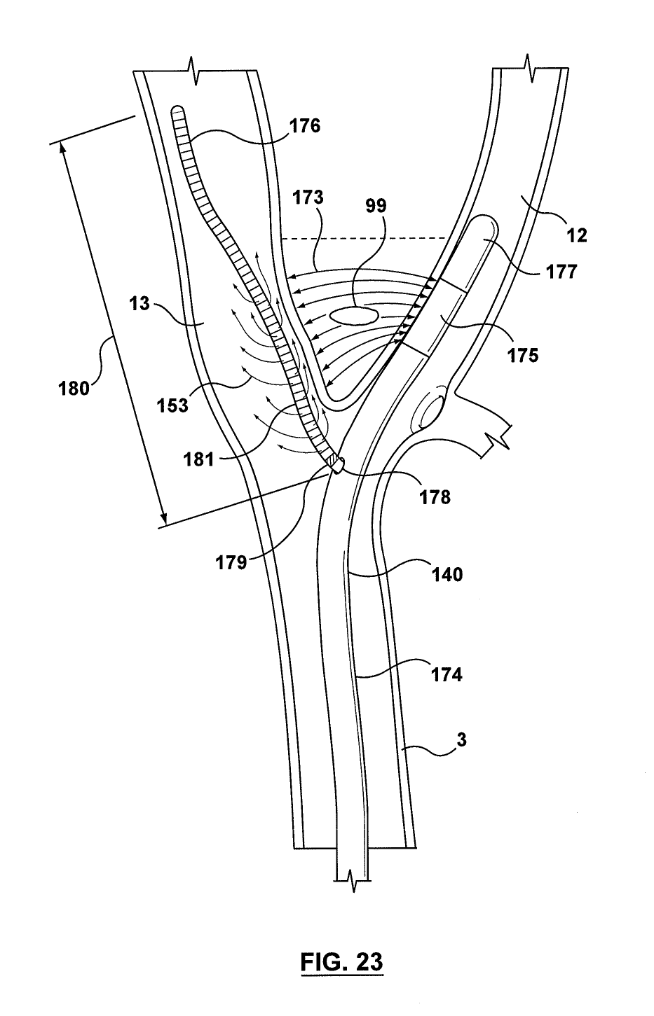

FIG. 23 a schematic illustration of Bipolar-Ionic Liquid Electrode-Carotid Body Ablation-Side Electrode Guidewire (BP-ILE-CBA-SEG) catheter 140 in situ during a carotid body ablation. BP-ILE-CBA-SEG catheter 140 comprises catheter shaft 174, with a distal end 177, and proximal end, not shown, ring electrode 175, and side port 178, weeping guidewire electrode 176, and proximal terminal, not shown. Catheter shaft 174 comprises a guide wire lumen, not shown, which terminates at side port 178, and is in communication with a guidewire connector at the proximal terminal, which may comprise Tuohy-Borst connector; an electrical conductor in electrical communication between ring electrode 175 and an electrical connector at the proximal terminal. Weeping guidewire electrode 176 comprises a metallic wire coil 181, with a hollow center section, and an outer watertight coating 179 except for the distal uncoated segment 180. The metal wire coil 181 is in electrical communication with a second conductor of the electrical connector at the proximal terminal. The hollow central center section of metal wire coil 181 is in fluidic communication with a fluid connector at the proximal terminal. The electrical connector, not shown is configured to connect electrode 175 to one pole of a radiofrequency energy generator, not shown, and metal wire coil 181 to the second pole of the radiofrequency energy generator, creating a bipolar electrode pair. Fluid connector, not shown is configured for connecting the hollow center section of metallic wire coil 181 to a source of pressurized ionic liquid 153. Metallic wire coil 181 is configured for electrical communication with ionic liquid 153 as it traverses the hollow center section of metallic metal coil 181. Side port 178 is configured with a lateral location as shown, and is positioned proximal to electrode 175 so weeping guidewire electrode may be positioned within internal carotid artery 13 as shown. Ionic liquid 153 may comprise a hypertonic saline, which will have a higher electrical conductivity than arterial blood, and create a path of least resistance for radiofrequency current 173 in internal carotid artery 13. BP-ILE-CBA-SEG catheter 140 may be positioned as shown in external carotid artery 12, or in a mirrored configuration with distal tip 177 positioned within the internal carotid artery with side port 178 directing weeping guidewire electrode 176 into external carotid artery 12. BP-ILE-CBA-SEG catheter 140 may comprise a steerable distal tip to facilitate positioning as shown, or may comprise other means for positioning as shown, as previous described above for the previously described catheter embodiments.

### Methods of Therapy:

An ablation energy source (e.g. energy field generator) may be located external to the patient. The generator may include computer controls to automatically or manually adjust frequency and strength of the energy applied to the catheter, timing and period during which energy is applied, and safety limits to the application of energy. It should be understood that embodiments of energy delivery electrodes described hereinafter may be electrically connected to the generator even though the generator is not explicitly shown or described with each embodiment.

An ablated tissue lesion at or near the carotid body may be created by the application of ablation energy from an ablation element in a vicinity of a distal end of the carotid body modulation device. The ablated tissue lesion may disable the carotid body or may suppress the activity of the carotid body or interrupt conduction of afferent nerve signals from a carotid body to sympathetic nervous system. The disabling or suppression of the carotid body reduces the responsiveness of the glomus cells to changes of blood gas composition and effectively reduces activity of afferent carotid body nerves or the chemoreflex gain of the patient.

A method in accordance with a particular embodiment includes ablating at least one of a patient's carotid bodies based at least in part on identifying the patient as having a sympathetically mediated disease such as cardiac, metabolic, or pulmonary disease such as hypertension, insulin resistance, diabetes, pulmonary hypertension, drug resistant hypertension (e.g. refractory hypertension), congestive heart failure (CHF), or dyspnea from heart failure or pulmonary disease causes.

A procedure may include diagnosis, selection based on diagnosis, further screening (e.g. baseline assessment of chemosensitivity), treating a patient based at least in part on diagnosis or further screening via a chemoreceptor (e.g. carotid body) ablation procedure such as one of the embodiments disclosed. Additionally, following ablation a method of therapy may involve conducting a post-ablation assessment to compare with the baseline assessment and making decisions based on the assessment (e.g. adjustment of drug therapy, re-treat in new position or with different parameters, or ablate a second chemoreceptor if only one was previously ablated).

A carotid body modulation procedure may comprise the following steps or a combination thereof: patient sedation, locating a target peripheral chemoreceptor, visualizing a target peripheral chemoreceptor (e.g. carotid body), confirming a target ablation site is or is proximate a peripheral chemoreceptor, confirming a target ablation site is safely distant from important non-target nerve structures that are preferably protected (e.g. hypoglossal, sympathetic and vagus nerves), providing stimulation (e.g. electrical, mechanical, chemical) to a target site or target peripheral chemoreceptor prior to, during or following an ablation step, monitoring physiological responses to said stimulation, providing temporary nerve block to a target site prior to an ablation step, monitoring physiological responses to said temporary nerve block, anesthetizing a target site, protecting the brain from potential embolism, thermally protecting an arterial or venous wall (e.g. carotid artery, jugular vein) or a medial aspect of an intercarotid septum or non-target nerve structures, ablating a target site (e.g. peripheral chemoreceptor), monitoring ablation parameters (e.g. temperature, pressure, duration, blood flow in a carotid artery), monitoring physiological responses during ablation and arresting ablation if unsafe or unwanted physiological responses occur before collateral nerve injury becomes permanent, confirming a reduction of chemoreceptor activity (e.g. chemosensitivity, HR, blood pressure, ventilation, sympathetic nerve activity) during or following an ablation step, removing a ablation device, conducting a post-ablation assessment, repeating any steps of the chemoreceptor ablation procedure on another peripheral chemoreceptor in the patient.

Patient screening, as well as post-ablation assessment may include physiological tests or gathering of information, for example, chemoreflex sensitivity, central sympathetic nerve activity, heart rate, heart rate variability, blood pressure, ventilation, production of hormones, peripheral vascular resistance, blood pH, blood PCO2, degree of hyperventilation, peak VO2, VE/VCO2 slope. Directly measured maximum oxygen uptake (more correctly pVO2 in heart failure patients) and index of respiratory efficiency VE/VCO2 slope has been shown to be a reproducible marker of exercise tolerance in heart failure and provide objective and additional information regarding a patient's clinical status and prognosis.

A method of therapy may include electrical stimulation of a target region, using a stimulation electrode, to confirm proximity to a carotid body. For example, a stimulation signal having a 1-10 milliamps (mA) pulse train at about 20 to 40 Hz with a pulse duration of 50 to 500 microseconds (μs) that produces a positive carotid body stimulation effect may indicate that the stimulation electrode is within sufficient proximity to the carotid body or nerves of the carotid body to effectively ablate it. A positive carotid body stimulation effect could be increased blood pressure, heart rate, or ventilation concomitant with application of the stimulation. These variables could be monitored, recorded, or displayed to help assess confirmation of proximity to a carotid body. A catheter-based technique, for example, may have a stimulation electrode proximal to the ablation element used for ablation. Alternatively, the ablation element itself may also be used as a stimulation electrode. Alternatively, an energy delivery element that delivers a form of ablative energy that is not electrical, such as a cryogenic ablation applicator, may be configured to also deliver an electrical stimulation signal as described earlier. Yet another alternative embodiment comprises a stimulation electrode that is distinct from an ablation element. For example, during a surgical procedure a stimulation probe can be touched to a suspected carotid body that is surgically exposed. A positive carotid body stimulation effect could confirm that the suspected structure is a carotid body and ablation can commence. Physiological monitors (e.g. heart rate monitor, blood pressure monitor, blood flow monitor, MSNA monitor) may communicate with a computerized stimulation generator, which may also be an ablation generator, to provide feedback information in response to stimulation. If a physiological response correlates to a given stimulation the computerized generator may provide an indication of a positive confirmation.

Alternatively or in addition a drug known to excite the chemo sensitive cells of the carotid body can be injected directly into the carotid artery or given systemically into patients vein or artery in order to elicit hemodynamic or respiratory response. Examples of drugs that may excite a chemoreceptor include nicotine, atropine, Doxapram, Almitrine, hyperkalemia, Theophylline, adenosine, sulfides, Lobeline, Acetylcholine, ammonium chloride, methylamine, potassium chloride, anabasine, coniine, cytosine, acetaldehyde, acetyl ester and the ethyl ether of i-methylcholine, Succinylcholine, Piperidine, monophenol ester of homo-iso-muscarine and acetylsalicylamides, alkaloids of veratrum, sodium citrate, adenosinetriphosphate, dinitrophenol, caffeine, theobromine, ethyl alcohol, ether, chloroform, phenyldiguanide, sparteine, coramine (nikethamide), metrazol (pentylenetetrazol), iodomethylate of dimethylaminomethylenedioxypropane, ethyltrimethylammoniumpropane, trimethylammonium, hydroxytryptamine, papaverine, neostigmine, acidity.

A method of therapy may further comprise applying electrical or chemical stimulation to the target area or systemically following ablation to confirm a successful ablation. Heart rate, blood pressure or ventilation may be monitored for change or compared to the reaction to stimulation prior to ablation to assess if the targeted carotid body was ablated. Post-ablation stimulation may be done with the same apparatus used to conduct the pre-ablation stimulation. Physiological monitors (e.g. heart rate monitor, blood pressure monitor, blood flow monitor, MSNA monitor) may communicate with a computerized stimulation generator, which may also be an ablation generator, to provide feedback information in response to stimulation. If a physiological response correlated to a given stimulation is reduced following an ablation compared to a physiological response prior to the ablation, the computerized generator may provide an indication ablation efficacy or possible procedural suggestions such as repeating an ablation, adjusting ablation parameters, changing position, ablating another carotid body or chemosensor, or concluding the procedure.

The devices described herein may also be used to temporarily stun or block nerve conduction via electrical neural blockade. A temporary nerve block may be used to confirm position of an ablation element prior to ablation. For example, a temporary nerve block may block nerves associated with a carotid body, which may result in a physiological effect to confirm the position may be effective for ablation. Furthermore, a temporary nerve block may block important non-target nerves such as vagal, hypoglossal or sympathetic nerves that are preferably avoided, resulting in a physiological effect (e.g. physiological effects may be noted by observing the patient's eyes, tongue, throat or facial muscles or by monitoring patient's heart rate and respiration). This may alert a user that the position is not in a safe location. Likewise absence of a physiological effect indicating a temporary nerve block of such important non-target nerves in combination with a physiological effect indicating a temporary nerve block of carotid body nerves may indicate that the position is in a safe and effective location for carotid body modulation.

Important nerves may be located in proximity of the target site and may be inadvertently and unintentionally injured. Neural stimulation or blockade can help identify that these nerves are in the ablation zone before the irreversible ablation occurs. These nerves may include the following:

Vagus Nerve Bundle—The vagus is a bundle of nerves that carry separate functions, for example a) branchial motor neurons (efferent special visceral) which are responsible for swallowing and phonation and are distributed to pharyngeal branches, superior and inferior laryngeal nerves; b) visceral motor (efferent general visceral) which are responsible for involuntary muscle and gland control and are distributed to cardiac, pulmonary, esophageal, gastric, celiac plexuses, and muscles, and glands of the digestive tract; c) visceral sensory (afferent general visceral) which are responsible for visceral sensibility and are distributed to cervical, thoracic, abdominal fibers, and carotid and aortic bodies; d) visceral sensory (afferent special visceral) which are responsible for taste and are distributed to epiglottis and taste buds; e) general sensory (afferent general somatic) which are responsible for cutaneous sensibility and are distributed to auricular branch to external ear, meatus, and tympanic membrane. Dysfunction of the vagus may be detected by a) vocal changes caused by nerve damage (damage to the vagus nerve can result in trouble with moving the tongue while speaking, or hoarseness of the voice if the branch leading to the larynx is damaged); b) dysphagia due to nerve damage (the vagus nerve controls many muscles in the palate and tongue which, if damaged, can cause difficulty with swallowing); c) changes in gag reflex (the gag reflex is controlled by the vagus nerve and damage may cause this reflex to be lost, which can increase the risk of choking on saliva or food); d) hearing loss due to nerve damage (hearing loss may result from damage to the branch of the vagus nerve that innervates the concha of the ear): e) cardiovascular problems due to nerve damage (damage to the vagus nerve can cause cardiovascular side effects including irregular heartbeat and arrhythmia); or f) digestive problems due to nerve damage (damage to the vagus nerve may cause problems with contractions of the stomach and intestines, which can lead to constipation).

Superior Laryngeal Nerve—the superior laryngeal nerve is a branch of the vagus nerve bundle. Functionally, the superior laryngeal nerve function can be divided into sensory and motor components. The sensory function provides a variety of afferent signals from the supraglottic larynx. Motor function involves motor supply to the ipsilateral cricothyroid muscle. Contraction of the cricothyroid muscle tilts the cricoid lamina backward at the cricothyroid joint causing lengthening, tensing and adduction of vocal folds causing an increase in the pitch of the voice generated. Dysfunction of the superior laryngeal nerve may change the pitch of the voice and causes an inability to make explosive sounds. A bilateral palsy presents as a tiring and hoarse voice.

Cervical Sympathetic Nerve—The cervical sympathetic nerve provides efferent fibers to the internal carotid nerve, external carotid nerve, and superior cervical cardiac nerve. It provides sympathetic innervation of the head, neck and heart. Organs that are innervated by the sympathetic nerves include eyes, lacrimal gland and salivary glands. Dysfunction of the cervical sympathetic nerve includes Horner's syndrome, which is very identifiable and may include the following reactions: a) partial ptosis (drooping of the upper eyelid from loss of sympathetic innervation to the superior tarsal muscle, also known as Milflees muscle); b) upside-down ptosis (slight elevation of the lower lid); c) anhidrosis (decreased sweating on the affected side of the face); d) miosis (small pupils, for example small relative to what would be expected by the amount of light the pupil receives or constriction of the pupil to a diameter of less than two millimeters, or asymmetric, one-sided constriction of pupils); e) enophthalmos (an impression that an eye is sunken in); f) loss of ciliospinal reflex (the ciliospinal reflex, or pupillary-skin reflex, consists of dilation of the ipsilateral pupil in response to pain applied to the neck, face, and upper trunk. If the right side of the neck is subjected to a painful stimulus, the right pupil dilates about 1-2 mm from baseline. This reflex is absent in Homer's syndrome and lesions involving the cervical sympathetic fibers.)

### Visualization:

An optional step of visualizing internal structures (e.g. carotid body or surrounding structures) may be accomplished using one or more non-invasive imaging modalities, for example fluoroscopy, radiography, arteriography, computer tomography (CT), computer tomography angiography with contrast (CTA), magnetic resonance imaging (MRI), or sonography, or minimally invasive techniques (e.g. IVUS, endoscopy, optical coherence tomography, ICE). A visualization step may be performed as part of a patient assessment, prior to an ablation procedure to assess risks and location of anatomical structures, during an ablation procedure to help guide an ablation device, or following an ablation procedure to assess outcome (e.g. efficacy of the ablation). Visualization may be used to: (a) locate a carotid body, (b) locate important non-target nerve structures that may be adversely affected, or (c) locate, identify and measure arterial plaque.

Endovascular (for example transfemoral) arteriography of the common carotid and then selective arteriography of the internal and external carotids may be used to determine a position of a catheter tip at a carotid bifurcation. Additionally, ostia of glomic arteries (these arteries may be up to 4 mm long and arise directly from the main parent artery) can be identified by dragging the dye injection catheter and releasing small amounts (“puffs”) of dye. If a glomic artery is identified it can be cannulated by a guide wire and possibly further cannulated by small caliber catheter. Direct injection of dye into glomic arteries can further assist the interventionalist in the ablation procedure. It is appreciated that the feeding glomic arteries are small and microcatheters may be needed to cannulate them.

Alternatively, ultrasound visualization may allow a physician to see the carotid arteries and even the carotid body. Another method for visualization may consist of inserting a small needle (e.g. 22 Gauge) with sonography or computer tomography (CT) guidance into or toward the carotid body. A wire or needle can be left in place as a fiducial guide, or contrast can be injected into the carotid body. Runoff of contrast to the jugular vein may confirm that the target is achieved.

Computer Tomography (CT) and computer tomography angiography (CTA) may also be used to aid in identifying a carotid body. Such imaging could be used to help guide an ablation device to a carotid body.

Ultrasound visualization (e.g. sonography) is an ultrasound-based imaging technique used for visualizing subcutaneous body structures including blood vessels and surrounding tissues. Doppler ultrasound uses reflected ultrasound waves to identify and display blood flow through a vessel. Operators typically use a hand-held transducer/transceiver placed directly on a patient's skin and aimed inward directing ultrasound waves through the patient's tissue. Ultrasound may be used to visualize a patient's carotid body to help guide an ablation device. Ultrasound can be also used to identify atherosclerotic plaque in the carotid arteries and avoid disturbing and dislodging such plaque.

Visualization and navigation steps may comprise multiple imaging modalities (e.g. CT, fluoroscopy, ultrasound) superimposed digitally to use as a map for instrument positioning. Superimposing borders of great vessels such as carotid arteries can be done to combine images.

Responses to stimulation at different coordinate points can be stored digitally as a 3-dimensional or 2-dimensional orthogonal plane map. Such an electric map of the carotid bifurcation showing points, or point coordinates that are electrically excitable such as baroreceptors, baroreceptor nerves, chemoreceptors and chemoreceptor nerves can be superimposed with an image (e.g. CT, fluoroscopy, ultrasound) of vessels. This can be used to guide the procedure, and identify target areas and areas to avoid.

In addition, as noted above, it should be understood that a device providing therapy can also be used to locate a carotid body as well as to provide various stimuli (electrical, chemical, other) to test a baseline response of the carotid body chemoreflex (CBC) or carotid sinus baroreflex (CSB) and measure changes in these responses after therapy or a need for additional therapy to achieve the desired physiological and clinical effects.

### Patient Selection and Assessment:

In an embodiment, a procedure may comprise assessing a patient to be a plausible candidate for carotid body modulation. Such assessment may involve diagnosing a patient with a sympathetically mediated disease (e.g. MSNA microneurography, measure of cataclomines in blood or urine, heart rate, or low/high frequency analysis of heart rate variability may be used to assess sympathetic tone). Patient assessment may further comprise other patient selection criteria, for example indices of high carotid body activity (i.e. carotid body hypersensitivity or hyperactivity) such as a combination of hyperventilation and hypocarbia at rest, high carotid body nerve activity (e.g. measured directly), incidence of periodic breathing, dyspnea, central sleep apnea elevated brain natriuretic peptide, low exercise capacity, having cardiac resynchronization therapy, atrial fibrillation, ejection fraction of the left ventricle, using beta blockers or ACE inhibitors.

Patient selection may involve non-invasive visualization such as CTA or MRI to identify location of a carotid body. For example, if the patient does not have at least one carotid body that is sufficiently within an intercarotid septum the patient may be ineligible for a CBM procedure that targets an intercarotid septum. Another example of patient selection using non-invasive visualization may involve excluding patients having large risk of dislodging plaque into an internal carotid artery.

Patient assessment may further involve selecting patients with high peripheral chemosensitivity (e.g. a respiratory response to hypoxia normalized to the desaturation of oxygen greater than or equal to about 0.7 l/min/min SpO₂), which may involve characterizing a patient's chemoreceptor sensitivity, reaction to temporarily blocking carotid body chemoreflex, or a combination thereof.

Although there are many ways to measure chemosensitivity they can be divided into (a) active provoked response and (b) passive monitoring. Active tests can be done by inducing intermittent hypoxia (such as by taking breaths of nitrogen or CO₂ or combination of gases) or by rebreathing air into and from a 4 to 10 liter bag. For example: a hypersensitive response to a short period of hypoxia measured by increase of respiration or heart rate may provide an indication for therapy. Ablation or significant reduction of such response could be indicative of a successful procedure. Also, electrical stimulation, drugs and chemicals (e.g. dopamine, lidocane) exist that can block or excite a carotid body when applied locally or intravenously.

The location and baseline function of the desired area of therapy (including the carotid and aortic chemoreceptors and baroreceptors and corresponding nerves) may be determined prior to therapy by application of stimuli to the carotid body or other organs that would result in an expected change in a physiological or clinical event such as an increase or decrease in SNS activity, heart rate or blood pressure. These stimuli may also be applied after the therapy to determine the effect of the therapy or to indicate the need for repeated application of therapy to achieve the desired physiological or clinical effect(s). The stimuli can be either electrical or chemical in nature and can be delivered via the same or another catheter or can be delivered separately (such as injection of a substance through a peripheral IV to affect the CBC that would be expected to cause a predicted physiological or clinical effect).

A baseline stimulation test may be performed to select patients that may benefit from a carotid body modulation procedure. For example, patients with a high peripheral chemosensitivity gain (e.g. greater than or equal to about two standard deviations above an age matched general population chemosensitivity, or alternatively above a threshold peripheral chemosensitivity to hypoxia of 0.5 or 0.7 ml/min/% O2) may be selected for a carotid body modulation procedure. A prospective patient suffering from a cardiac, metabolic, or pulmonary disease (e.g. hypertension, CHF, diabetes) may be selected. The patient may then be tested to assess a baseline peripheral chemoreceptor sensitivity (e.g. minute ventilation, tidal volume, ventilator rate, heart rate, or other response to hypoxic or hypercapnic stimulus). Baseline peripheral chemosensitivity may be assessed using tests known in the art which involve inhalation of a gas mixture having reduced O₂ content (e.g. pure nitrogen, CO₂, helium, or breathable gas mixture with reduced amounts of O₂ and increased amounts of CO₂) or rebreathing of gas into a bag. Concurrently, the patient's minute ventilation or initial sympathetically mediated physiologic parameter such as minute ventilation or HR may be measured and compared to the O₂ level in the gas mixture. Tests like this may elucidate indices called chemoreceptor setpoint and gain. These indices are indicative of chemoreceptor sensitivity. If the patient's chemosensitivity is not assessed to be high (e.g. less than about two standard deviations of an age matched general population chemosensitivity, or other relevant numeric threshold) then the patient may not be a suitable candidate for a carotid body modulation procedure. Conversely, a patient with chemoreceptor hypersensitivity (e.g. greater than or equal to about two standard deviations above normal) may proceed to have a carotid body modulation procedure. Following a carotid body modulation procedure the patient's chemosensitivity may optionally be tested again and compared to the results of the baseline test. The second test or the comparison of the second test to the baseline test may provide an indication of treatment success or suggest further intervention such as possible adjustment of drug therapy, repeating the carotid body modulation procedure with adjusted parameters or location, or performing another carotid body modulation procedure on a second carotid body if the first procedure only targeted one carotid body. It may be expected that a patient having chemoreceptor hypersensitivity or hyperactivity may return to about a normal sensitivity or activity following a successful carotid body modulation procedure.

In an alternative protocol for selecting a patient for a carotid body modulation, patients with high peripheral chemosensitivity or carotid body activity (e.g. ≧about 2 standard deviations above normal) alone or in combination with other clinical and physiologic parameters may be particularly good candidates for carotid body modulation therapy if they further respond positively to temporary blocking of carotid body activity. A prospective patient suffering from a cardiac, metabolic, or pulmonary disease may be selected to be tested to assess the baseline peripheral chemoreceptor sensitivity. A patient without high chemosensitivity may not be a plausible candidate for a carotid body modulation procedure. A patient with a high chemosensitivity may be given a further assessment that temporarily blocks a carotid body chemoreflex. For example a temporary block may be done chemically, for example using a chemical such as intravascular dopamine or dopamine-like substances, intravascular alpha-2 adrenergic agonists, oxygen, in general alkalinity, or local or topical application of atropine externally to the carotid body. A patient having a negative response to the temporary carotid body block test (e.g. sympathetic activity index such as respiration, HR, heart rate variability, MSNA, vasculature resistance, etc. is not significantly altered) may be a less plausible candidate for a carotid body modulation procedure. Conversely, a patient with a positive response to the temporary carotid body block test (e.g. respiration or index of sympathetic activity is altered significantly) may be a more plausible candidate for a carotid body modulation procedure.

There are a number of potential ways to conduct a temporary carotid body block test. Hyperoxia (e.g. higher than normal levels of PO₂) for example, is known to partially block (about a 50%) or reduce afferent sympathetic response of the carotid body. Thus, if a patient's sympathetic activity indexes (e.g. respiration, HR, HRV, MSNA) are reduced by hyperoxia (e.g. inhalation of higher than normal levels of O₂) for 3-5 minutes, the patient may be a particularly plausible candidate for carotid body modulation therapy. A sympathetic response to hyperoxia may be achieved by monitoring minute ventilation (e.g. reduction of more than 20-30% may indicate that a patient has carotid body hyperactivity). To evoke a carotid body response, or compare it to carotid body response in normoxic conditions, CO₂ above 3-4% may be mixed into the gas inspired by the patient (nitrogen content will be reduced) or another pharmacological agent can be used to invoke a carotid body response to a change of CO₂, pH or glucose concentration. Alternatively, “withdrawal of hypoxic drive” to rest state respiration in response to breathing a high concentration O₂ gas mix may be used for a simpler test.

An alternative temporary carotid body block test involves administering a sub-anesthetic amount of anesthetic gas halothane, which is known to temporarily suppress carotid body activity. Furthermore, there are injectable substances such as dopamine that are known to reversibly inhibit the carotid body. However, any substance, whether inhaled, injected or delivered by another manner to the carotid body that affects carotid body function in the desired fashion may be used.

Another alternative temporary carotid body block test involves application of cryogenic energy to a carotid body (i.e. removal of heat). For example, a carotid body or its nerves may be cooled to a temperature range between about −15° C. to 0° C. to temporarily reduce nerve activity or blood flow to and from a carotid body thus reducing or inhibiting carotid body activity.

An alternative method of assessing a temporary carotid body block test may involve measuring pulse pressure. Noninvasive pulse pressure devices such as Nexfin (made by BMEYE, based in Amsterdam, The Netherlands) can be used to track beat-to-beat changes in peripheral vascular resistance. Patients with hypertension or CHF may be sensitive to temporary carotid body blocking with oxygen or injection of a blocking drug. The peripheral vascular resistance of such patients may be expected to reduce substantially in response to carotid body blocking. Such patients may be good candidates for carotid body modulation therapy.

Yet another index that may be used to assess if a patient may be a good candidate for carotid body modulation therapy is increase of baroreflex, or baroreceptor sensitivity, in response to carotid body blocking. It is known that hyperactive chemosensitivity suppresses baroreflex. If carotid body activity is temporarily reduced the carotid sinus baroreflex (baroreflex sensitivity (BRS) or baroreflex gain) may be expected to increase. Baroreflex contributes a beneficial parasympathetic component to autonomic drive. Depressed BRS is often associated with an increased incidence of death and malignant ventricular arrhythmias. Baroreflex is measurable using standard non-invasive methods. One example is spectral analysis of RR interval of ECG and systolic blood pressure variability in both the high- and low-frequency bands. An increase of baroreflex gain in response to temporary blockade of carotid body can be a good indication for permanent therapy. Baroreflex sensitivity can also be measured by heart rate response to a transient rise in blood pressure induced by injection of phenylephrine.

An alternative method involves using an index of glucose tolerance to select patients and determine the results of carotid body blocking or removal in diabetic patients. There is evidence that carotid body hyperactivity contributes to progression and severity of metabolic disease.

In general, a beneficial response can be seen as an increase of parasympathetic or decrease of sympathetic tone in the overall autonomic balance. For example, Power Spectral Density (PSD) curves of respiration or HR can be calculated using nonparametric Fast Fourier Transform algorithm (FFT). FFT parameters can be set to 256-64 k buffer size, Hamming window, 50% overlap, 0 to 0.5 or 0.1 to 1.0 Hz range. HR and respiratory signals can be analyzed for the same periods of time corresponding to (1) normal unblocked carotid body breathing and (2) breathing with blocked carotid body.

Power can be calculated for three bands: the very low frequency (VLF) between 0 and 0.04 Hz, the low frequency band (LF) between 0.04-0.15 Hz and the high frequency band (HF) between 0.15-0.4 Hz. Cumulative spectral power in LF and HF bands may also be calculated; normalized to total power between 0.04 and 0.4 Hz (TF=HF+LF) and expressed as % of total. Natural breathing rate of CHF patient, for example, can be rather high, in the 0.3-0.4 Hz range.

The VLF band may be assumed to reflect periodic breathing frequency (typically 0.016 Hz) that can be present in CHF patients. It can be excluded from the HF/LF power ratio calculations.

The powers of the LF and HF oscillations characterizing heart rate variability (HRV) appear to reflect, in their reciprocal relationship, changes in the state of the sympathovagal (sympathetic to parasympathetic) balance occurring during numerous physiological and pathophysiological conditions. Thus, increase of HF contribution in particular can be considered a positive response to carotid body blocking.

Another alternative method of assessing carotid body activity comprises nuclear medicine scanning, for example with ocretide, somatostatin analogues, or other substances produced or bound by the carotid body.

Furthermore, artificially increasing blood flow may reduce carotid body activation. Conversely artificially reducing blood flow may stimulate carotid body activation. This may be achieved with drugs know in the art to alter blood flow.

There is a considerable amount of scientific evidence to demonstrate that hypertrophy of a carotid body often accompanies disease. A hypertrophied (i.e. enlarged) carotid body may further contribute to the disease. Thus identification of patients with enlarged carotid bodies may be instrumental in determining candidates for therapy. Imaging of a carotid body may be accomplished by angiography performed with radiographic, computer tomography, or magnetic resonance imaging.

It should be understood that the available measurements are not limited to those described above. It may be possible to use any single or a combination of measurements that reflect any clinical or physiological parameter effected or changed by either increases or decreases in carotid body function to evaluate the baseline state, or change in state, of a patient's chemosensitivity.

There is a considerable amount of scientific evidence to demonstrate that hypertrophy of a carotid body often accompanies disease. A hypertrophied or enlarged carotid body may further contribute to the disease. Thus identification of patients with enlarged carotid bodies may be instrumental in determining candidates for therapy.

Further, it is possible that although patients do not meet a preselected clinical or physiological definition of high peripheral chemosensitivity (e.g. greater than or equal to about two standard deviations above normal), administration of a substance that suppresses peripheral chemosensitivity may be an alternative method of identifying a patient who is a candidate for the proposed therapy. These patients may have a different physiology or co-morbid disease state that, in concert with a higher than normal peripheral chemosensitivity (e.g. greater than or equal to normal and less than or equal to about 2 standard deviations above normal), may still allow the patient to benefit from carotid body modulation. The proposed therapy may be at least in part based on an objective that carotid body modulation will result in a clinically significant or clinically beneficial change in the patient's physiological or clinical course. It is reasonable to believe that if the desired clinical or physiological changes occur even in the absence of meeting the predefined screening criteria, then therapy could be performed.

### Physiology:

Ablation of a target ablation site (e.g. peripheral chemoreceptor, carotid body) via an endovascular approach in patients having sympathetically mediated disease and augmented chemoreflex (e.g. high afferent nerve signaling from a carotid body to the central nervous system as in some cases indicated by high peripheral chemosensitivity) has been conceived to reduce peripheral chemosensitivity and reduce afferent signaling from peripheral chemoreceptors to the central nervous system. The expected reduction of chemoreflex activity and sensitivity to hypoxia and other stimuli such as blood flow, blood CO₂, glucose concentration or blood pH can directly reduce afferent signals from chemoreceptors and produce at least one beneficial effect such as the reduction of central sympathetic activation, reduction of the sensation of breathlessness (dyspnea), vasodilation, increase of exercise capacity, reduction of blood pressure, reduction of sodium and water retention, redistribution of blood volume to skeletal muscle, reduction of insulin resistance, reduction of hyperventilation, reduction of tachypnea, reduction of hypocapnia, increase of baroreflex and barosensitivity of baroreceptors, increase of vagal tone, or improve symptoms of a sympathetically mediated disease and may ultimately slow down the disease progression and extend life. It is understood that a sympathetically mediated disease that may be treated with carotid body modulation may comprise elevated sympathetic tone, an elevated sympathetic/parasympathetic activity ratio, autonomic imbalance primarily attributable to central sympathetic tone being abnormally or undesirably high, or heightened sympathetic tone at least partially attributable to afferent excitation traceable to hypersensitivity or hyperactivity of a peripheral chemoreceptor (e.g. carotid body). In some important clinical cases where baseline hypocapnia or tachypnea is present, reduction of hyperventilation and breathing rate may be expected. It is understood that hyperventilation in the context herein means respiration in excess of metabolic needs on the individual that generally leads to slight but significant hypocapnea (blood CO₂ partial pressure below normal of approximately 40 mmHg, for example in the range of 33 to 38 mmHg).

Patients having CHF or hypertension concurrent with heightened peripheral chemoreflex activity and sensitivity often react as if their system was hypercapnic even if it is not. The reaction is to hyperventilate, a maladaptive attempt to rid the system of CO₂, thus overcompensating and creating a hypocapnic and alkalotic system. Some researchers attribute this hypersensitivity/hyperactivity of the carotid body to the direct effect of catecholamines, hormones circulating in excessive quantities in the blood stream of CHF patients. The procedure may be particularly useful to treat such patients who are hypocapnic and possibly alkalotic resulting from high tonic output from carotid bodies. Such patients are particularly predisposed to periodic breathing and central apnea hypopnea type events that cause arousal, disrupt sleep, cause intermittent hypoxia and are by themselves detrimental and difficult to treat.

It is appreciated that periodic breathing of Cheyne Stokes pattern occurs in patients during sleep, exercise and even at rest as a combination of central hypersensitivity to CO₂, peripheral chemosensitivity to O₂ and CO₂ and prolonged circulatory delay. All these parameters are often present in CHF patients that are at high risk of death. Thus, patients with hypocapnea, CHF, high chemosensitivity and prolonged circulatory delay, and specifically ones that exhibit periodic breathing at rest or during exercise or induced by hypoxia are likely beneficiaries of the proposed therapy.

Hyperventilation is defined as breathing in excess of a person's metabolic need at a given time and level of activity. Hyperventilation is more specifically defined as minute ventilation in excess of that needed to remove CO2 from blood in order to maintain blood CO₂ in the normal range (e.g. around 40 mmHg partial pressure). For example, patients with arterial blood PCO₂ in the range of 32-37 mmHg can be considered hypocapnic and in hyperventilation.

For the purpose of this disclosure hyperventilation is equivalent to abnormally low levels of carbon dioxide in the blood (e.g. hypocapnia, hypocapnea, or hypocarbia) caused by overbreathing. Hyperventilation is the opposite of hypoventilation (e.g. underventilation) that often occurs in patients with lung disease and results in high levels of carbon dioxide in the blood (e.g. hypercapnia or hypercarbia).

A low partial pressure of carbon dioxide in the blood causes alkalosis, because CO2 is acidic in solution and reduced CO2 makes blood pH more basic, leading to lowered plasma calcium ions and nerve and muscle excitability. This condition is undesirable in cardiac patients since it can increase probability of cardiac arrhythmias.

Alkalemia may be defined as abnormal alkalinity, or increased pH of the blood. Respiratory alkalosis is a state due to excess loss of carbon dioxide from the body, usually as a result of hyperventilation. Compensated alkalosis is a form in which compensatory mechanisms have returned the pH toward normal. For example, compensation can be achieved by increased excretion of bicarbonate by the kidneys.

Compensated alkalosis at rest can become uncompensated during exercise or as a result of other changes of metabolic balance. Thus the invented method is applicable to treatment of both uncompensated and compensated respiratory alkalosis.

Tachypnea means rapid breathing. For the purpose of this disclosure a breathing rate of about 6 to 16 breaths per minute at rest is considered normal but there is a known benefit to lower rate of breathing in cardiac patients. Reduction of tachypnea can be expected to reduce respiratory dead space, increase breathing efficiency, and increase parasympathetic tone.

Therapy Example: Role of Chemoreflex and Central Sympathetic Nerve Activity in CHF

Chronic elevation in sympathetic nerve activity (SNA) is associated with the development and progression of certain types of hypertension and contributes to the progression of congestive heart failure (CHF). It is also known that sympathetic excitatory cardiac, somatic, and central/peripheral chemoreceptor reflexes are abnormally enhanced in CHF and hypertension (Ponikowski, 2011 and Giannoni, 2008 and 2009).

Arterial chemoreceptors serve an important regulatory role in the control of alveolar ventilation. They also exert a powerful influence on cardiovascular function.

Delivery of Oxygen (O₂) and removal of Carbon Dioxide (CO₂) in the human body is regulated by two control systems, behavioral control and metabolic control. The metabolic ventilatory control system drives our breathing at rest and ensures optimal cellular homeostasis with respect to pH, partial pressure of carbon dioxide (PCO₂), and partial pressure of oxygen (PO₂). Metabolic control uses two sets of chemoreceptors that provide a fine-tuning function: the central chemoreceptors located in the ventral medulla of the brain and the peripheral chemoreceptors such as the aortic chemoreceptors and the carotid body chemoreceptors. The carotid body, a small, ovoid-shaped (often described as a grain of rice), and highly vascularized organ is situated in or near the carotid bifurcation, where the common carotid artery branches in to an internal carotid artery (IC) and external carotid artery (EC). The central chemoreceptors are sensitive to hypercapnia (high PCO₂), and the peripheral chemoreceptors are sensitive to hypercapnia and hypoxia (low blood PO₂). Under normal conditions activation of the sensors by their respective stimuli results in quick ventilatory responses aimed at the restoration of cellular homeostasis.

As early as 1868, Pflüger recognized that hypoxia stimulated ventilation, which spurred a search for the location of oxygen-sensitive receptors both within the brain and at various sites in the peripheral circulation. When Corneille Heymans and his colleagues observed that ventilation increased when the oxygen content of the blood flowing through the bifurcation of the common carotid artery was reduced (winning him the Nobel Prize in 1938), the search for the oxygen chemosensor responsible for the ventilatory response to hypoxia was largely considered accomplished.

The persistence of stimulatory effects of hypoxia in the absence (after surgical removal) of the carotid chemoreceptors (e.g. the carotid bodies) led other investigators, among them Julius Comroe, to ascribe hypoxic chemosensitivity to other sites, including both peripheral sites (e.g., aortic bodies) and central brain sites (e.g., hypothalamus, pons and rostral ventrolateral medulla). The aortic chemoreceptor, located in the aortic body, may also be an important chemoreceptor in humans with significant influence on vascular tone and cardiac function.

Carotid Body Chemoreflex:

The carotid body is a small cluster of chemoreceptors (also known as glomus cells) and supporting cells located near, and in most cases directly at, the medial side of the bifurcation (fork) of the carotid artery, which runs along both sides of the throat.

These organs act as sensors detecting different chemical stimuli from arterial blood and triggering an action potential in the afferent fibers that communicate this information to the Central Nervous System (CNS). In response, the CNS activates reflexes that control heart rate (HR), renal function and peripheral blood circulation to maintain the desired homeostasis of blood gases, O₂ and CO₂, and blood pH. This closed loop control function that involves blood gas chemoreceptors is known as the carotid body chemoreflex (CBC). The carotid body chemoreflex is integrated in the CNS with the carotid sinus baroreflex (CSB) that maintains arterial blood pressure. In a healthy organism these two reflexes maintain blood pressure and blood gases within a narrow physiologic range. Chemosensors and barosensors in the aortic arch contribute redundancy and fine-tuning function to the closed loop chemoreflex and baroreflex. In addition to sensing blood gasses, the carotid body is now understood to be sensitive to blood flow and velocity, blood Ph and glucose concentration. Thus it is understood that in conditions such as hypertension, CHF, insulin resistance, diabetes and other metabolic derangements afferent signaling of carotid body nerves may be elevated. Carotid body hyperactivity may be present even in the absence of detectable hypersensitivity to hypoxia and hypercapnia that are traditionally used to index carotid body function. The purpose of the proposed therapy is therefore to remove or reduce afferent neural signals from a carotid body and reduce carotid body contribution to central sympathetic tone.

The carotid sinus baroreflex is accomplished by negative feedback systems incorporating pressure sensors (e.g., baroreceptors) that sense the arterial pressure. Baroreceptors also exist in other places, such as the aorta and coronary arteries. Important arterial baroreceptors are located in the carotid sinus, a slight dilatation of the internal carotid artery at its origin from the common carotid. The carotid sinus baroreceptors are close to but anatomically separate from the carotid body. Baroreceptors respond to stretching of the arterial wall and communicate blood pressure information to CNS. Baroreceptors are distributed in the arterial walls of the carotid sinus while the chemoreceptors (glomus cells) are clustered inside the carotid body. This makes the selective reduction of chemoreflex described in this application possible while substantially sparing the baroreflex.

The carotid body exhibits great sensitivity to hypoxia (low threshold and high gain). In chronic Congestive Heart Failure (CHF), the sympathetic nervous system activation that is directed to attenuate systemic hypoperfusion at the initial phases of CHF may ultimately exacerbate the progression of cardiac dysfunction that subsequently increases the extra-cardiac abnormalities, a positive feedback cycle of progressive deterioration, a vicious cycle with ominous consequences. It was thought that much of the increase in the sympathetic nerve activity (SNA) in CHF was based on an increase of sympathetic flow at a level of the CNS and on the depression of arterial baroreflex function. In the past several years, it has been demonstrated that an increase in the activity and sensitivity of peripheral chemoreceptors (heightened chemoreflex function) also plays an important role in the enhanced SNA that occurs in CHF.

Role of Altered Chemoreflex in CHF:

As often happens in chronic disease states, chemoreflexes that are dedicated under normal conditions to maintaining homeostasis and correcting hypoxia contribute to increase the sympathetic tone in patients with CHF, even under normoxic conditions. The understanding of how abnormally enhanced sensitivity of the peripheral chemosensors, particularly the carotid body, contributes to the tonic elevation in SNA in patients with CHF has come from several studies in animals. According to one theory, the local angiotensin receptor system plays a fundamental role in the enhanced carotid body chemoreceptor sensitivity in CHF. In addition, evidence in both CHF patients and animal models of CHF has clearly established that the carotid body chemoreflex is often hypersensitive in CHF patients and contributes to the tonic elevation in sympathetic function. This derangement derives from altered function at the level of both the afferent and central pathways of the reflex arc. The mechanisms responsible for elevated afferent activity from the carotid body in CHF are not yet fully understood.

Regardless of the exact mechanism behind the carotid body hypersensitivity, the chronic sympathetic activation driven from the carotid body and other autonomic pathways leads to further deterioration of cardiac function in a positive feedback cycle. As CHF ensues, the increasing severity of cardiac dysfunction leads to progressive escalation of these alterations in carotid body chemoreflex function to further elevate sympathetic activity and cardiac deterioration. The trigger or causative factors that occur in the development of CHF that sets this cascade of events in motion and the time course over which they occur remain obscure. Ultimately, however, causative factors are tied to the cardiac pump failure and reduced cardiac output. According to one theory, within the carotid body, a progressive and chronic reduction in blood flow may be the key to initiating the maladaptive changes that occur in carotid body chemoreflex function in CHF.

There is sufficient evidence that there is increased peripheral and central chemoreflex sensitivity in heart failure, which is likely to be correlated with the severity of the disease. There is also some evidence that the central chemoreflex is modulated by the peripheral chemoreflex. According to current theories, the carotid body is the predominant contributor to the peripheral chemoreflex in humans; the aortic body having a minor contribution.

Although the mechanisms responsible for altered central chemoreflex sensitivity remain obscure, the enhanced peripheral chemoreflex sensitivity can be linked to a depression of nitric oxide production in the carotid body affecting afferent sensitivity, and an elevation of central angiotensin II affecting central integration of chemoreceptor input. The enhanced chemoreflex may be responsible, in part, for the enhanced ventilatory response to exercise, dyspnea, Cheyne-Stokes breathing, and sympathetic activation observed in chronic heart failure patients. The enhanced chemoreflex may be also responsible for hyperventilation and tachypnea (e.g. fast breathing) at rest and exercise, periodic breathing during exercise, rest and sleep, hypocapnia, vasoconstriction, reduced peripheral organ perfusion and hypertension.

Dyspnea:

Shortness of breath, or dyspnea, is a feeling of difficult or labored breathing that is out of proportion to the patient's level of physical activity. It is a symptom of a variety of different diseases or disorders and may be either acute or chronic. Dyspnea is the most common complaint of patients with cardiopulmonary diseases.

Dyspnea is believed to result from complex interactions between neural signaling, the mechanics of breathing, and the related response of the central nervous system. A specific area has been identified in the mid-brain that may influence the perception of breathing difficulties.

The experience of dyspnea depends on its severity and underlying causes. The feeling itself results from a combination of impulses relayed to the brain from nerve endings in the lungs, rib cage, chest muscles, or diaphragm, combined with the perception and interpretation of the sensation by the patient. In some cases, the patient's sensation of breathlessness is intensified by anxiety about its cause. Patients describe dyspnea variously as unpleasant shortness of breath, a feeling of increased effort or tiredness in moving the chest muscles, a panicky feeling of being smothered, or a sense of tightness or cramping in the chest wall.

The four generally accepted categories of dyspnea are based on its causes: cardiac, pulmonary, mixed cardiac or pulmonary, and non-cardiac or non-pulmonary. The most common heart and lung diseases that produce dyspnea are asthma, pneumonia, COPD, and myocardial ischemia or heart attack (myocardial infarction). Foreign body inhalation, toxic damage to the airway, pulmonary embolism, congestive heart failure (CHF), anxiety with hyperventilation (panic disorder), anemia, and physical deconditioning because of sedentary lifestyle or obesity can produce dyspnea. In most cases, dyspnea occurs with exacerbation of the underlying disease. Dyspnea also can result from weakness or injury to the chest wall or chest muscles, decreased lung elasticity, obstruction of the airway, increased oxygen demand, or poor pumping action of the heart that results in increased pressure and fluid in the lungs, such as in CHF.

Acute dyspnea with sudden onset is a frequent cause of emergency room visits. Most cases of acute dyspnea involve pulmonary (lung and breathing) disorders, cardiovascular disease, or chest trauma. Sudden onset of dyspnea (acute dyspnea) is most typically associated with narrowing of the airways or airflow obstruction (bronchospasm), blockage of one of the arteries of the lung (pulmonary embolism), acute heart failure or myocardial infarction, pneumonia, or panic disorder.

Chronic dyspnea is different. Long-standing dyspnea (chronic dyspnea) is most often a manifestation of chronic or progressive diseases of the lung or heart, such as COPD, which includes chronic bronchitis and emphysema. The treatment of chronic dyspnea depends on the underlying disorder. Asthma can often be managed with a combination of medications to reduce airway spasms and removal of allergens from the patient's environment. COPD requires medication, lifestyle changes, and long-term physical rehabilitation. Anxiety disorders are usually treated with a combination of medication and psychotherapy.

Although the exact mechanism of dyspnea in different disease states is debated, there is no doubt that the CBC plays some role in most manifestations of this symptom. Dyspnea seems to occur most commonly when afferent input from peripheral receptors is enhanced or when cortical perception of respiratory work is excessive.

Surgical Removal of the Glomus and Resection of Carotid Body Nerves:

A surgical treatment for asthma, removal of the carotid body or glomus (glomectomy), was described by Japanese surgeon Komei Nakayama in 1940s. According to Nakayama in his study of 4,000 patients with asthma, approximately 80% were cured or improved six months after surgery and 58% allegedly maintained good results after five years. Komei Nakayama performed most of his surgeries while at the Chiba University during World War II. Later in the 1950's, a U.S. surgeon, Dr. Overholt, performed the Nakayama operation on 160 U.S. patients. He felt it necessary to remove both carotid bodies in only three cases. He reported that some patients feel relief the instant when the carotid body is removed, or even earlier, when it is inactivated by an injection of procaine (Novocain).

Overholt, in his paper Glomectomy for Asthma published in Chest in 1961, described surgical glomectomy the following way: “A two-inch incision is placed in a crease line in the neck, one-third of the distance between the angle of the mandible and clavicle. The platysma muscle is divided and the sternocleidomastoid retracted laterally. The dissection is carried down to the carotid sheath exposing the bifurcation. The superior thyroid artery is ligated and divided near its take-off in order to facilitate rotation of the carotid bulb and expose the medial aspect of the bifurcation. The carotid body is about the size of a grain of rice and is hidden within the adventitia of the vessel and is of the same color. The perivascular adventitia is removed from one centimeter above to one centimeter below the bifurcation. This severs connections of the nerve plexus, which surrounds the carotid body. The dissection of the adventitia is necessary in order to locate and identify the body. It is usually located exactly at the point of bifurcation on its medial aspect. Rarely, it may be found either in the center of the crotch or on the lateral wall. The small artery entering the carotid body is clamped, divided, and ligated. The upper stalk of tissue above the carotid body is then clamped, divided, and ligated.”

In January 1965, the New England Journal of Medicine published a report of 15 cases in which there had been unilateral removal of the cervical glomus (carotid body) for the treatment of bronchial asthma, with no objective beneficial effect. This effectively stopped the practice of glomectomy to treat asthma in the U.S.

Winter developed a technique for separating nerves that contribute to the carotid sinus nerves into two bundles, carotid sinus (baroreflex) and carotid body (chemoreflex), and selectively cutting out the latter. The Winter technique is based on his discovery that carotid sinus (baroreflex) nerves are predominantly on the lateral side of the carotid bifurcation and carotid body (chemoreflex) nerves are predominantly on the medial side.

Neuromodulation of the Carotid Body Chemoreflex:

Hlavaka in U.S. Patent Application Publication 2010/0070004 filed Aug. 7, 2009, describes implanting an electrical stimulator to apply electrical signals, which block or inhibit chemoreceptor signals in a patient suffering dyspnea. Hlavaka teaches that “some patients may benefit from the ability to reactivate or modulate chemoreceptor functioning.” Hlavaka focuses on neuromodulation of the chemoreflex by selectively blocking conduction of nerves that connect the carotid body to the CNS. Hlavaka describes a traditional approach of neuromodulation with an implantable electric pulse generator that does not modify or alter tissue of the carotid body or chemoreceptors.

The central chemoreceptors are located in the brain and are difficult to access. The peripheral chemoreflex is modulated primarily by carotid bodies that are more accessible. Previous clinical practice had very limited clinical success with the surgical removal of carotid bodies to treat asthma in 1940s and 1960s.

While the invention has been described in connection with what is presently considered to be the best mode, it is to be understood that the invention is not to be limited to the disclosed embodiment(s). The invention covers various modifications and equivalent arrangements included within the spirit and scope of the appended claims.

We claim:

1. A vascular catheter configured for ablation of perivascular tissue comprising:
a. a flexible elongated structure comprising a distal end and a proximal end;
b. a hollow cylindrical structure located in the vicinity of the distal end comprising an electrically non-conductive outer surface, an inner surface that is at least in part electrically conductive, and at least one lateral fenestration;
c. at least one channel in fluidic communication between the interior of the hollow cylindrical structure and a fluid connector in the vicinity of the proximal end; and,
d. at least one wire in electrical communication between the inner electrical conductive surface and an electrical connector in the vicinity of the proximal end.

2. The vascular catheter of claim 1 further comprising a temperature sensor mounted in the vicinity of the at least one lateral fenestration configured for measuring a vascular tissue temperature.

3. The vascular catheter of claim 1 or 2 further comprising a mechanism for pressing the at least one lateral fenestration against the inner wall of a blood vessel while providing a substantially unambiguous fluoroscopic indication of the position of the lateral fenestration within said blood vessel.

4. The vascular catheter of claim 3 wherein the mechanism comprises at least one retractable radiopaque wire loop located in substantially diametric opposition to the at least one lateral fenestration in the vicinity of the distal end.

5. The vascular catheter of claim 3 wherein the mechanism comprises an inflatable structure in substantially diametric opposition to the at least one lateral fenestration in the vicinity of the distal end.

6. The vascular catheter of claim 3 wherein the mechanism comprises a pull wire within the flexible elongated structure between the hollow cylindrical structure and an actuator located in the vicinity of the proximal end.

7. The vascular catheter of claims 1 to 6 further comprises a lumen configured for use with a guidewire distal to the hollow cylindrical structure.

8. The vascular catheter of claim 7 wherein the proximal terminal of the lumen configured for use with a guidewire is distal to the hollow cylindrical structure.

9. The vascular catheter of claim 7 wherein the proximal terminal of the lumen configured for use with a guidewire is proximal to the hollow cylindrical structure.

10. The vascular catheter of any claims 1 to 9 wherein the flexible elongated structure is a thermoplastic material fabricated using an extrusion process.

11. The vascular catheter of claim 10 wherein the elongated structure comprises a woven, coiled, or knitted structure configured for torsional rigidity along the length of the structure.

12. The vascular catheter of any claims 1 to 11 wherein the hollow cylindrical structure is a composite structure comprising a machined metallic tubular structure with and an applied electrically insulative outer layer.

13. The vascular catheter of any claims 1 to 11 wherein the hollow cylindrical structure is a composite structure comprising a non-metallic tubular structure with a metallic material applied to at least a portion of the inner surface.

14. A vascular catheter configured for ablation of perivascular tissue comprising:
a. a flexible elongated structure comprising a distal and, and a proximal end;
b. a hollow cylindrical structure that is substantially not electrically conductive located in the vicinity of the distal end comprising at least one lateral fenestration;
c. at least one electrode mounted within the hollow cylindrical structure;
d. at least one channel in fluidic communication between the interior of the hollow cylindrical structure and a fluid connector in the vicinity of the proximal end; and,
e. at least one wire in electrical communication between the electrode and an electrical connector in the vicinity of the proximal end.

15. The vascular catheter of claim 14 further comprising a temperature sensor mounted in the vicinity of the at least one lateral fenestration configured for measuring a vascular tissue temperature.

16. The vascular catheter of claim 14 or 15 further comprising a mechanism for pressing the at least one lateral fenestration against the inner wall of a blood vessel while providing a substantially unambiguous fluoroscopic indication of the position of the lateral fenestration within said blood vessel.

17. The vascular catheter of claim 16 wherein the mechanism comprises at least one retractable radiopaque wire loop located in substantially diametric opposition to the at least one lateral fenestration in the vicinity of the distal end.

18. The vascular catheter of claim 16 wherein the mechanism comprises an inflatable structure in substantially diametric opposition to the at least one lateral fenestration in the vicinity of the distal end.

19. The vascular catheter of claim 16 wherein the mechanism comprises a pull wire within the flexible elongated structure between the hollow cylindrical structure and an actuator located in the vicinity of the proximal end.

20. The vascular catheter of claims 15 to 19 further comprises a lumen configured for use with a guidewire distal to the hollow cylindrical structure.

21. The vascular catheter of claim 20 wherein the proximal terminal of the lumen configured for use with a guidewire is distal to the hollow cylindrical structure.

22. The vascular catheter of claim 20 wherein the proximal terminal of the lumen configured for use with a guidewire is proximal to the hollow cylindrical structure.

23. The vascular catheter of any claims 15 to 22 wherein the flexible elongated structure is a thermoplastic material fabricated using an extrusion process.

24. The vascular catheter of claim 23 wherein the elongated structure comprises a woven, coiled, or knitted structure configured for torsional rigidity along the length of the structure.

25. A vascular catheter configured for ablation of perivascular tissue comprising:
a. a flexible elongated structure comprising a distal and, and a proximal end;
b. an inflatable structure that is substantially not electrically conductive located in the vicinity of the distal end comprising at least one lateral fenestration;
c. at least one electrode mounted within the inflatable structure;
d. at least one channel in fluidic communication between the interior of the inflatable structure and a fluid connector in the vicinity of the proximal end; and,
e. at least one wire in electrical communication between the electrode and an electrical connector in the vicinity of the proximal end.

26. The vascular catheter of claim 25 further comprising a temperature sensor mounted in the vicinity of the at least one lateral fenestration configured for measuring a vascular tissue temperature.

27. The vascular catheter of claim 25 or 26 further comprising a mechanism for pressing the at least one lateral fenestration against the inner wall of a blood vessel while providing a substantially unambiguous fluoroscopic indication of the position of the lateral fenestration within said blood vessel.

28. The vascular catheter of claim 27 wherein the mechanism comprises at least one retractable radiopaque wire loop located in substantially diametric opposition to the at least one lateral fenestration in the vicinity of the distal end.

29. The vascular catheter of claim 27 wherein the mechanism comprises an inflatable structure in substantially diametric opposition to the at least one lateral fenestration in the vicinity of the distal end.

30. The vascular catheter of claim 27 wherein the mechanism comprises a pull wire within the flexible elongated structure between the hollow cylindrical structure and an actuator located in the vicinity of the proximal end.

31. The vascular catheter of claims 25 to 30 further comprises a lumen configured for use with a guidewire distal to the hollow cylindrical structure.

32. The vascular catheter of claim 31 wherein the proximal terminal of the lumen configured for use with a guidewire is distal to the hollow cylindrical structure.

33. The vascular catheter of claim 31 wherein the proximal terminal of the lumen configured for use with a guidewire is proximal to the hollow cylindrical structure.

34. The vascular catheter of any claims 25 to 33 wherein the flexible elongated structure is a thermoplastic material fabricated using an extrusion process.

35. The vascular catheter of claim 34 wherein the elongated structure comprises a woven, coiled, or knitted structure configured for torsional rigidity along the length of the structure.

36. The vascular catheter of any claims 25 to 35 wherein the inflatable structure(s) comprises an elastomeric balloon.

37. The vascular catheter of any claims 25 to 36 wherein the inflatable structure(s) comprises non-elastomeric balloon.

38. A vascular catheter configured for ablation of perivascular tissue comprising:
a. a flexible elongated structure comprising a distal and, and a proximal end;
b. a forceps mechanism mounted in the vicinity of the distal end comprising at least one hollow cylindrical structure comprising an electrically insulated outer surface, an interior electrode, and at least on lateral fenestration oriented in the direction of the opposing forceps element;
c. at least one channel in fluidic communication between the interior of the hollow cylindrical structure and a fluid connector in the vicinity of the proximal end; and,
d. at least one wire in electrical communication between the interior electrode and an electrical connector in the vicinity of the proximal end.

39. The vascular catheter of claim 38 wherein opposing forceps element comprises a forceps arm with an inflatable structure mounted in the vicinity of the distal end.

40. The vascular catheter of claim 39 wherein the inflatable structure comprises an interior electrode, at least one fluid channel between the interior of the inflatable structure and a fluid connector in the vicinity of the proximal end, at least one wire in electrical communication between the interior electrode and an electrical connector in the vicinity of the proximal end, and at least one lateral fenestration in the wall of the inflatable structure.

41. The vascular catheter of claim 40 wherein the at least one lateral fenestration is oriented in the direction of the opposing forceps element.

42. The vascular catheter of any claims 39 to 41 wherein the forceps arm and inflatable structure substantially reside within the hollow cylindrical structure prior to deployment.

43. The vascular catheter of any claims 39 to 42 wherein the forceps arm and inflatable structure are configured for retraction into the hollow cylindrical structure.

44. The vascular catheter of claims 42 and 43 wherein the deployment or retraction is facilitated by a slidable outer sheath.

45. A vascular catheter configured for ablation of perivascular tissue comprising:
a. a flexible elongated structure comprising a distal and, and a proximal end;
b. a forceps mechanism mounted in the vicinity of the distal end comprising two forceps arms;
c. an inflatable structure mounted in the vicinity of the distal end of each arm;
d. at least one electrode mounted within each inflatable structure;
e. at least one fluid channel in communication between the interior of each inflatable structure and a fluid connector in the vicinity of the proximal end;
f. at least one wire in electrical communication between each electrode and an electrical connector in the vicinity of the proximal end; and,
g. at least one fenestration in each inflatable structure oriented in the direction of the opposing forceps element.

46. The vascular catheter of claim 45 wherein the electrical connector is configured for bipolar RF ablation.

47. The vascular catheter of claim 45 wherein the electrical connector is configured for monopolar RF ablation.

48. The vascular catheter of claim 45 wherein the forceps mechanism is facilitated by means of a slidable outer sheath.

49. The vascular catheter of claim 45 wherein the forceps mechanism is facilitated by means of inflation of the inflatable structures.

50. A vascular catheter configured for ablation of perivascular tissue comprising:
a. a flexible elongated structure comprising a distal and, and a proximal end;
b. a forceps mechanism mounted in the vicinity of the distal end comprising two forceps arms;
c. a substantially non-electrically conductive porous structure mounted in the vicinity of the distal end of each arm;
d. at least one electrode mounted in contact with each porous structure;
e. at least one fluid channel in communication between each porous structure and a fluid connector in the vicinity of the proximal end; and,
f. at least one wire in electrical communication between each electrode and an electrical connector in the vicinity of the proximal end.

51. The vascular catheter of claim 50 wherein the porous structure comprises an open cell elastomeric foam.

52. The vascular catheter of claim 51 wherein the open cell elastomeric foam comprises silicone rubber

53. The vascular catheter of any claims 50 to 52 wherein the at electrical connector is configured for bipolar RF ablation.

54. The vascular catheter of any claims 50 to 52 wherein the electrical connector is configured for monopolar RF ablation.

55. The vascular catheter of any claims 50 to 54 wherein the forceps mechanism is facilitated by means of a slidable outer sheath.

56. A vascular catheter configured for ablation of perivascular tissue comprising:
a. a flexible elongated structure comprising a distal and, and a proximal end;
b. a hollow cylindrical structure that is substantially not electrically conductive located in the vicinity of the distal end comprising at least one lateral fenestration;
c. at least one electrode mounted within the hollow cylindrical structure;
d. at least one channel in fluidic communication between the interior of the hollow cylindrical structure and a fluid connector in the vicinity of the proximal end;
e. at least one wire in electrical communication between the electrode and an electrical connector in the vicinity of the proximal end; and,
f. an elastomeric membrane comprising a slit covering the at least one lateral fenestration;
whereby, the slit functions as a one-way fluid valve.

57. A vascular catheter configured for ablation of perivascular tissue comprising:
a. a flexible elongated structure comprising a distal and, and a proximal end;
b. a hollow cylindrical structure located in the vicinity of the distal end comprising at least one lateral fenestration;
c. at least one Piezo-electric element mounted within the hollow cylindrical structure configured for directed ultrasonic emission through the at least one lateral fenestration;
d. at least one channel in fluidic communication between the interior of the hollow cylindrical structure and a fluid connector in the vicinity of the proximal end;
e. at least one coaxial cable in electrical communication between the Piezo-electric element and an electrical connector in the vicinity of the proximal end.

58. A vascular catheter configured for ablation of perivascular tissue comprising:
a. a flexible elongated structure comprising a distal and, and a proximal end;
b. a hollow cylindrical structure located in the vicinity of the distal end comprising at least one lateral fenestration;
c. at least one Piezo-electric element mounted within the hollow cylindrical structure configured for directed ultrasonic emission through the at least one lateral fenestration;
d. at least one electrode element mounted within the hollow cylindrical structure;
e. at least one channel in fluidic communication between the interior of the hollow cylindrical structure and a fluid connector in the vicinity of the proximal end;
f. at least one coaxial cable in electrical communication between the Piezo-electric element and an electrical connector in the vicinity of the proximal end; and
g. electrical communication between the electrode and an electrical connector in the vicinity of the distal end.

59. A method for ablating perivascular tissue of a patient comprising:
a. inserting an ablation device into a blood vessel of the patient, said ablation device comprising an elongated structure with a distal end and a proximal end, a hollow cylindrical structure located in the vicinity of the distal end comprising an electrically insulated outer surface and at least one lateral fenestration, at least one electrode mounted within the hollow cylindrical structure, at least one channel in fluidic communication between the interior of the hollow cylindrical structure and fluid connector in the vicinity of the proximal end, at least one wire in electrical communication between the at least one electrode and an electrical connector in the vicinity of the proximal end, and a mechanism for pressing the lateral fenestration against the wall of a blood vessel;
b. connecting the ablation device to a source of RF ablation energy, and a source of ionic liquid;
c. advancing the distal end of the ablation device proximate to the perivascular ablation target;
d. pressing the lateral fenestration against the wall of the blood vessel oriented towards the perivascular ablation target; then,
e. delivering an ionic liquid to the hollow cylindrical structure in a substantially continuous manner; then,
f. applying RF energy to the electrode at a level and duration sufficient for ablation of the target perivascular tissue;
whereby, the ionic liquid substantially displaces blood from the space between the vascular wall and the electrode, while conducting electrical energy between the vascular wall and the electrode through the vascular wall surface defined by the fenestration.

60. A method for ablating perivascular tissue of a patient comprising:
a. inserting an ablation device into a blood vessel of the patient, said ablation device comprising an elongated structure with a distal end and a proximal end, an inflatable structure that is substantially not electrically conductive located in the vicinity of the distal end comprising at least one lateral fenestration, at least one electrode mounted within the inflatable structure, at least one channel in fluidic communication between the interior of the inflatable structure and a fluid connector in the vicinity of the proximal end and, at least one wire in electrical communication between the electrode and an electrical connector in the vicinity of the proximal end, and a mechanism for pressing the lateral fenestration against the wall of a blood vessel;
b. connecting the ablation device to a source of RF ablation energy, and a source of ionic liquid;
c. advancing the distal end of the ablation device through the vasculature of the patient proximate to a target perivascular tissue;
d. delivering an ionic liquid to the inflatable structure in a substantially continuous manner; then,
e. pressing the lateral fenestration against the wall of the blood vessel oriented towards the target perivascular tissue; then,
f. applying RF energy to the electrode at a level and duration sufficient for ablation of the target perivascular tissue;
whereby, the ionic liquid inflates the inflatable structure, substantially displaces blood from the space between the vascular wall and the at least one electrode, while conducting electrical energy between the vascular wall and the at least one electrode through the vascular wall surface defined by the at least one fenestration.

61. A method for ablating carotid body function in a patient comprising:
a. inserting an ablation device into a peripheral artery of the patient, said ablation device comprising an elongated structure with a distal end and a proximal end, a forceps mechanism mounted in the vicinity of the distal end comprising at least one hollow cylindrical structure comprising an electrically insulated outer surface, an interior electrode, and at least on lateral fenestration oriented in the direction of the opposing forceps element, at least one channel in fluidic communication between the interior of the hollow cylindrical structure and a fluid connector in the vicinity of the proximal end, at least one wire in electrical communication between the interior electrode and an electrical connector in the vicinity of the proximal end;
b. connecting the ablation device to a source of RF ablation energy, and a source of ionic liquid;
c. advancing the distal end of the ablation device through the arterial system of the patient proximate to a carotid bifurcation associated with the target carotid body; then,
d. deploying the forceps mechanism and grasping the carotid bifurcation saddle; then,
e. delivering an ionic liquid to the hollow cylindrical structure in a substantially continuous manner; then,
f. applying RF energy to the electrode at a level and duration sufficient for ablation of the target perivascular tissue;
whereby, the ionic liquid substantially displaces blood from the space between the vascular wall and the at least one electrode, while conducting electrical energy between the vascular wall and the at least one electrode through the vascular wall surface defined by the at least one fenestration.

62. A method for ablating carotid body function in a patient comprising:
a. inserting an ablation device into a peripheral artery of the patient, said ablation device comprising an elongated structure with a distal end and a proximal end, a forceps mechanism mounted in the vicinity of the distal end comprising two forceps arms, an inflatable structure mounted in the vicinity of the distal end of each arm, at least one electrode mounted within each inflatable structure, at least one fluid channel in communication between the interior of each inflatable structure and a fluid connector in the vicinity of the proximal end, at least one wire in electrical communication between each electrode and an electrical connector in the vicinity of the proximal end and, at least one fenestration in each inflatable structure oriented in the direction of the opposing forceps element;
b. connecting the ablation device to a source of RF ablation energy, and a source of ionic liquid;
c. advancing the distal end of the ablation device through the arterial system of the patient proximate to a carotid bifurcation associated with the target carotid body; then,
d. deploying the forceps mechanism and grasping the carotid bifurcation saddle; then,
e. delivering an ionic liquid to the inflatable structures in a substantially continuous manner; then,
f. applying RF energy to the electrode at a level and duration sufficient for ablation of the target perivascular tissue;
whereby, the ionic liquid substantially inflates the inflatable structures, displaces blood from the space between the vascular wall and the at least one electrode within each inflatable structure, while conducting electrical energy between the vascular wall and the at least one electrode through the vascular wall surface defined by the at least one fenestration.

63. A method for ablating carotid body function in a patient comprising:
a. inserting an ablation device into a peripheral artery of the patient, said ablation device comprising an elongated structure with a distal end and a proximal end, a forceps mechanism mounted in the vicinity of the distal end comprising two forceps arms, a substantially non-electrically conductive porous structure mounted in the vicinity of the distal end of each arm, at least one electrode mounted in contact with each porous structure, at least one fluid channel in communication between each porous structure and a fluid connector in the vicinity of the proximal end and, at least one wire in electrical communication between each electrode and an electrical connector in the vicinity of the proximal end;
b. advancing the distal end of the ablation device through the arterial system of the patient proximate to a carotid bifurcation associated with the target carotid body; then,
c. deploying the forceps mechanism and grasping the carotid bifurcation saddle; then,
d. delivering an ionic liquid to the inflatable structures in a substantially continuous manner; then,
e. applying RF energy to the electrode at a level and duration sufficient for ablation of the target perivascular tissue;
whereby, the ionic liquid substantially inflates the inflatable structures, displaces blood from the space between the vascular wall and the at least one electrode within each inflatable structure, while conducting electrical energy between the vascular wall and the at least one electrode through the vascular wall surface defined by the at least one fenestration.

64. A method for ablating perivascular tissue of a patient comprising:
a. inserting an ablation device into a blood vessel of the patient, said ablation device comprising an elongated structure with a distal end and a proximal end, a hollow cylindrical structure located in the vicinity of the distal end comprising at least one lateral fenestration, at least one Piezo-electric element mounted within the hollow cylindrical structure configured for directed ultrasonic emission through the at least one lateral fenestration, at least one channel in fluidic communication between the interior of the hollow cylindrical structure and a fluid connector in the vicinity of the proximal end, at least one coaxial cable in electrical communication between the Piezo-electric element and an electrical connector in the vicinity of the proximal end and, electrical communication between the electrode and an electrical connector in the vicinity of the distal end;
b. connecting the ablation device to a source of ultrasonic ablation energy, and a source of ionic liquid;
c. advancing the distal end of the ablation device proximate to the perivascular ablation target;
d. pressing the lateral fenestration against the wall of the blood vessel oriented towards the perivascular ablation target; then,
e. delivering an ionic liquid to the hollow cylindrical structure in a substantially continuous manner; then,
f. activating the Piezo-electric element at a level, frequency, and duration sufficient for ultrasonic ablation of the target perivascular tissue;
whereby, the ionic liquid substantially displaces blood from the space between the vascular wall and the Piezo-electric element, while ultrasonic energy is directed to the vascular wall through the fenestration.

65. A method for ablating perivascular tissue of a patient comprising:
a. inserting an ablation device into a blood vessel of the patient, said ablation device comprising an elongated structure with a distal end and a proximal end, a hollow cylindrical structure located in the vicinity of the distal end comprising at least one lateral fenestration, at least one Piezo-electric element mounted within the hollow cylindrical structure configured for directed ultrasonic emission through the at least one lateral fenestration, at least one channel in fluidic communication between the interior of the hollow cylindrical structure and a fluid connector in the vicinity of the proximal end, at least one coaxial cable in electrical communication between the Piezo-electric element and an electrical connector in the vicinity of the proximal;
b. connecting the ablation device to a source of ultrasonic ablation energy, and a source of ionic liquid;
c. advancing the distal end of the ablation device proximate to the perivascular ablation target;
d. administering an ultrasonic contrast agent into a peripheral vein of the patient;
e. pressing the lateral fenestration against the wall of the blood vessel oriented towards the perivascular ablation target; then,
f. delivering an ionic liquid to the hollow cylindrical structure in a substantially continuous manner; then,
g. applying ultrasonic energy from the Piezo-electric at a frequency and mechanical index sufficient to stimulate contrast enhanced harmonic emissions in the target perivascular tissue, and measuring the level and frequency distributions of the harmonic emissions using the Piezo-electric element; then
h. activating the Piezo-electric element at a level, frequency, and duration sufficient for ultrasonic ablation of the target perivascular tissue; then
i. applying ultrasonic energy from the Piezo-electric at a frequency and mechanical index sufficient to stimulate contrast enhanced harmonic emissions in the target perivascular tissue, and measuring the level and frequency distributions of the harmonic emissions using the Piezo-electric element; then
j. determining the effectiveness of the ultrasonic ablation by comparing the measured harmonic emissions prior to the ablation to the harmonic emissions following the ablation;
whereby, the ionic liquid substantially displaces blood and ultrasonic contrast agent from the space between the vascular wall and the Piezo-electric element, while ultrasonic energy is directed to the vascular wall surface through the fenestration.

66. A method for ablating perivascular tissue of a patient comprising:
a. inserting an ablation device into a blood vessel of the patient, said ablation device comprising an elongated structure with a distal end and a proximal end, a hollow cylindrical structure located in the vicinity of the distal end comprising at least one lateral fenestration, an electrically non-conductive outer surface, at least one Piezo-electric element mounted within the hollow cylindrical structure configured for directed ultrasonic emission through the at least one lateral fenestration, at least one electrode within the hollow structure, at least one channel in fluidic communication between the interior of the hollow cylindrical structure and a fluid connector in the vicinity of the proximal end, at least one coaxial cable in electrical communication between the Piezo-electric element and an electrical connector in the vicinity of the proximal end and, and electrical communication between the electrode and an electrical connector in the vicinity of the proximal end;
b. connecting the ablation device to a source of ultrasonic ablation energy, a source of ionic liquid, and a source of RF energy;
c. advancing the distal end of the ablation device proximate to the perivascular ablation target;
d. pressing the lateral fenestration against the wall of the blood vessel oriented towards the perivascular ablation target; then,
e. delivering an ionic liquid to the hollow cylindrical structure in a substantially continuous manner; then,
f. activating the Piezo-electric element at a level, frequency, and duration sufficient for ultrasonic ablation of the target perivascular tissue; and,
g. applying RF energy to the electrode;
whereby, the ionic liquid substantially displaces blood from the space between the vascular wall and the Piezo-electric element, and the electrode, while ultrasonic energy is directed to the vascular wall through the fenestration, and RF energy is conducted between the electrode and the vascular wall surface defined by the fenestration.

67. A method for ablating perivascular tissue of a patient comprising:
a. inserting an ablation device into a blood vessel of the patient, said ablation device comprising an elongated structure with a distal end and a proximal end, a hollow cylindrical structure located in the vicinity of the distal end comprising at least one lateral fenestration, an electrically non-conductive outer surface, at least one Piezo-electric element mounted within the hollow cylindrical structure configured for directed ultrasonic emission through the at least one lateral fenestration, at least one electrode within the hollow structure, at least one channel in fluidic communication between the interior of the hollow cylindrical structure and a fluid connector in the vicinity of the proximal end, at least one coaxial cable in electrical communication between the Piezo-electric element and an electrical connector in the vicinity of the proximal end, and electrical communication between the electrode and an electrical connector in the vicinity of the proximal end;
b. connecting the ablation device to a source of ultrasonic ablation energy, and a source of ionic liquid;
c. advancing the distal end of the ablation device proximate to the perivascular ablation target;
d. administering an ultrasonic contrast agent into a peripheral vein of the patient;
e. pressing the lateral fenestration against the wall of the blood vessel oriented towards the perivascular ablation target; then,
f. delivering an ionic liquid to the hollow cylindrical structure in a substantially continuous manner; then,
g. applying of ultrasonic energy from the Piezo-electric at a frequency and mechanical index sufficient to stimulate contrast enhanced harmonic emissions in the target perivascular tissue, and measuring the level and frequency distributions of the harmonic emissions using the Piezo-electric element; then
h. applying RF energy to the electrode at a level and duration sufficient for RF ablation of the target perivascular tissue; then
i. applying ultrasonic energy from the Piezo-electric at a frequency and mechanical index sufficient to stimulate contrast enhanced harmonic emissions in the target perivascular tissue, and measuring the level and frequency distributions of the harmonic emissions using the Piezo-electric element; then
j. determining the effectiveness of the RF ablation by comparing the measured harmonic emissions prior to the ablation to the harmonic emissions following the ablation;
whereby, the ionic liquid substantially displaces blood and ultrasonic contrast agent from the space between the vascular wall and the Piezo-electric element and the electrode.

68. A method for ablation of carotid body function comprising:
a. Inserting a vascular access sheath into a superficial temporal artery in the retrograde direction;
b. inserting an ablation catheter though the sheath, the ablation catheter comprising an elongated structure with a distal end and a proximal, a thermal ablation element mounted in the vicinity of the distal end, a mechanism configured for positioning the ablation element against the wall of an external carotid artery in the direction and level of a target carotid body, and a mechanism for connecting the thermal ablation element to a thermal ablation energy source;
c. positioning the thermal ablation element against the wall of an external carotid artery adjacent to the target carotid body; then
d. activating the thermal ablation element at a level and for a duration sufficient to ablate the function of said carotid body.

69. An apparatus for ablation of carotid body function comprising:
a. a catheter comprising a flexible structure with a distal end, and a proximal end, with an RF electrode mounted in the vicinity of the distal end, with a means for connecting said electrode to a first pole of an RF generator in the vicinity of said proximal end, and a mechanical means for pressing said electrode against the wall of an external carotid artery associated with the target carotid body;
b. a perforated structure connectable to a source of pressurized ionic liquid configured for eluding said ionic liquid into an internal carotid artery associated with the target carotid body;
c. a metallic structure associated with said perforated structure connectable to the second pole of said RF generator.

70. The apparatus of claim 69 wherein the means for pressing the electrode against the wall of an external carotid artery comprises a pull wire anchored in the vicinity of the distal end, connected to a user actuator located in the vicinity of the proximal end.

71. The apparatus of claim 69 wherein the perforated structure is a balloon.

72. The apparatus of claim 69 wherein the perforated structure comprises a membranous bladder.

73. The apparatus of claim 69 wherein the perforated structure is a guidewire.

74. The apparatus of claim 69 wherein the perforated structure is a lateral fluid port.

75. The apparatus of any claims 69 to 73 wherein the metallic structure is housed within said perforated structure.

76. The apparatus of claim 69 or 74, wherein said metallic structure comprises perforations.

77. A method for ablation of carotid body function comprising:
a. inserting a catheter into an external carotid artery, with said catheter comprising a flexible structure with a distal end, and a proximal end, with an RF electrode mounted in the vicinity of the distal end connected to a first pole of an RF generator;
b. pressing said electrode against the wall of an external carotid artery associated with the target carotid body;
c. eluding a stream of ionic liquid into the internal carotid artery associated with the target carotid body with said ionic stream in electrical communication with a second electrode connected to the second pole of said RF generator;
d. then passing radiofrequency current between the first electrode and the second electrode through through ionic stream.

78. An assembly for ablation of carotid body function in a patient comprising:
a. An ablation catheter comprising a thermal ablation element mounted in the vicinity of the distal end, comprising, a catheter shaft with a caliber between approximately 3 French and 6 French, with a working length between approximately 15 cm and 25 cm, with the ablation element comprising: a hollow cylindrical structure comprising an electrically insulated outer surface, an inner surface that is at least in part electrically conductive, and at least one lateral fenestration, with at least one fluid channel in fluidic communication between the interior of the hollow cylindrical structure and a fluid connector in the vicinity of the proximal end; a mechanism configured for positioning the thermal ablation element against the wall of a carotid artery adjacent to a carotid body, a mechanism for providing the user with a substantially unambiguous fluoroscopic indication of the position of the thermal ablation element within an external carotid artery, and a means for connecting the thermal ablation element to a source of thermal ablation energy mounted in the vicinity of the proximal end;
b. an arterial access sheath configured for superficial temporal artery access comprising a hollow thin walled tubular structure sized to accommodate a 3 French to 6 French ablation catheter internally, with a working length between 10 cm and 20 cm, a radiopaque marker in the vicinity of the distal end of the tubular structure, and a valve and a fluid port mounted in the vicinity of the proximal end;
c. instructions for use comprising instructions for accessing a superficial temporal artery in a retrograde manner, and positioning the ablation catheter for ablation of carotid body function.
